(19) United States
(12) Patent Application Publication
(10) Pub. No.: US 2026/0284253 A1
(43) Pub. Date: Sep. 24, 2026

(54) FLOWABLE SEALANT COMPOSITION BASED ON RAPID GELLING HYDROGEL PRECURSORS AND PARTICULATE HYDROGELS; PROCEDURES BASED ON THE SEALANT COMPOSITIONS

(71) Applicant: Pramand LLC, Bedford, MA (US)

(72) Inventors: Nelson Bello, Lowell, MA (US); Michael Bassett, Hampton, NH (US); David Giusti, North Reading, MA (US); Caitlin Young, Needham, MA (US)

(21) Appl. No.: 19/572,597

(22) Filed: Mar. 19, 2026

Related U.S. Application Data
(60) Provisional application No. 63/775,783, filed on Mar. 21, 2025.

(51) Int. Cl. A61L 24/00; A61L 24/04; A61L 24/08; A61L 24/10

(52) U.S. Cl. CPC A61L 24/0036; A61L 24/0031; A61L 24/046; A61L 24/08; A61L 24/102; A61L 24/104

(57) ABSTRACT

Compositions, methods, and applications for flowable, particulate-containing compositions with rapid gelling hydrogel precursors are described. The compositions are effective and convenient for forming hydrogels at target tissue locations, with the hydrogels facilitating hemostasis of tissue defects, tissue sealing, and/or space filling, for example. Compositions having entrapped gas can enable high accuracy of delivery to target tissue locations. The compositions can be applied using various delivery vehicles, including syringes and cannulas. Methods for forming the compositions and delivery of the compositions are described. The compositions can be useful for treating fistulas.

### CROSS REFERENCE TO RELATED APPLICATIONS

This application claims priority to copending U.S. provisional patent application 63/775,783 to Bello et al., entitled “Flowable Sealant Composition Based on Rapid Gelling Hydrogel precursors and Particulate Hydrogels; Procedures Based on the Sealant Compositions,” incorporated herein by reference.

### FIELD OF THE INVENTION

The invention relates to sealing compositions comprising hydrogel precursors blended in aqueous solution with swellable hydrogel particulates, in which the sealing composition provides a reasonable delivery time but gels quickly upon contact with bodily fluids. The invention further relates to medical procedures effectively using the sealing compositions, which can provide hemostatic properties in the context of significant bleeds or can provide desirable longer term space filling functions.

### BACKGROUND OF THE INVENTION

Hydrogels have found a range of uses in medical applications for surgical sealing, drug delivery, tissue fillers, spacers, and the like. In the context of wound healing, hydrogel materials can provide a hydrophilic environment to isolate tissue and facilitate healing. For hemostatic and other wound healing applications, existing products have limitations that provide constraints on their effective use. Hemostasis that depends on blood clotting requires the presence of biological factors and can be impaired by anti-clotting drugs or when a patient has a coagulopathy either due to a disease such as hemophilia or due to hemodilution as may occur with traumatic blood loss. There is a need for hemostatic sealants that do not depend on the patient's blood coagulation to stanch the flow of bleeding. There is also a need for hemostatic materials to be introduced within surgical fields that are difficult to visualize due to their anatomic location not being on the surface, due to there being a deeper wound, or due to limited access afforded by less invasive surgery. Thus, hemostats are useful in such situations, and hemostats that are not dependent on blood clotting can be particularly useful. There are also many situations where the fluid leaking from a traumatic or iatrogenic injury, does not have an inherent ability to coagulate. Such fluids may include air, urine, bile, serum, lymph, among others. There is a need to be able to seal such leaks. Sealants that can be used in open as well as less invasive surgery to stop the leakage of such fluids are desirable. Finally, most surgical sealants known in the art currently are applied topically and many of them are synthetic and do not permit tissue ingrowth and thus can end up being barriers to surgical repairs where tissue ingrowth would be desirable.

### SUMMARY OF THE INVENTION

In a first aspect, the invention pertains to a foamed flowable composition comprising:
1) a first component comprising a) a granular hydrophilic material, b) hydrogel precursors comprising an electrophilic-hydrogel precursor having a plurality of electrophilic functional groups and a nucleophilic-hydrogel precursor having a plurality of protonated amine groups, or c) a mixture of granular hydrophilic material and hydrogel precursors;
2) an aqueous solvent, and
3) and entrapped gas. The hydrogel precursors are dissolved and the granular hydrophilic material is at least partially hydrated. Generally, if the first component comprises hydrogel precursors, the aqueous solvent has a pH of no more than about 6.5.

In a further aspect, the invention relates to a flowable composite composition comprising a mixture of a granular hydrophilic material, hydrogel precursors comprising an electrophilic-hydrogel precursor having a plurality of electrophilic functional groups and a nucleophilic-hydrogel precursor having a plurality of protonated amine groups, and an aqueous solvent at a pH of no more than about 6.5. The hydrogel precursors are dissolved and the granular hydrophilic material is at least partially hydrated.

In an additional aspect, the invention pertains to a method for forming a flowable composite composition useful as a tissue sealant. The method comprises dissolving an electrophilic-hydrogel precursor in an aqueous solvent to form an electrophile hydrogel precursor solution; dissolving a nucleophilic-hydrogel precursor in an aqueous solvent to form a nucleophile hydrogel precursor solution; and blending insoluble hydrophilic granular material into a hydrogel precursor solution. The method further comprises combining the electrophilic precursor solution and the nucleophilic precursor solution if separately formed, prior to or following blending the insoluble hydrophilic granular material to form the flowable composite composition comprising a mixture of the electrophilic hydrogel precursor solution, the nucleophile hydrogel precursor solution and the insoluble hydrophilic granular material.

In a further aspect, the invention pertains to a method for the delivery of a medical seal. The method comprises placing a quantity of a flowable composite composition on or in or near a defect associated with tissue to form a fluid barrier. The flowable composite composition comprises a mixture of a granular hydrophilic material, an electrophilic-hydrogel precursor, a nucleophilic-hydrogel precursor, and an aqueous solvent at a pH of no more than about 6.5, wherein the hydrogel precursors are dissolved.

In a further aspect, the invention pertains to a flowable composite composition comprising a dispersion of a granular hydrophilic material in an aqueous solution comprising an aqueous solvent, a dissolved electrophilic-hydrogel precursor and a dissolved nucleophilic-hydrogel precursor. The aqueous solvent is at a pH of no more than about 6.5 and the granules are not fully hydrated.

In a further aspect, the invention pertains to a method of treating a fistula. The method comprises injecting a flowable composite composition into an opening into a fistula. The flowable composite composition comprises a dispersion of a hydrophilic granular material in an aqueous solution comprising an aqueous solvent, a dissolved electrophilic-hydrogel precursor and a dissolved nucleophilic-hydrogel precursor, wherein the aqueous solvent is at a pH of no more than about 6.5.

In another aspect, the invention pertains to a method for forming a foamed hydrogel composite, the method comprising the step of directing a flowable composite composition through an atomizer, wherein the flowable composite composition comprises a dispersion of a hydrophilic granular material in an aqueous solution comprising an aqueous solvent, entrapped gas, a dissolved electrophilic-hydrogel precursor and a dissolved nucleophilic-hydrogel precursor.

### BRIEF DESCRIPTION OF THE DRAWINGS

FIG. 1 is an illustration of the flowable composite composition.

FIG. 2 is an illustration of the flowable composite composition of FIG. 1 after gelation.

FIG. 3 is an illustration of the components of the flowable composite composition of FIG. 1.

FIG. 4 illustrates a method of making the flowable composite composition of FIG. 1.

FIG. 5A is an illustration of a dispenser system suitable for delivering the flowable composite composition of FIG. 1.

FIGS. 5B and 5C are illustrations of dispensers for delivering the flowable composite composition through an atomizer as a spray.

FIG. 6 illustrates a kit for making and delivering the flowable composite composition of FIG. 1 to a tissue.

FIG. 7 illustrates a laparoscopic method of delivering the flowable composite composition of FIG. 1 to a tissue.

FIG. 8A illustrates an open surgical method of delivering the flowable composite composition of FIG. 1 to separate tissue planes exposed during an abdominoplasty procedure.

FIG. 8B illustrates an open surgical method of delivering the foamed flowable composite composition of FIG. 1 to separate tissue planes exposed during an abdominoplasty procedure.

FIG. 9A illustrates a subcutaneous method of delivering the flowable composite composition of FIG. 1 to separate tissue planes.

FIG. 9B illustrates a method of treating an anorectal fistula by delivering a flowable composite composition loaded with antibiotic into a fistula after a cleaning procedure.

FIG. 10A is a photograph of an incision defect on the edge of a liver.

FIG. 10B is a photograph of the laparoscopic delivery of flowable sealant composition onto the defect shown in FIG. 10A.

FIG. 10C is a photograph showing achievement of cohesive sealing and partial hemostasis after the placement of the flowable sealant composition for treatment of the edge incision defect shown in FIG. 10A.

FIG. 11A is a photograph of the delivery of flowable sealant composition onto an abrasion defect.

FIG. 11B is a photograph of the further delivery of flowable sealant composition onto the defect shown in FIG. 11A.

FIG. 11C is a photograph showing achievement of hemostasis after the placement of the flowable sealant composition into the surface defect shown in FIG. 11A.

FIG. 12 is a photograph of the delivery of flowable sealant composition onto an abrasion defect made on a 45° angle surface.

FIG. 13A is a photograph of a crater defect in a liver made with an 8 mm biopsy punch.

FIG. 13B is a photograph of the tip of a 5 ml syringe filled with flowable sealant composition being introduced into the crater defect shown in FIG. 13A.

FIG. 13C is a photograph of the syringe shown in FIG. 13B being further pushed into the crater defect.

FIG. 13D is a photograph showing achievement of hemostasis after the placement of the flowable sealant composition into the crater defect shown in FIG. 13A.

FIG. 14A is a photograph of a 5.0 wt % gelatin formulation with no added air after a 60 second flowability test.

FIG. 14B is a photograph of a 4.0 wt % gelatin formulation with no added air after a 60 second flowability test.

FIG. 14C is a photograph of a 3.5 wt % gelatin formulation with no added air after a 60 second flowability test.

FIG. 14D is a photograph of a 2.5 wt % gelatin formulation with no added air after a 60 second flowability test.

FIG. 15 is a photograph, taken during a flowability test, of a 3.5 wt % gelatin formulation with 72% added air.

### DETAILED DESCRIPTION OF THE INVENTION

A flowable composite, potentially sealing, composition comprises particulates of a hydrophilic polymer within an aqueous solution comprising hydrogel precursors that gel upon contact with bodily fluids. The composite composition may comprise degradable polymers that are resorbed after a suitable period of time, although in some embodiments the compositions are designed for long term or effective permanent placement, such as for bulking. The particulates or granular material generally are hydrogels upon hydration and can be highly absorbent such that swollen particles hold a significant amount of fluid to form a stabilizing mass that is sufficiently cohesive while providing a matrix for the precursors to crosslink around and possibly through. The hydrogel precursors generally are dissolved in the aqueous solution, which can be at a pH that stabilizes the precursors with respect to gelling/crosslinking until contact with bodily fluids raises the pH. The blend of hydrophilic absorbent polymer particulates and dissolved hydrogel precursor compositions can be formulated to be sufficiently stable to provide a suitable pot life while rapidly gelling to provide sealing or other tissue support and may provide hemostasis in response to a significant bleed. The presence of the hydrophilic polymer in the form of granular material serves to stabilize the composition during the gelling time to reduce or eliminate runoff. With appropriate processing, the blend of the granular hydrophilic materials and the hydrogel precursor entraps air and forms a foam, and the foam enhances cohesion of the material such that placement and stability of placed flowable material is enhanced. Foaming can be further stabilized and/or enhanced through the use of an atomizer structure attached to an application, such as a syringe, for the delivery of the flowable composite to the treatment site. A selected amount of added entrapped air can be added. Thus, the composite of the granular material and the in situ formed hydrogels provide functionality that is not provided by the separate components to enable treatment of a range of defects, including obtaining hemostatic results for significant bleeding events. The composite hydrogel material can function for treatment of fistula or the like or for facilitating sealing of tissues separated in a medical procedure. The injectability and rapid gelation provide versatile delivery and suitable deployment within a patient as well as superficial placement for wounds through the skin or other defects. The flowable composite composition cures upon delivery to a target site in contact with bodily fluids to form a crosslinked hydrogel composition comprising a composite hydrogel material. The sealing composition may be particularly useful for certain procedures, such as sealing large abdominal incisions at the end of the procedure. Thus, the composition is complementary to other sealant and hemostatic products.

Improvements in patient care while simultaneously advancing efficiencies for physicians can be built on the foundation of advances in medical products available for treatments using appropriate procedures. Various medical or surgical procedures involve interventions involving stabilization of tissues following the procedure. Such stabilization often involves attenuating or stopping blood loss and may involve mechanical support as well as reduction of infection risk. Hydrogels have found various uses providing assistance with medical procedures due to general compatibility with tissue and the availability of resorbable materials that can dissipate after a suitable period of time. As described herein, the current materials can be delivered internally to a patient, and due to the injectable nature can be suitable for open or low invasive procedures. In additional or alternative embodiments, the flowable composite composition described herein can be applied dermally or transdermally to address defects, such as wounds, lacerations associated with incisions, fistula or the like. Defects refer to wounds or other unnatural conditions suggesting medical intervention or treatment. For example, in order to repair or prevent the formation of seromas and hematomas after surgery, it is desirable that the surgically dissected tissue planes adhere and repair acutely as well as chronically. There is a desire for sealant compositions that are suitable for such repairs. There is also a desire for surgical sealants that may be biosynthetic so as to enable the non sheet or barrier like sealing, for example with a material that contains at least one tissue ingrowth permissive component such as a protein or glycosaminoglycan that can be digested by cellular enzymes to enable ingrowth. Such proteins could include gelatin, collagen and glycosaminoglycans could include hyaluronic acid, chondroitin sulfate, or the like. Upon the sealing of dissected tissue planes, the edges of the connected tissue planes can be further stabilized with suture, staples, bandages, combinations thereof or the like to help maintain secured edges while the hydrogel sealant stabilizes the interior of the healing tissue surfaces. Patients are generally humans, although the compositions and methods generally can be used on pet or farm animals.

Various procedures and injuries suggest the applicability of a sealant to mitigate blood loss and to protect delicate tissue from infection and injury. Various products are on the market to serve this function. Depending on the particular medical indication, specific properties may be desirable. In particular, for surgical procedures, the use of a sealant that is resorbed into benign compounds that are removed by natural processes can be desirable so that no components remain in the patient long term without a need for intervention for their removal. At the same time, the sealant should be biocompatible to avoid excessive inflammation and effective to seal in a relatively short time and without excessive effort to apply the sealant to the wound. Biocompatibility is an accepted concept in the art and may depend somewhat on the application, since some tissues may be more sensitive than others. with appropriate regulatory agencies arbitrating such a characterization. In some embodiments, the sealants generally should provide hemostasis within several minutes, and in some embodiments, hemostasis can be achieved in the context of a significant amount of bleeding. The injectable sealants described herein can provide good hemostatic function for significant bleeds in an easy to apply format with rapid gelation. As described further below, based on the nature of the flowable composites, hemostasis or other appropriate sealing may be achieved without necessarily crosslinking the entire volume of flowable composite, depending on the particular application configuration. Depending on the configuration, it may or may not be desirable or useful to apply pressure, and foaming can stabilize the material without pressure in the appropriate configuration.

The utility of hydrogels has been recognized for some time in the context of biological sealants. Relatively early descriptions of such hydrogels are found in U.S. Pat. No. 6,179,862 to Sawhney, entitled “Method and Apparatus for In Situ Formation of Hydrogel,” and U.S. Pat. No. 8,535,705 to Pathak et al., entitled “Biocompatible Polymers and Hydrogels and Methods of use,” both of which is incorporated herein by reference. Further development of these hydrogels is described in U.S. Pat. No. 8,512,749 to Sawhney et al., entitled “Dehydrated Hydrogel Precursor-Based, Tissue Adherent Compositions and Methods of Use,” incorporated herein by reference. Specific application of in situ gelling hydrogels a spinal sealants is described in published U.S. patent application 2017/0216487 to Sawhney et al., entitled “Low Swelling Biocompatible Hydrogels,” incorporated herein by reference. Corresponding commercial products related to this technology include CraniSeal™ Dural Sealant (Pramand, LLC), Spineseal™ Dural Sealant (Pramand, LLC), DuraSeal® (Integra Lifesciences) and DuraSeal® Xact (Integra Lifesciences).

Hydrogels have also been developed for use as blood vessel sealants for use after medical procedures accessing blood vessels. These hydrogels are superabsorbent such that the hydrogels have a high degree of swelling. See U.S. Pat. No. 8,795,709 to Sawhney et al., entitled Superabsorbent, Freeze Dried Hydrogels for Medical Applications,” incorporated herein by reference. Superabsorbent hydrogels are found commercially in the MYNX™ Vascular Sealant (Cordis).

While the above sealing hydrogels can be very effective for their intended applications, these applications are generally in the context of specific medical procedures with fairly well-defined corresponding wounds. Complementary to these products, hydrogel based medical patches can also be used to provide hemostasis for various wounds or other bleeding sites. Applicant has developed advanced hydrogel based medical patches, suitable for providing hemostasis. This patch has a highly absorbent hydrogel substrate and a layer of in situ gelling hydrogel precursors. The patch provides rapid absorption of fluids due to the substrate, and the drawing of fluids into the substrate helps to drive the rapid gelling of the hydrogel precursors that then provides good adherence to wounds even with high blood flow. Hemostasis can be achieved quickly with high bleeding wounds of various shapes. For example, surgery on organs can be secured with a hemostatic hydrogel patch. The hemostatic patches can be applied internally or superficially/dermal locations as long as the wound has sufficient access for patch placement and maintenance of some pressure. These advanced hydrogel based medical patches are described further in published PCT application WO2023/215453 to Bassett et al. (hereinafter the '453 application), entitled “Water Activated Hydrogel-Based Medical Patches, Flexible Substrates and Methods of Making and Using Such Patches,” incorporated herein by reference.

With respect to other medical uses of hydrogels, hydrogels can be effective drug delivery vehicles or for providing space filling functions. With respect to drug delivery, hydrogels have been found to be particularly beneficial in the context of eye diseases. For example, see U.S. Pat. No. 8,409,606 to Sawhney et al., entitled “Drug Delivery Through Hydrogel Plugs,” and U.S. Pat. No. 9,775,906 to Sawhney et al., entitled “Hydrogel Polymeric Compositions and Methods,” and published U.S. patent application 2018/0085307 to Sawhney et al., entitled “Intracameral Drug Delivery Depots,” all of which are incorporated herein by reference. In a space filling role, hydrogels can provide a protective environment for organs, such as during irradiation, see U.S. Pat. No. 7,744,913 to Noyes, entitled “Fillers and Methods for Displacing Tissues to Improve Radiological Outcomes,” or adhesion prevention, see published U.S. patent application 2022/0143276 to Bassett et al., entitled “Hydrogels Formed In Situ and Composition Design for Intrauterine Use,” both of which are incorporated herein by reference.

The present compositions harness the compelling advantages of in situ crosslinking hydrogels in combination with the dynamic and mechanical stabilization provided by preformed absorbent hydrogels. In an aqueous solution, the hydrogel precursors are dissolved. With avoidance of potential complications from dilution effects that can interfere with gelling, the precursors should crosslink rapidly. But, the precursors are mixed, so they should not crosslink prematurely since then the delivery tool may clog and/or the resulting mass may not stably adhere to the wound to form a good seal and provide hemostasis. The pH can be used to gate the crosslinking so that premature crosslinking does not occur, but the precursor still gels quickly upon contact with bodily fluids, for example, blood or moist tissue. The presence of the granular material (preformed) hydrogel particles, helps to retain liquid at the hemostatic seal and to control dilution of the precursors so that they have sufficient contact to initiate crosslinking through a raising of the pH without excessive dilution that could prevent good gel formation. The flowable sealant can be effective for deep wounds and irregular surfaces due to the ability to conformally fill the wound, which are sites that may be challenging for a patch to achieve hemostasis due to complications of adhering to an irregular surface.

Also, it has been found that with proper processing, the composite flowable material can be deposited as a foam with sufficient entrapped air to effectively form a two component material that has some mechanical integrity for self-supporting shapes. In general, foam formation involves application of mechanical force to blend ambient gas into the material along with appropriate surface tension to provide entrapment of the gas in the material and maintenance of the entrapped gas against dissipation. While the components may introduce a small amount of entrapped air naturally based on the materials used and general processing, a selected volume of added air can be introduced for incorporation into a foam of the precursor composite. In addition to the supply of a gas, such as air, formation of the foam involves some mechanical blending to drive the foam formation. Without the mechanical separation, the gas remains a separate phase, and a foam is not formed. While gas, such as air, can be supplied with the components of the precursor composite, in some embodiments, it can be desirable to provide the components without a large amount of air or other gas such that a controlled amount of gas, such as air, can be supplied to form a consistent foam. The amount of gas supplied with the components of the precursor composite can be estimated from the compressibility. To further support the foaming process and maintaining the foam structure during and following delivery to a treatment site, the composite flowable material can be delivered from the dispenser through an atomizer, such as a luer-connected atomizer on a syringe. The resulting precursor composition at the treatment site has a consistent foam texture, which then gels quickly. The foamed material can be particularly stable and conformable to allow for good sealing of a wound.

The '453 application noted above discusses the option of fragmenting a medical patch to form a filler material. The objective for the formation of the shredded patches is to be able to place the hemostatic material where it is difficult to place the patch. This approach of starting with the patch has several significant drawbacks relative to the present invention. For example, while the precursors are soluble, dissolving the precursors from larger particles can take more time than from a fine powder, so having the sample properly prepared for injection in a uniform and predictable way can be challenging. The designs of the present compositions can be directed to achievement of an appropriate pot life and injectability through a desired needle/cannula dimension, as well as having a selected ratio of components to provide desired properties of the composition. In particular, the present materials allow for foam generation and maintenance over the course of gelation of the precursors.

As noted above, the achievement of the present injectable composite composition involves the formation of a solution/dispersion that is stable for a reasonable amount of time to allow for delivery without clogging the injection tools. This time is referred to herein as the pot life or storage stability. This objective has been a challenge in the in situ formed hydrogel art. Applicant has made significant progress in this regard through the development of a single solution precursor based on electrophile-nucleophile crosslinking with a reasonable pot life and relatively fast gelation. See published U.S. patent application 2023/0338286 to Bassett et al. (hereinafter the '286 application), entitled “Single Solution Hydrogels With Covalent Bonding Formed In Situ, Composition Design and Medical Procedures Using the Hydrogels,” incorporated herein by reference. The design of these precursor solutions generally goes against conventional wisdom where a buffer is thought significant for control of the precursor behavior. Through formulation of the precursor solution with a tolerable amount of added buffer or no added buffer, the gel times can be kept desirably low by allowing more rapid equilibration with the patient's physiological pH, generally about 7.1 and 7.6. The low pH of the precursor solution prior to delivery provides for inhibition of significant crosslinking allowing for a significant pot life, which is essentially the time from preparation of the precursor solution until the viscosity becomes high enough to induce clogging of a delivery catheter.

While the compositions of the '286 application are an important advance in the art, they may not be suitable in this format for more challenging sealant applications. The Examples explore these issues. The data suggests that this is more than a viscosity issue, but it is correlated with the cohesiveness upon delivery. While viscosity and cohesiveness can have some correlation, the delivered material should have moderate viscosity and good cohesion to form a particularly good seal while remaining at the position of application and having facile delivery. Similarly, the two component hydrogel precursors used for sealants, such as CraniSeal™ described above, similarly may not be suitable for challenging bleeding environments and have an added challenge of needing to complete delivery in a relatively short period of time once precursors are mixed in the delivery tool. The compositions herein overcome these challenges to provide an injectable precursor blend with a reasonable pot life and suitable for use in challenging sealing environments.

The flowable composite compositions for the injectable hydrogel sealants comprise two polymer components in an aqueous solution; a granular component and a dissolved precursor component that crosslinks to form a hydrogel following administration to the delivery site. As described further below, the aqueous solution can be formed with sterile saline or similar suitable aqueous liquid at a selected pH. The granular component generally is an absorbent particulate hydrogel, or xerogel prior to hydration, that can hold a significant amount of water. In exemplified embodiments, the granular material is formed by pulverizing a gelatin foam material, or other similar insoluble hydrophilic polymer material, that can absorb a significant amount of water, generally at least about 3 times the polymer weight. The material can be provided as friable chinks for forming the granular material. While the pulverizing process breaks up the cell structure of the initial foam material, the resulting granular material still absorbs significant amounts of aqueous fluids, which may contribute to liquid-foam formation for the flowable composite. The combination of the hydrogel precursors and the hydrophilic hydrogel granular component combine to reduce surface tension appropriately to form the foam upon exertion of mechanical work to blend in air bubbles. Some air can be introduced naturally from the processing, and an additional amount of air can be added with a selected volume to achieve the desired foam properties. It is surprising found that delivery of the flowable composite compositions through an atomizer provides for stabilization of and potentially further stimulates delivery of a well-characterize foamed deposit of the flowable composite compositions. The foamed material, flowable composite composition can have the properties of a semi-solid material, which can be effectively described as a non-Newtonian fluid that does not flow significantly as deposited, but can be shaped or separated to contour the as-deposited foam. In contrast with foamed embodiments, non-foamed embodiments may involve application of pressure or other surface stabilization with a wet sheet material, which can result in an undesirable adhesion or otherwise risk disruption of the composite hydrogel material that may be being used to establish hemostasis.

As described below, the granular materials are generally hydrated in preparation for delivery of the composite, so the composite precursor may not be significantly absorbent. Nevertheless, the hydrated granular component can provide significant cohesion that facilitates placement and maintenance of the composite precursor while the hydrogel precursors crosslink and form the composite hydrogel material. Foamed thermally crosslinked gelatin with a fractured cell structure is a desirable starting material for forming the granular component, and further suitable materials are described below. To avoid any confusion, the foamed thermally crosslinked gelatin is a solid material that can be hydrated into a hydrogel, while the foamed flowable composite composition can be dissolved prior to covalent crosslinking of the hydrogel precursors in the presence of the granular materials, such as thermally crosslinked gelatin, disbursed as hydrogel particulates. The hydrogel precursors comprise an electrophilic precursor and a nucleophilic precursor that covalently crosslink with each other under suitable conditions to form a highly crosslinked hydrogel. The crosslinking reactions are well established and are described in detail below. The precursor compounds generally have a hydrophilic core and a plurality of crosslinking functional groups. One or both precursors generally have a macromolecular core structure, which can comprise a highly branched polymer structure, such as a star polymer, a dendrimer or comb structure. The crosslinking reaction can be pH gated to control the initiation of the crosslinking reaction.

In some embodiments, the granules of the composite composition can comprise particulate hydrogel/xerogel, which can be sufficiently hydrophilic to allow for interaction with the dissolved hydrogel precursors into the particulate hydrogel structure to allow crosslinking of the hydrogel precursors to form an intertwined hydrogel network around the granular structures to form a polymer composite. The granular, hydrophilic particles can be irregularly shaped, although spherical, ellipsoidal, or rectangular shapes can perform appropriately in the composite. When formulating the dispersion corresponding to the flowable composite composition for delivery, the granular material, which becomes hydrated, can be well mixed with the precursors prior to delivery. As the gelling rapidly progresses, the intertwined nature of the composite polymer can result in significant stability while maintaining a highly hydrated state with a conforming nature and a gentile surface interfacing with the damaged tissue. In some embodiments, the hydrophilic granules are well hydrated prior to delivery, and the resulting composite can have little or no swelling upon completion of crosslinking and immersion in fluid. In alternative embodiments, the crosslinking in situ formed hydrogel can contribute to a desired degree of swelling. In further alternative embodiments, the hydrophilic granular material can be added to hydrated hydrogel precursor with insufficient time to fully hydrate to equilibrium the hydrophilic granular material. If not fully hydrated, the further hydration of the hydrophilic granular material generally results in swelling, so the degree of swelling may be controlled to some degree by the timing of the addition of the hydrophilic granular material to the injection fluid. While little or no swelling may be desirable for some embodiments, the composite materials can be formed to have a desired degree of swelling. Swelling may be desirable for use in procedures where the delivery site has oozing fluid. The degree of swelling and corresponding lack of full hydration can be controlled by the hydration time and is also influenced by the nature of the hydrophilic granules as well as the hydrogel precursors.

Processing to form a foamed flowable composite involves the introduction of air or potentially other gas in a suitable amount for incorporation into the flowable composite upon introduction of effective mechanical work to form the bubble structure within the composite. Suitable gases include, for example, air, nitrogen, carbon dioxide, ultrasound contrast agent, as described below, other biocompatible gases and mixtures thereof. The exemplified foamed gelatin source combined with the pulverizing process to form granules results in the formation of the flowable composite as a foam, and these parameters can be reproduced and extended, as desired, to account for other appropriate granular sources and other corresponding processes. Some air is introduced by the materials that are combined, and it can be expected that foamed gelatin can introduce some entrapped air. An additional volume of air/gas above this baseline can be introduced at a selected volume. It is found that the amount of entrapped air can be self-limiting, so if it is attempted to introduce additional air above a certain amount, the air above the limit may remain unentrapped. The amount of mechanical work can be readily introduced manually, and the amount of air can be adjusted within reasonable amounts to achieve desired foam properties. The combination of materials in the flowable composite nicely provides the desired surface tension properties to support maintenance of the foam. The flowable composite structure is compatible with rapid gelling from the crosslinking of the precursors upon contact with bodily fluids that raise the pH.

In some embodiments, the crosslinked hydrogel composition can be degradable following delivery so that the material naturally clears from a patient without intervention. The in situ degradation can be due to hydrolysis in an aqueous environment, enzymatic or other biological effects or a combination thereof. In some embodiments, the in situ polymerized hydrogel primarily degrades by hydrolysis and the granular hydrogel component primarily degrades by enzymatic activity, although any appropriate degradation pathway can be appropriate independently for each component, and the pathways may or may not be different for the different components. Thus, the degradation times for the in situ hydrogel and the granular hydrogel components may or may not be roughly the same. The design of the composite composition can take into account the time for degradation of the material. In some embodiments, the crosslinked hydrogel composition degrades in no longer than about 14 days, in further embodiments no longer than about 10 days or in other embodiments less than about 3 days with respect to all components of the composite to avoid encapsulation of the granular material by bodily processes. In other embodiments, it can be desirable for the composite composition, or a portion thereof, to persist for significant periods of time, such as several weeks or longer. In further embodiments, it may be desirable for the composite composition to be essentially permanent, which can effectively be five years or more, under physiological conditions. A person of ordinary skill in the art will recognize that additional ranges of degradation times within the explicit times above are contemplated and are within the present disclosure.

Thus, the injectable, flowable composite composition generally comprises solvent and hydrogel polymeric material, i.e., polymer and polymer precursors. While the polymer precursors are initially soluble, upon crosslinking, these become an insoluble mass. The granular polymer material is initially insoluble and remains insoluble until degrading. For the purposes of allocating concentrations, the “solvent” is considered to comprise water and soluble components other than the hydrogel precursors and optional thickeners or other additives that can physically crosslink or become embedded with the hydrogel components upon solvent removal. Soluble components can comprise salt, generally sodium chloride, as well as any other optional additives that are not maintained in the cured hydrogel. Optional added air or other gas can be introduced for foaming, and the selection of ingredients can introduce a baseline amount of entrapped air. Optional additives generally can comprise a visualization agent, such as a dye, and may comprise other process aids, such as a thickener. Salt can be added to approximate physiological ionic strength, conventional IV ionic strengths or other reasonable values.

The polymeric material of the flowable composite composition comprises a granular hydrogel composition and soluble hydrogel precursors. To be a component of an injectable composition, the granular material should have particle sizes significantly below the needle/cannula inner diameter. If the flowable composite composition is to be passed through an atomizer, the particles may not necessarily be smaller or significantly smaller than the atomizer orifice since the granular particles are generally compressible, and the short transit time combined with the significant pressure differentiation at the atomizer may allow compressible particles to traverse through the atomizer, although the particles should be smaller than a catheter or the like, if present, whether or not there is an atomizer. In some embodiment, the granules can have particle sizes below one millimeter with corresponding smaller average particle sizes, depending on the prescribed injection parameters, and generally significantly below these sizes. Larger granules should be avoided to prevent needle/cannula clogging. The shapes of the granules generally are influenced by its method of production. Irregular particle shapes can influence rheology of the injectable composition as well as the mechanical properties of the gelled material. The granular material may or may not be formed from a porous matrix, such as by milling, mechanical fragmentation, sonication or other suitable approach, and fragmenting a porous matrix can effectively form irregularly shaped granules that are effective at forming an injectable composition with desirable properties. Absorbent substrates can be formed from natural materials, such as collagen, gelatin, cellulose, starch, hyaluronic acid, or the other similar biopolymers, or from synthetic hydrogels, such as polymers comprising poly(ethylene glycol), poly(vinyl alcohol), or other water soluble or water swellable synthetic polymers, or combinations with biopolymers. Exemplified granules are formed from thermally crosslinked foamed gelatin sections that are fragmented.

The hydrogel precursors generally comprise a first hydrogel precursor with a plurality of electrophilic functional groups and a second hydrogel precursor having a plurality of protonated amine groups, generally primary amines. In particular, the nucleophilic amines can be protonated to form a cationic ammonium group, which protects the amine from nucleophilic reaction until it is deprotonated. A halide, such as chloride, or other strong acid conjugate anion can be the counter ion. The electrophilic functional groups and the nucleophilic functional groups can react to form covalent crosslinks once the blend is hydrated and the ammonium group is deprotonated due to contact with alkaline pH bodily fluids, which correspondingly increases the pH. Even with the use of a fine powder form of the precursors, processing should verify the proper dissolving of the precursor compositions for use in the injectable composition.

One or both precursor compositions, electrophile or nucleophile, can be macromers generally having biocompatible and water soluble cores with pendent reactive functional groups for crosslinking. When the core is a polymeric region that is water soluble, polymers that may be used can be natural or synthetic polymers. Suitable polymers for the core can include polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, poloxamers, such as Pluronic® F-127; as well as polyoxazolines, polyvinyl alcohol (“PVA”); poly(vinyl pyrrolidinone) (“PVP”); and polysaccharides, such as hyaluronic acid, chitosan, dextran or digested cellulose and derivatives thereof. Based on proven results in a range of medical products, PEG based precursors are exemplified and provide desirable results.

In some embodiments, the flowable composite composition comprising the hydrogel precursors has no added buffers, and results demonstrate rapid and good gelation without any buffer. Some appropriately selected buffer potentially can be present in low concentrations without disrupting the function of the precursor crosslinking. Carbon dioxide from air or as present in a laparoscopic environment can dissolve into water to form carbonic acid, which can provide low amounts of buffer capacity and a slightly acidic pH. Carbonic acid evaporates as carbon dioxide rather than concentrating as water is removed, and bodily fluids are already equilibrated with atmospheric carbon dioxide. Inappropriately selected buffer can be undesirable either due to causing undesired premature crosslinking of the precursors prior to use or to slowing of the crosslinking after application to a tissue.

Specifically, the exemplified NHS-amine crosslinking reaction does not occur significantly in unbuffered aqueous systems in the presence of the amine acid conjugate since the protonation acts as a protecting element for the amine group. Unreacted co-dissolution may be obtained by forming single solutions with the electrophilic polymer and nucleophilic polymer acid conjugate in unbuffered or weakly acidic buffered solutions that naturally reside at lower pH (generally a pH 2-5). The presence of the granular component does not significantly alter the crosslinking behavior of the hydrogel precursors.

Generally, the hydrogel precursors have amines at a pH prior to delivery of no more than 6 pH units and a buffer concentration within tolerances or no added buffer. Dissolved carbon dioxide provides a low amount of carbonic acid, which can be considered a baseline and not an added buffer. Physiological fluids are naturally buffered to account for the overlapping presence of dissolved carbon dioxide, and the presence of dissolved carbon dioxide does not interfere with the performance of the hydrogels.

An electrophilic precursor component can have a suitable functional group to crosslink with an amine group to form the hydrogel. The crosslinking functional group is generally pendent on a hydrophilic core, which is generally polymeric, to provide a moderate molecular weight. The precursor generally has three or more crosslinking groups to provide for a highly crosslinked hydrogel. The precursor may or may not have degradable functional groups, which may degrade by hydrolysis, enzymatically or through another biodegradation pathway. The nucleophilic precursor generally comprises three or more amine groups, which generally are pendent on a hydrophilic core. The hydrophilic core of the nucleophilic precursor can be polymeric. The amines in the precursors generally substantially deprotonate under physiological conditions to render the amines available for crosslinking. The protonated amines are protected from crosslinking by the acidification of the amine, which stabilizes the mixed precursor solution. The ability to rapidly deprotonate under physiological conditions allows for fast crosslinking without a basic buffer.

The precursor powder can be dissolved in saline or similar sterile aqueous solution to form a precursor solution as a step in forming the injectable composite composition. The granules are generally supplied in a dry form such that they are stable for storage prior to use. The granules can be mixed directly with the precursor solution or combined with the precursor solution following initial hydration with saline. The timing of the hydration of the granules can be adjusted to select desired properties of the flowable composite composition upon delivery. In any case, the precursor solution and the granules can be well mixed prior to injection. Additional mixing can be used, which can optionally further fragment the granules. The granules generally are hydrated for at least about 1 minute. The resulting flowable (injectable) composite composition generally has a pot life of at least 15 minutes and generally at least an hour, where the pot life indicates the amount of time following mixing during which the composition remains injectable due to little or no gelation/crosslinking.

In some embodiments, the flowable (injectable) composite composition has desirable properties for application to a wound for sealing. The composite generally has appropriate cohesion such that it should not readily flow off of the placement site even with some encouragement by gravity. The cohesion and corresponding surface tension helps to resist dislodgement by modest amounts of fluid that may impinge on the sealant after delivery. At the same time, the composition is generally wetting of tissue surfaces and conformal, so that the space of the wound can generally be conformally filled by the composition. Rapid gelling can help to maintain the composition at its delivery location. The viscosity of the composition can be adjusted by the relative polymer content as well as optionally through the addition of a biocompatible thickener, such as hyaluronic acid, soluble gelatin, lecithin, soluble starch, polyethylene glycol, other biocompatible natural or synthetic polymers and mixtures thereof.

Due to the injectability, the sealant provided by the flowable composite composition can be used in a wide range of procedures. For example, the sealant-flowable composite composition can be used for open surgical procedures, minimally invasive procedures, such as laparoscopic procedures, percutaneous wounds, dermal wounds, percutaneous biopsy sites and the like. The flowable sealants are also well suited for traumatic wounds such as gunshot wounds or knife penetrating wounds where the sealant composition can be delivered deep into the wound with a blunt elongated applicator. For some of these procedures, the flowable composite compositions, provided as an injectable sealant, can be used as an alternative to a sealing patch, while for other procedures, an injectable sealant can be used at locations that are difficult to reach or unreachable with a patch. For percutaneous wounds, the space filling nature and the adjustable volume may dictate a desirable use where a patch may be less than ideal, and the mechanical properties may provide desirable outcomes for larger less defined wounds, based on the cohesive nature of the composition, relative to the use of sealants, such as CraniSeal™, that are very effective for well-defined surgical wounds that are also generally subject to stabilization and closure using suture and/or staples. Also, for assisting the sealing of larger surgical incisions, such as associated with abdominal surgery, the composite sealant can effectively seal along the extended closed incision as an effective seal, which may support and potentially simplify suturing or stapling of the closed incision.

The foam nature of the flowable precursor helps to maintain the placement location of the flowable precursor, which can be particularly desirable if the placement location is not horizontal or otherwise subject to potential disruptive forces. In some embodiments, after application, pressure can be applied to facilitate forming a good seal. Pressure can be applied with an appropriate material, such as wet gauze or a more non-stick material, that can be removed after a short period of time allowing for a significant degree of gelation. However, even with relatively non-stick material, removal of a material used to apply pressure can disrupt the sealing wound and result in a rebleed. If desired, after initial gelation, but prior to full crosslinking, excess material can be removed with a spray of fluid or mechanical removal. Foaming of the flowable precursors may provide a deposit that is sufficiently stable to allow for a diminished benefit from the application of pressure, and the use of an atomizer in the deposition can further stabilize the deposited composite precursor composition to further decrease the benefit of applying pressure and allowing for shaping of the deposited material around the treatment site. Due to the hydrated nature of the granular material, the flowable composite is not highly swelling, so bodily fluids are not aggressively drawn into the composite upon contact. Nevertheless, gelling occurs at the contact point sufficiently to gel the composite at the contact locations to provide hemostasis or similar stabilization of the wound adjacent the contact locations, such that careful removal of excess flowable composite that has not gelled does not disrupt the seal formed by the composite. In some cases, pressure can be applied by overlying tissues that are intended to eliminate any potential spaces that could result in the formation of a seroma or a hematoma. In this case the overlying tissue itself becomes the substrate for applying pressure and resultingly adheres to the underlying tissues upon which the sealant composition has been applied for healing in this configuration.

The injectability also provides for the use of the composite hydrogel for filling a fistula or the like. Fistulas are unnatural connections, generally but not necessarily tubular, connecting two body portions that can be cavities. These can be associated with the gastrointestinal tract, such as anal fistula, the urinary tract, pulmonary, or other organ systems. Fistulas can be open on both ends or only one end and are subject to infections, potentially serious, as well as causing pain for the patient. Permacol™ collagen paste has been used for treatment of anal fistula. See, Permacol for treating anal fistula, Medtech Innovation Briefing, National Institute for Health and Care Excellence, Great Britain, 23 May 2017, incorporated herein by reference. Clinical studies on the use of Permacol™ for fistulas have not yielded completely satisfactory results. Studies suggest a healing rate of about 53.5%, and for patients with complex fistula, the success rate was only 20%. Vollebregt et al., “Efficacy of Permacol injection for perianal fistulas in a tertiary referral population: poor outcome in patients with complex fistulas,” Colorectal Disease, 2021, 23; 2119-2126, incorporated herein by reference. In contrast with the paste material, the flowable composite compositions can be delivered in an appropriately flowable state such that precursors can space fill prior to gelling. The flowable composite compositions are amenable to incorporation antibiotics and/or other therapeutic agents. Once crosslinked the product composite material can have a relatively high modulus such that it can remain secure in the fistula as it heals and can have a degradation time selected to provide a high degree of healing success.

Once crosslinking is completed, the crosslinked material generally provides a good seal with relatively robust mechanical properties. These properties can reduce the chances of inadvertent dislodgement and/or loss of sealing function and serve to prevent the formation of potential spaces which can fill with blood or serum that is leaking from the tissues below.

Due to the relatively long pot life, the flowable composite composition can be prepared prior to the start of many procedures. The foam structure is generally also maintained for a reasonable time period. In other embodiments, the flowable composite composition can be prepared at an intermediate stage or near the end of the procedure. As demonstrated in the examples, the flowable composite composition used as a sealant is suitable for providing hemostatic efficacy, but it can be used for sealing of wounds where excessive bleeds are not taking place, where the sealant nevertheless provides a protective environment for healing and a barrier to infection. While being particularly effective for deep wounds, generally, the flowable composite compositions can be used as a sealant for wounds where the defect has a depth of about 0.1 mm to about 5 cm and/or a surface area of from about 0.1 cm² to about 500 cm². While the composite composition can be suitable for placement in difficult to reach locations, such as through a laparoscope, trocar or the like, the cured hydrogel can provide effective properties for covering a range of wounds with varying amounts of sealing function. In further embodiments, the composite composition can be used for space filling applications, and injectability can provide significant flexibility in this regard. The composition can be useful for spatial separation of tissue or for filling voids or deficiencies in tissue for cosmetic and/or reconstructive purposes or for supplementing performance of other medical procedures. Such procedures may include abdominoplasties, face lifts, mastectomies, and several procedures that may involve the dissection of tissue flaps. Example 3 demonstrated the use of the flowable composite composition for the sealing of an abdominal tissue flap. The precursor composite composition can be delivered to form a crosslinked composite material that is effective as a sealant for a defect having a structure that forms a liquid or gas leak, in which a fluid barrier is formed comprising a hemostatic barrier, a liquid barrier, a gas barrier, or a combination thereof. Leaking liquids can be, for example, blood, urine, bile, serum, lymph or other biological fluid. For example, in the treatment of a wound associated with a bowel perforation, the sealant can function both as a liquid barrier and a gas barrier. Such compositions can also be useful for the filling of fistulae so as to eliminate the tract with a sealant that is permissive of tissue ingrowth, so as to seal it acutely and also allow for a robust chronic repair post absorption of the sealant composition.

### Flowable Composite Composition and Composite Hydrogel Material

The flowable composite composition comprises an insoluble granular hydrophilic material, generally a hydrogel that may be porous, soluble hydrogel precursors, an electrophilic precursor and a nucleophilic precursor, in an aqueous solvent. The flowable composite composition generally is formulated to be injectable through a thin cannula/catheter or needle and may be sprayed. The flowable composite composition is a dispersion of the hydrated granular component in a solution of the dissolved precursors. In some embodiments, the flowable composite composition comprises entrapped gas within a foam. Spraying through an atomizer has been found to facilitate maintenance of foam properties that can be introduced into the flowable composite composition. The dispersion generally has a slightly acidic pH that inhibits crosslinking of the precursors, and the dispersion generally has little buffer capacity and little or no added buffer. The effective lack of buffer capacity provides for rapid gelling of the hydrogel precursors upon a raising of the pH resulting from contact with bodily fluids. The flowable composite composition comprises aqueous solvent, dissolved hydrogel precursors, granular hydrophilic particulates and optional additives, as well as in some embodiments entrapped gas, such as air. Upon crosslinking of the hydrogel precursors, the flowable composite composition becomes a crosslinked hydrogel as a component of a composite hydrogel material.

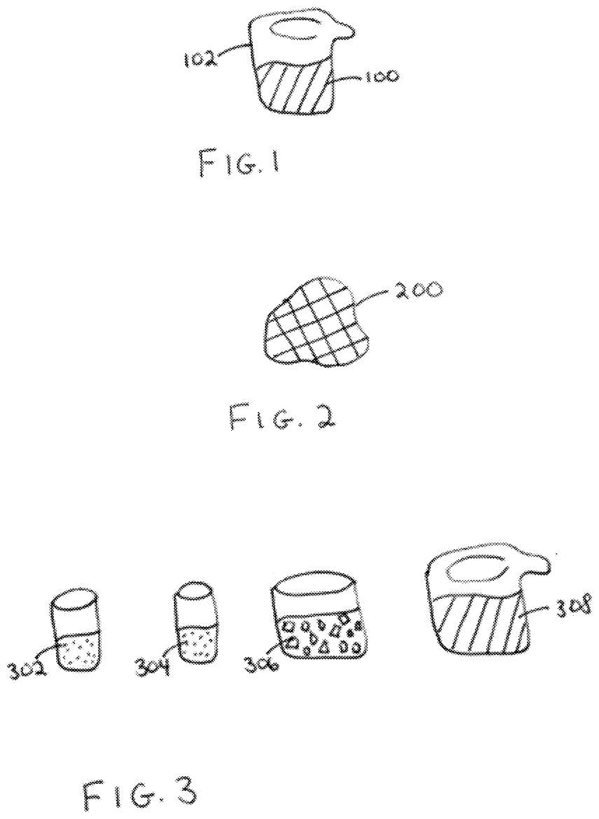

The ability to form well mixed precursors in solution with a reasonable pot life but reasonably rapid gelling provides a valuable platform for delivery of therapeutic biocompatible materials. For relevant embodiments, a foam state of the material does not seem to be significantly detrimental to the crosslinking process. FIG. 1 is an illustration of flowable composite composition 100 in container 102. The illustration highlights the pot life of the blended composition. The use of well dissolved precursors alleviates any concerns regarding the degree of solvation. The design of the precursors and the nature of the solution nevertheless provides for rapid gelation after sufficient contact with bodily fluids allows for an increase in pH. FIG. 2 is an illustration of flowable composite composition 100 after gelation to form patch 200. The particular use of the platform to form a high performance general sealant exploits these desirable feature in an insightful way, although the composites can be useful in other non-sealant applications as noted herein.

The concentrations of the various components influence the delivery properties, the gel time and the properties of the crosslinked material. As used herein and as is common in the polymer arts, crosslinking and curing are used essentially interchangeably, and in the present context, these terms refer materially to the gelling process in which the flowable composition because a stable unitary mass. The concentration can be characterized by the total concentration of solids as well as the proportions of the difference components of the solids. As used herein, the solids include the granules, any non-volatile property modifiers, additives, such as therapeutics, organic visualization agents, and the hydrogel precursors since they become insoluble solids upon crosslinking even though they are dissolved for delivery. The solvents would be distinguishable from the solids, and salts added to the solvent can be considered part of the solvent, for convenience and since these would dissolve and be removed by biological fluids and not become part of the ultimate crosslinked gel.

As noted above, significant properties of the flowable composite composition are the viscosity and the cohesion. In some embodiments, the delivered flowable composite is in a foam state with entrapped gas that can provide some structure without solids form. Hydrogels have been used to form solid foams with a cell structure which can be associated with large liquid absorption. Conceptually, the crosslinking should occur relatively quickly relative to dissipation of bubbles in a liquid foam template for the resulting solid foam. Generally, more stable liquid foams are associated with surfactants, and it is not immediately clear why the combination of the hydrophilic granular material and hydrogel precursors form liquid foams that are stable for at least appropriate times for use in the procedures contemplated for the flowable composites. It is equally unclear why deposition of the flowable composite composition through an atomizer during deposition would facilitate maintenance of the foamed structure of the deposit since atomizers are not generally associated with foaming, although some building materials involve sprayed foam.

Generally, desired properties can be achieved with solid concentrations for the flowable composite composition in ranges from lower limit values of about 2.5 wt %, in some embodiments about 3 weight percent (wt %) solids, in further embodiments of about 4 wt % solids, and in additional embodiments of about 5 wt % solids to, for any of the lower range limits to upper range limit values of about 15 wt % solids, in further embodiments of about 18 wt % solids, in additional embodiment of about 20 wt % solids, in some embodiments of about 25 wt %, and in other embodiments of about 40 wt % solids. With respect to the solid content, the quantity is divided between the granular hydrophilic polymer and the hydrogel precursors as well as optional thickeners, visualization agents, therapeutics, any other additives, and combinations thereof that incorporate into the gel. The relative amounts of these components also influence the pre-gelling and post-gelling properties, as described further below. The polymer solid content can be evaluated based on the weight from the added components. A polymer solid content too low can result in undesirable dilution effects that can slow gelling, and a polymer solid content that is too high can result in slow and potentially nonuniform gelling due to slower deprotonation of the amines. In general, the hydrogel polymer component is divided between the granular and the precursor components and for evaluation of the concentrations with the total of the granular and dissolved precursors being 100% of the hydrogel polymer component, in which granular is considered to include all insoluble dispersed polymer component of any type, including but not limited to the granular hydrophilic polymer component. To the extent that a thickener, such as hyaluronic acid, becomes physically crosslinked with the hydrogel components, it is not considered part of the hydrogel polymer component, but it can be part of the solids. The relative amount of dissolved hydrogel precursors on a weight basis relative to the total hydrogel polymer component can be in a range with a lower limit of about 5% in further embodiments of about 7.5%, in additional embodiments of about 10% and in other embodiments of about 15% to, for any of the lower limit values, an upper limit value of about 35%, in further embodiments of about 40%, in additional embodiments to about 45%, in other embodiments to about 50%, and in some embodiments up to about 60%. The remaining portion of the total hydrogel component besides the hydrogel precursors is the granular component. A person of ordinary skill in the art will recognize that additional ranges of solid concentrations and relative polymer concentrations of the hydrogel precursors or granular polymer component within the explicit values above are contemplated and are within the present disclosure. Concentrations of optional additives is presented below. The polymer precursors can comprise one or more types of electrophilic precursor compounds and one or more types of nucleophilic precursor compounds.

The solvent can be or comprise purified/sterile water (e.g., water for injection), unbuffered saline, or any physiologically acceptable aqueous solution having appropriate (low or no) buffer capacity. Choice of fluid may be influenced by particular application, although sterile saline for intravenous use (physiological saline) is generally readily available to medical practitioners and safe for most or all uses. In particular, intravenous sterile saline generally is about 0.9 percent weight per volume NaCl or equivalently expressed as 9.0 g NaCl per liter. Effects of buffers can be complicated and depend on both the pKa and concentration. Also, many suitable physiological buffers can be polyprotic, which further complicated their effects. Buffer issues are discussed further below, but for most applications, more desirable results can be achieved with no added buffer, and a low or effectively zero buffer capacity can be desirable.

The granular hydrophilic polymer can be characterized by the average particle size, porosity/density, absorbance/particle shape, and composition. The average particle size selection can be influenced by the flowable nature and the cannula/needle size intended for use. Average particle size can be evaluated in a dry state or in a dispersion. Injectability can provide a cutoff for the largest particles since larger particles may clog the needle/canula, although the injection force can further fragment the particles with some corresponding injection resistance. The hydrated granular particles are generally compressible or deformable. Generally, the average particle size is no more than about 3 millimeter, in further embodiments no more than about 2.5 millimeter, in some embodiments in a range from a lower value of about 10 microns to an upper value of about 2 mm, in other embodiments from about 20 microns to about 1 mm, and in additional embodiments from about 25 microns to about 500 microns. Alternatively, the average particle size can be specified from a pragmatic perspective with respect to a type of catheter or orifice that the precursor composite composition can effectively be dispersed through. The particle size can correspond to the largest length connecting two points on the particle surface. A person of ordinary skill in the art will recognize that additional ranges of average particle size within the specific ranges above are contemplated and are within the present discloser.

As noted above, the granular hydrophilic polymer has particle shapes influenced by the composition and formation approach. Examples are obtained using a foam of thermally crosslinked gelatin that is fragmented to form the granular material. The resulting particles have an irregular shape based on the fragmenting of the cells of the gelatin foam. The irregular shapes of the particles can be conducive to increasing the viscosity of the flowable composition as well as to the formation of a cured composite with desirable mechanical properties. Due to the hydrophilic nature of the material, the particle can swell with significant amounts of water even if a pore structure is not maintained in the particles.

Exemplified granular hydrophilic polymer is formed from thermally crosslinked gelatin, and this material is described further below. Other biocompatible hydrophilic polymers, generally hydrogels, can also be useful for the granules. In particular, any reasonable medical hydrogel formed into suitably sized and shaped particle can be effective with an appropriate absorbance of aqueous solution. Suitable materials for the granules can be formed, in principle, from natural polymers, synthetic polymers or a combination thereof. Synthetic hydrogel polymers for the granules include, for example, polyesters, polyurethanes, polyethylene glycols, poly(vinyl alcohol) or other reasonable synthetic polymers. Natural materials are generally processed appropriately for inclusion in medical products, so they are generally modified to varying degrees from their natural form. Nevertheless, natural materials can provide desired properties suitable for the material of the granules, including high absorption of aqueous solutions and degradation in in vivo applications in reasonable period of time. Suitable natural materials include for example, polysaccharides and materials derived from extracellular matrix proteins. For example, polysaccharides that are derivatives of cellulose, pectin, starch, hyaluronic acid or chitosan can be used for making absorbent sheets. Commonly used materials are forms of cellulose that include, for example, ester and ether derivatives, such as cellulose acetate, or ethylcellulose. Oxidized cellulose is a material that aids in hemostasis, but this material is generally believed to be poorly absorbed and may cause postoperative complications, and hydroxycellulose, nitrocellulose or other forms can be appropriate. In some embodiments, the cured composite material can be long lasting or permanent, and suitable compositions can be selected for the granules, such as highly crosslinked gelatin or collagen.

Collagen is an extracellular matrix protein that in native form can be found in triple stranded fibrils. Purified collagens can take various forms, and gelatin is a partially hydrolyzed form of collagen. Collagen can be derivatized in other ways, if desired. Medical grade collagen and gelatin is available commercially, optionally with crosslinking, such as glutaraldehyde crosslinking, and dried, such as lyophilized. While glutaraldehyde crosslinking can stabilize collagen or gelatin, strong glutaraldehyde crosslinking can significantly increase persistence times and can have some toxic side effects. Highly crosslinked collagen and collagenous tissue has a long history of use in long term implanted prostheses, such as heart valves, and these materials could be used for forming granules.

In some embodiments, before forming the granules, the hydrogels can be foamed and dried. The dried material can be formed into granules using manual fragmentation, using a mill, or the like. Foamed medical starch is sold under the trademark, SealFoam™. Particles of medical grade starch are described in U.S. Pat. No. 9,708,416 to Malmsjo et al., entitled “Microspheres of Hydrolyzed Starch With Endogenous, Charged Ligands,” incorporated herein by reference. Degradable crosslinked hyaluronic acid is described in U.S. Pat. No. 10,077,320 to Karlsson et al, entitled “Process for Forming a Crosslinked-Hyaluronic Acid Product,” incorporated herein by reference. Exemplified embodiments use thermally crosslinked foamed gelatin.

Gelatin can be thermally crosslinked to mechanically stabilize the material suitable for the granular component of the composite, while avoiding introducing any toxicity or excessive persistence. Prior to crosslinking, the gelatin can be placed in the desired shape and then heated. If the desired granular sizes are not directly formed, the resulting material can be fragmented, milled or otherwise ground. The length of thermal crosslinking generally influences the persistence times, and these relationships may depend to some degree on whether or not the thermal crosslinking is performed directly on the granules or in a larger format that is subsequently subjected to granule formation. In general, thermal crosslinking can be performed at temperatures from about 100° C. to about 200° C., and in further embodiments from about 120° C. to about 180° C. for time from about 15 minutes to about 24 hours and in further embodiments from about 25 minutes to about 6 hours. A person of ordinary skill in the art can adjust the time and temperature to achieve desired properties, such as mechanical stability along with persistence times after in vivo implantation. Using the thermally crosslinked gelatin material, the initial gelatin material can be a foam, a non-woven tufted material, or a non-woven felted material. A person of ordinary skill in the art will recognize that additional ranges of temperatures and times within the explicit ranges above are contemplated and are within the present disclosure.

In some embodiments, gelatin can be chemically crosslinked as an alternative or in addition to thermal crosslinking. Chemical crosslinking of gelatin is known and can be performed to provide selectable relatively short degradation times or long term stability. Suitable crosslinking agents include, for example, aldehydes, dialdehydes, isocyanates, diisocyanates, carbodiimides and alkyl halides. It is possible to perform gelatin chemical crosslinking to obtain selectable degradation times, as described in U.S. published patent application 2007/0077274 to Ahlers, entitled “Methods for Producing Shaped Bodies Based on Crosslinked Gelatin,” incorporated herein by reference. See also, U.S. Pat. No. 5,954,748 to Todakura, entitled “Gelatin Coated Gut Suture,” incorporated herein by reference. Chemical crosslinking of collagenous material long been used to form long lasting prosthetic materials, such as crosslinked bovine pericardium used for heart valve prostheses. Gelatin, a collagen derivative, can be similarly crosslinked for long term implantation. See, for example, published U.S. patent application 2021/0322154 to Tian, entitled “Method of Processing Collagen-Based Tissue for Bioprosthetic Devices,” incorporated herein by reference.

The hydrogel precursors include at least one electrophile composition and one nucleophile composition dissolved into the solvent. Each precursor generally has a plurality of functional groups, such as 3 or more, to provide for crosslinking. The separate precursors can be provided as a fine powder for forming the flowable composite composition upon dissolving of the powders. The fine particles of the precursors generally have an average particle diameter of no more than about 5 mm. In general, the precursors dissolve readily, and particle sizes are not particularly significant. The powders of the different precursor compositions can be provided as a mixed powder or separate precursor powders. The hydrogel precursors can optionally comprise a plurality of electrophiles and/or a plurality of nucleophiles. The hydrogel precursors can be designed to have roughly equal amounts of electrophilic functional groups and nucleophilic functional groups so that full crosslinking can occur and is expected to occur between the hydrogel precursors without any precursors necessarily bonding to functional groups in blood or tissue. In some embodiments, the ratio of electrophilic groups and nucleophilic groups, such as amine groups protonated for delivery, can deviate from 1, for example, in a molar ratio range of about 1:0.5 to 1:1.5, in some embodiment in a range from about 1:0.75 to about 1:1.25, in other embodiments form about 1:0.9 to about 1:1.1, and in some embodiment a molar ratio range of about 1:1.05 to about 1:1.25 or in a molar ratio of about 0.75:1 to about 0.95:1, to provide a different crosslink density and correspondingly potentially different mechanical properties and/or gel times. A person of ordinary skill in the art will recognize that additional ranges of electrophile and nucleophile groups within the explicit ranges above are contemplated and are within the present disclosure.

The crosslinking of the hydrogel precursors is gated by pH. In particular, protonation of nucleophilic groups, generally amines, blocks their ability to perform nucleophilic substitution resulting in the crosslinking. Biological tissue and bodily fluids, such as blood, have an alkaline pH which is carefully buffered, generally between 7.32 and 7.42 pH units. After deposition of the flowable composite composition, contact with bodily fluids results in a raising of the pH, deprotonation of the nucleophilic functional groups, for example an amine, and initiation of the hydrogel crosslinking reaction leading to gelation. To avoid delay in the gelling process, the flowable composite composition can be free of any added buffer since any buffer can slow adjustment of the pH due to contact with bodily fluid. Of course, since all of the materials are exposed to atmosphere, the materials have some dissolved carbon dioxide, which tends to lower the pH slightly through formation of carbonic acid. Of course, bodily fluids also comprise dissolved carbon dioxide, and the buffering of bodily fluids accounts for the effects of dissolved carbon dioxide. While all of the exemplified embodiments are free of any added buffer, the presence of small amounts of buffer may not significantly influence the gelation, and some additives could provide some minor buffering effect.

The nucleophilic precursor is generally supplied in protonated form. Since HCl is a convenient strong acid that is generally used to perform the protonation, the composition may be indicated with a notation of (• HCl) and with an understanding that the proton is bound to the functional group, such as —NH₃⁺ and the chloride ion (Cl⁻) as a spectator ion. The powder of the precursor comprises the protonated form, which can be referred to as salted based on the protonation of the amine and the presence of the chloride ion. Once the precursors are hydrated, the degree of protonation depends on the pKₐ of the protonated functional group since an equilibrium becomes established in the solvent with the dissolved precursors. The exemplified PEG-amines have a suitable pKₐ to maintain protonation in saline while providing rapid deprotonation and initiation of crosslinking upon contact with bodily fluids. The pKₐ of amines depends on the organic moiety bound to the amine. Medical saline, water for injection and similar solvents for forming the precursor solution generally are acidic with a pH near 5.5 due primarily to dissolved carbon dioxide. The natural acidity of the solvent helps to maintain the amine protonation until the pH is raised by contact with bodily fluids. The buffer capacity resulting from dissolved saline is basically negligible (0.126-0.152 mEq./L), although not quite zero. See, Reddi, “Why is Saline so Acidic (And Does it Really Matter?),” International Journal of Medical Science, Vol. 10(6), 747-750 (April 2013).

With respect to buffers, the hydrogel precursors are not considered to be buffers whether or not they alter the pH and may, in principle, provide some buffer function. In general, dissolved carbon dioxide is present, and this is not considered an added buffer and, as noted in the previous paragraph, carbon dioxide provides negligible buffer capacity. The amine precursors are provided in an acidified form with the acidic proton functioning as a protecting group blocking crosslinking. Of course, the acidification is in equilibrium in solution. Upon contact with a bodily fluid at physiological pH, the acidified amine can be neutralized so that it can crosslink at an appropriate rate with the electrophilic precursor. The precursor solution generally should have no added buffer capacity or an appropriate buffer capacity. A buffer can be considered as any Brønsted base, which generally would be an anion (B⁻) corresponding to a weak acid (HB), in equilibrium with the corresponding weak acid. Anions corresponding to strong acids, such as halide anions, do not act as buffers. If the precursor solution has added buffer capacity, it can shorten the pot life and/or lengthen the gelation time, but such effects can be within tolerable ranges. Effects of buffers depend on the pKₐ and the concentration. While it is generally desirable to have no added buffer apart from impurities and a dissolved carbon dioxide contribution in the precursor solution, appropriate concentrations of appropriate added buffer would generally be acceptable for at least some applications. Due to these complexities, a suitable buffer and concentration can be evaluated based on storage stability and gel times. As a reference point, the buffer capacity approximately provided by dissolved atmospheric carbon dioxide is very small at about 0.126-0.152 milliequivalents/liter/pH (mEq/L/pH), see Reddie, “Why is Saline do Acidic (And Does it Really Matter?). International Journal of medical Sciences, 10(6), 747-750 (2013). While the extremely low buffer capacity of saline does not seem detrimental to flowable composite composition performance, distilled water can be used to further lower the buffer capacity to roughly zero. At the other end, another reference point is the buffer capacity of blood (38.5 mEq/L/pH) and blood plasma (16.1 mEq/L/pH). A buffer capacity of no more than about 2 mEq/L/pH generally should result in little if any detrimental change of gelling properties, and generally the flowable composite composition can have a buffer capacity in some embodiments from about 0 to about 1.5 mEq/L/pH, in further embodiments from about 0.01 mEq/L/pH to about 1.25 mEq/L/pH and in some embodiments from about 0.05 mEq/L/pH to about 1 mEq/L/pH. A person of ordinary skill in the art will recognize that additional ranges of buffer capacity within the explicit ranges above are contemplated and are within the present disclosure.

With respect to the hydrogel precursors, suitable functional groups are known for crosslinking monomers to form gelled hydrogels in situ. Suitable monomers, generally macromers as specified below, can comprise electrophilic groups that demonstrate activity toward amine functional groups with respect to addition reactions that result in the desired crosslinking. Thus, multi-component hydrogel systems can spontaneously crosslink when the components are activated by contact with physiological liquids, but the two or more components are appropriately stable for a reasonable process time before activation by the physiological liquids. Such systems include, for example, monomers (generally, although not necessarily, macromers) that are di- or multifunctional number of amines in one component and macromers with di- or multifunctional number electrophilic groups, such as N-hydroxysuccinimide ester containing moieties, in the other component. N-hydroxysuccinimide ester functional groups facilitate amide bond formation in reactions with amines and have been used in other medical hydrogels, although other suitable electrophilic precursors are described below. The N-hydroxysuccinimide esters are generally pendent on a hydrophilic core.

The hydrogel precursors can have crosslinking activated by the physiological fluids contacted by the precursors following delivery. The hydrogel precursors described herein can be designed to hydrate relatively quickly. Hydrogel and precursor solution properties are described further below. The parameters that influence the properties include functional group chemistry, crosslinking density/molecular weights of the monomers, monomer composition, and concentrations of the granular component and the precursors.

The crosslinking density of the resultant biocompatible crosslinked hydrogel is controlled by the overall molecular weight of the macromers and the number of functional groups available per molecule. A lower molecular weight between crosslinks such as 600 Da give a higher crosslinking density as compared to a higher molecular weight such as 10,000 Da. Higher molecular weight macromers with significant branching provide desirable gelatin times, and in some embodiments more than 2500 Da so as to obtain elastic gels. In certain embodiments, the nucleophilic acid conjugated precursor molecules (with an amine) is not significantly smaller in molecular weight than the electrophilic precursor molecules. In some embodiments, it is the same size or larger.

The crosslinking density also may be controlled by the ratio of nucleophilic groups and electrophilic groups in the mixed precursor material. Following delivery of the flowable composite composition, the gel times depend significantly on the time to raise the pH due to interaction with bodily fluids since the amines deprotonate to be available for nucleophilic substitution. While not wanting to be limited by theory, it is thought that longer gel times can be related to slower diffusion of physiological fluids into and/or slower diffusion of the acid conjugate species out of in situ placed composition. From the perspective of the flowable composite composition, the configuration of the deposited composition can influence the gel time since a thicker deposit can establish a longer diffusion path for the raising of the pH to deprotonate the amine group to provide for crosslinking. As noted below, prior to and/or after delivery of the flowable composite composition, a crosslinking accelerator solution can be added to stimulate the crosslinking reaction.

Yet another method to control crosslink density is by adjusting the stoichiometry of nucleophilic functional groups to electrophilic functional groups. A one-to-one ratio of electrophilic groups and amine groups should provide for a highest crosslinking density, although electrophilic groups in the precursors can react, in principle, with amines in proteins in physiological solutions as well as in the tissue, or may remain unreacted. In the Examples, desirable performance is obtained with a 1:1 ratio of functional groups, but alternative ranges are discussed below. Cured compositions with unreacted functional groups can provide tackiness to facilitate adhesion, which along with the cohesion provided by the crosslinking, can improve function of the sealing composition.

### Monomers—Hydrogel Precursors

In this section, the hydrogel precursors are referred to as monomers to emphasize their role in composite system and in particular, the crosslinked hydrogel. Monomers capable of being crosslinked to form a biocompatible structure, e.g., a sealant deposit, may be used. Electrophilic hydrogel precursor and nucleophilic hydrogel precursor generally are solids at room temperature, but in principle, at least one precursor can be a room temperature liquid. For one or both precursors, monomers can be macromers, which may or may not be polymers. The term polymer, as used herein, means a molecule formed of at least three repeating groups, which then may have reactive functional groups pendent on the polymer. Generally, the term “reactive precursor species” means a polymer, functional polymer, macromolecule, or small molecule that can take part in a reaction to form a network of crosslinked molecules, e.g., a hydrogel. As noted above, for the formation of rapidly crosslinking hydrogel precursor systems, the monomers are generally, although not necessarily, macromers, as specified below since macromers generally allow for more rapid hydration along with more rapid deprotonation of the amines.

Monomers may include, for example, the biodegradable, water-soluble macromers described in U.S. Pat. No. 7,332,566 to Pathak et al. (hereinafter the '566 patent), entitled “Biocompatible Crosslinked Polymers With Visualization Agents,” incorporated herein by reference. These monomers are characterized by having at least two polymerizable groups, which may or may not be separated by at least one degradable region. Upon crosslinking, the product polymers form coherent hydrogels that persist indefinitely or until eliminated by degradation, which can involve, for example, enzymatic reactions or hydrolysis. Generally, a macromer is formed with a core of a polymer that is water soluble and biocompatible, such as a polyalkylene oxide, e.g. polyethylene glycol, which can be flanked by hydroxy-carboxylic acids such as lactic acid, to form degradable esters or non-degradable amides. Suitable monomers, in addition to being biocompatible, and non-toxic, also can be at least somewhat elastic after crosslinking or curing. For the electrophilic compounds or compounds with amine groups, the cores of the compounds can have a plurality of arms or branches each with a functional group suitable for crosslinking. PEG-based polymers with three or more arms are generally star polymers with a branched core with the extending PEG polymer arms. As noted above polyethylene glycol (PEG) based monomers are established medical hydrogel precursors, and precursor compounds are commercially available with various numbers of arms, molecular weights and functional groups.

The nucleophilic functional groups generally are amine groups. The amine groups can be protonated as a protecting group or gate to control crosslinking. The nucleophilic amine groups of the precursors can be designed to significantly deprotonate at physiological pH values, such as from about 7.1 to about 7.6 pH units, although blood and tissue generally is at a narrower pH range in a healthy individual. While macromers are exemplified and provide desirable properties, trilysine has been used in medical hydrogels as a poly-amine monomer, and similar compounds can be used. Thus, in some embodiments, smaller molecular polyfunctional amines can be used as nucleophiles, such as trilysine, dilysine, tetralysine, mixtures thereof or the like. The electrophilic functional groups can be selected to react in addition reactions with the amines to form crosslinks. N-hydroxylsuccinimide esters are desirable electrophilic groups, although other suitable groups are described below. One or both of the functional groups can be pendent on a hydrophilic core. Since the precursor composite composition is pre-hydrated, the deposited composite material can be designed to have little or no swelling, which can be desirable from the perspective of conformal filling, while avoiding stressing of the wound. In some embodiments, the polymers may have a hydrolytically biodegradable portion or linkage, for example an ester, carbonate, or other suitable linkage, although enzymatically degradable linkages may be present additionally or alternatively. Several such linkages are well known in the art and originate from alpha-hydroxy acids, their cyclic dimers (anhydrides), or other chemical species used to synthesize biodegradable moieties, such as, glycolide, dl-lactide, l-lactide, caprolactone, dioxanone, trimethylene carbonate or a copolymer thereof. In particular, the electrophilic monomers can conveniently be provided with degradable linkages.

In some embodiments, the monomer providing electrophilic functional groups and the monomer providing the amine groups are macromers to provide for desirable hydration of the precursors upon contact with the bodily fluids. The macromers generally have biologically inert and water soluble cores with pendent reactive functional groups for crosslinking. When the core is a polymeric region that is water soluble, polymers that may be used can be natural or synthetic polymers. Suitable polymers for the core can include polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, poloxamers, such as Pluronic® F-127; as well as polyoxazolines, polyvinyl alcohol (“PVA”); poly(vinyl pyrrolidinone) (“PVP”); and polysaccharides, such as hyaluronic acid, chitosan, dextran or digested cellulose and derivatives thereof. Based on extensive experience in existing medical products, star-branched polyethers and more particularly polyethylene glycol (also known as poly(oxyalkylenes) or poly(ethylene oxide)) are especially suitable. Acidified amines or electrophilic groups can be located at the end of the arms of each branch or a portion thereof. For PEG precursors, a common notation in the medical hydrogel art is to refer to the number of arms and molecular weight along with the functional groups on the arms, such as 4A15K NH₂—HCl for a 4 arm PEG with a 15,000 Daltons molecular weight and acidified amine with chloride ions or 8A20K NHS ester, for an eight arm PEG with a molecular weight of 20,000 Daltons with a N-hydroxysuccinimidyl ester functional group.

PEG based hydrogels have found broad use in medical products. As a result, they are widely accepted and PEG-monomers with a range of functionality are commercially available in medical grade. Polyoxazolines have gained attention as a potentially desirable alternative to PEG-based products. Poly(2-oxazoline)s have the structure —(CH₂CH₂N(COR))—, where the R group can be H, an alkyl group or other functionality. Amine terminated poly(2-ethyl-2-oxazoline) is available from Sigma-Aldrich. Terminally functional monomers do not allow for crosslinking, but a polyfunctionalized electrophilic monomer with three or more functional groups can effectuate crosslinking. Poly(2-R-2-oxazoline)s with 25% side chains populated with N-hydroxylsuccinimiide (NHS)-ethyl groups were synthesized as described in published U.S. patent application 2019/0125922 to Bender et al., entitled “Tissue-Adhesive Porous Hemostatic Product,” incorporated herein by reference.

It has been determined that hydrogels formed with macromers with longer distances between crosslinks are generally softer, more compliant, and more elastic. Furthermore, the crosslink density can influence swelling. The crosslinking density of the resultant crosslinked hydrogel polymer can be controlled by the overall molecular weight of the precursors and the number of functional groups available per molecule. A lower molecular weight between crosslinks such as 600 Da will give much higher crosslinking density as compared to a higher molecular weight such as 10,000 Da. Another parameter to control crosslink density is the stoichiometric ratio of nucleophilic functional groups to electrophilic functional groups. A one to one ratio generally leads to the highest crosslink density. A larger distance between crosslinks can also contribute to greater swelling, and a shorter distance between crosslinks can be useful to form a hydrogel with little or no swelling. Thus, in the polymers of the '566 patent, increased length of the water-soluble segment, such as polyethylene glycol, tends to enhance elasticity. Molecular weights of hydrophilic macromers as used herein, such as macromers with polyethylene glycol macromer cores, generally are at least about 2,000 Da, in some embodiments from about 2500 Da to about 500,000 Da, in other embodiments from about 5,000 Da to about 250,000 Da, in further embodiments from about 7500 Da to about 100,000 Da, in additional embodiments from about 10,000 Da to about 50,000 Da, and in other embodiments in the range of about 15,000 Da to about 40,000 Da. PEG precursors in the lower portions of these weight ranges may be liquids. As used herein, molecular weights (mass) are in conventional units, which can be equivalently Daltons or as a molar mass-grams/mole (assuming natural isotopic presence in either case), and for polymers molecular weights are generally reported as averages if there is any distribution of molecular weights. A person of ordinary skill in the art will recognize that additional ranges within the explicit ranges above are contemplated and are in the present disclosure.

The hydrogel precursors in the hydrogel precursor solutions have a ratio of electrophilic functional groups and amine functional groups. The ratio of functional groups can alter the crosslinking density and the nature of the resulting hydrogel. Generally, if the number ratio of electrophilic functional groups to amines is one to one, the hydrogel can fully crosslink given sufficient time and lack of constraints. As noted above, a ratio of functional groups different from 1 may result in a composite sealing material that is somewhat tacky with respect to adhesive ability, possibly due to enhanced van der Waals forces, although not wanting to be limited by theory. Generally, the ratio of electrophilic functional groups to nucleophilic functional groups can be from about 0.5 to 1.5, and in further embodiments from about 0.75 to about 1.25, in further embodiments from about 0.80 to about 1.20, in other embodiments from about 0.85 to about 1.15, in additional embodiments from about 0.90 to about 1.1 and in some embodiments from about 0.95 to about 1.05, although the ratio can be approximately 1.0. Person of ordinary skill in the art will recognize that additional ranges of ratios within the explicit ranges above are contemplated and are within the present disclosure.

To achieve the desired ratios of functional groups, the functional groups can be distributed in various ways. Pendent functional groups extending from a core can be referred to as being associated with an arm of the precursor. A precursor generally has 2, 3, 4, 5, 6, 7, 8, 9, 10 or more arms. At least one precursor generally has at least 3 arms to obtain crosslinking, and 4, 6 or 8 armed precursors can be convenient for obtaining desirable hydrogel properties. To obtain a one-to-one ratio of functional groups, equal molar amounts of precursors can be used if they have the same number of arms, or if different numbers of arms are present on the respective precursors, the mole ratios can be correspondingly adjusted. Thus, twice the molar amount of a 4-arm precursor can be combined with an 8-arm precursor to obtain a 1:1 functional group ratio. For weight ratios, the mole ratios can be adjusted accordingly based on the relative weights, for an 8-arm 10K MW (10,000 molecular weight) precursor it would be combined with twice the mass of an 8-arm 20K MW precursor to get a 1:1 functional group ratio. A person of ordinary skill in the art can adjust these calculations to obtain a different number ratio for the functional groups.

### Functional Groups and Crosslinking Reactions

The crosslinking reactions generally are designed to occur upon interaction with physiological fluids in vivo, surrounded by physiological conditions, although the medical procedure may involve some local dilution or modification of the physiological fluids, such as with disinfecting agents or other procedural expedient, from their pure natural state without changing the basic processing of the hydrogel precursors. Unless specifically stated otherwise, reference herein to physiological fluids can involve minor modifications of natural fluids due to the medical procedure. Thus, the crosslinking reactions occur “in situ”, meaning they occur at local sites such as on an organ or tissues in a living animal or human body. Due to the in situ nature of the reaction, the crosslinking reactions can be designed not to release undesirable amounts of heat of polymerization. Gelation times for desirable procedures are described above, and full crosslinking can be completed generally after times from 2 minutes to 10 hours, although other times outside this range may be acceptable. For longer complete crosslinking times, the times may begin to compete with degradation times.

Certain functional groups, such as alcohols or carboxylic acids, do not normally react with other functional groups, such as amines, under physiologically acceptable pH. However, such functional groups can be made more reactive by using an activating group such as N-hydroxysuccinimide or derivatives thereof. In general, several methods for activating such functional groups are known in the art. Suitable activating groups include, for example, carbonyldiimidazole, sulfonyl chloride, chlorocarbonates, aryl halides, sulfosuccinimidyl esters, N-hydroxysuccinimidyl ester (NHS), succinimidyl ester, succinimidyl amide, epoxide, aldehyde, maleimides, imidoesters and the like. The N-hydroxysuccinimide esters or N-hydroxysulfosuccinimide groups are desirable groups for crosslinking of amine functionalized polymers such as amino terminated polyethylene glycol (“APEG”) since they have found acceptance in medical implants from long periods of use in approved products. A further extensive discussion of general medical hydrogels is found in the '566 patent cited above.

Suitable nucleophilic functional groups are polymers with primary amines. Thus, the other functional group used for the crosslinking with electrophiles generally is an amine. Amines are weak bases. In some embodiments, the acid conjugate is HCl, and an HCl salted amine, such as a PEG amine, is formed. The acid conjugate used to protonate the amine can be chosen to match the approximate molarity of the amine. The advantage of an NHS-amine reaction is that the reaction kinetics lead to quick gelation usually within 10 about minutes, in some embodiments within about 1 minute and in further embodiments less than 30 seconds. The protonated amines are generally not suitable for performing nucleophilic substitution until deprotonated by contact with an alkaline bodily fluid.

The crosslinking density of the resultant biocompatible crosslinked hydrogel is controlled by the overall molecular weight of the monomers and the number of functional groups available per molecule. A lower molecular weight between crosslinks such as 2000 Da give a higher crosslinking density as compared to a higher molecular weight such as 100,000 Da. Higher molecular weight monomers can be used to obtain more elastic hydrogels, and correspondingly lower molecular weight monomers can be used to obtain less elastic hydrogels. Different applications may suggest different properties for the hydrogels.

Another method to control crosslink density is by adjusting the stoichiometry of nucleophilic functional groups to electrophilic functional groups. A one to one ratio can lead to the highest crosslink density. In general, over time, the hydrogel completes curing so that available crosslinking sites form crosslinking bonds. If the electrophilic and nucleophilic functional groups are provided in equivalent amounts it can be expected that approximately all functional groups form crosslinking bonds after full curing. Equal numbers (or reaction equivalents) of the two types of functional groups generally provides the highest crosslinking density. If a different ratio of functional groups is used, the properties of the cured hydrogel can be accordingly somewhat different. The crosslinking density can depend on the number of functional groups on the precursor molecules as well as on the ratio of precursor molecules. A non-stoichiometric ratio of electrophilic and nucleophilic groups can be used to alter the crosslinking density if desired. The ratio of functional groups is described further above.

### Degradable or Non-Degradable Linkages

For certain embodiments, it is desirable for the cured composite material to be degradable, and in some embodiments relatively quickly degradable. Thus, if the cured composite compositions are in contact with tissue, they do not persist indefinitely. For the composite compositions to degrade, both the granular component and the in situ formed hydrogel degrade. But in alternative embodiments, the cured compositions can be long lasting and potentially essentially permanent. Thus, depending on the application, it may or may not be desirable for the hydrogel to be degradable, such as through hydrolysis or biodegradation due to enzymatic activity, although for hemostatic applications, the composite hydrogel compositions generally are designed to degrade quickly so they do not persist long after stable clotting. If it is desired that the biocompatible crosslinked hydrogel polymer be degradable or absorbable, one or more precursors having degradable linkages present in between the functional groups may be used. Similarly, crosslinking of the granular compositions can provide inhibition of degradation. As used in the art, absorbable polymers can be referred to as biodegradable if they are absorbed under physiological conditions, whether or not they degrade by biological action, such as enzymatic cleavage. The degradable linkage optionally also may serve as part of the water soluble core of one or more of the precursors. In the alternative, or in addition, the functional groups of the precursors may be chosen such that the product of the reaction between them results in a degradable linkage. For each approach, degradable linkages may be chosen such that the resulting degradable biocompatible crosslinked hydrogel polymer degrades or is absorbed in a desired period-of-time range. In other embodiments, functional groups and linkages with functional groups can be selected to resist degradation under physiological conditions to substantially reduce or eliminate absorption of the cured composite compositions.

Generally, degradable linkages are selected that degrade the hydrogels under physiological conditions into non-toxic products for removal from the patient by natural pathways. Illustrative enzymatically hydrolyzable biodegradable linkages include peptidic linkages cleavable by metalloproteinases or collagenases. Additional illustrative biodegradable linkages can be functional groups on the core polymers and copolymers, such as hydroxy-carboxylic acids, orthocarbonates, anhydrides, lactones, (aminoacids, carbonates, phosphonates or combinations thereof. In exemplified embodiments, the degradable linkages are esters formed by hydroxy-carboxylic acid moieties adjacent the electrophilic group used for crosslinking. Esters can degrade gradually by hydrolysis under physiological conditions, with persistence time depending on the specific structure. Granular materials may be degradable by hydrolysis, enzymatic effects or both. To have non-degradable hydrogels, the esters formed by hydroxy-carboxylic acid moieties can be replaced by amide groups that generally do not hydrolyze under physiological conditions. Monomers with PEG cores are commercially available with N-hydroxysuccinimide electrophilic groups attached with amide linkages or alternatively with ester linkages, for example, from Jenkem Technology, TX, U.S.A. PEG-amines are also available from Jenkem with various numbers of arms and molecular weights. Desirable degradable electrophilic groups include, for example, N-hydroxy succinimidyl succinate (SS), N-hydroxy sulfosuccinimidyl succinate, N-hydroxy sulfosuccinimidyl gluterate, succinimidyl glutarate (SG), succinimidyl adipate (SAP), succinimidyl azelate (SAZ), or a mixture thereof. Examples are described below of rapidly degrading patches with the SS linker. Mixtures of degradable linkages and non-degradable linkages, such as the amides described above, can be used to adjust the persistence time, such as to form oligomeric species for clearing by the body.

As noted above, thermally crosslinked gelatin can be effective for selective degradation times with the degree of crosslinking providing a parameter to adjust the degradation times. Other materials can be appropriately designed for degradation. Gelatin and collagen can be chemically crosslinked to make the granular composition resistant to degradation. Collagenous tissue has been chemically crosslinked for use in implanted prosthetic devices for long term use for decades. For example, see U.S. Pat. No. 4,050,893 to Hancock et al., entitled “Arrangement for Preparing Natural Tissue for Implantation,” incorporated herein by reference. This chemical crosslinking can be referred to as tanning, based on historical relationship to tanning of hides. Tanning agents generally involve glutaraldehyde, but formaldehyde can be used and other less toxic agents have been proposed. While the crosslinking reaction can be reversible, resulting in release of toxic glutaraldehyde, proper longer term crosslinking results in a stable, irreversible structure due to the formation of extensive crosslinked networks. Nevertheless, some toxicity can remain, and chemical treatments can be used to reduce the toxicity. See U.S. published U.S. patent application 2003/0130746A to Ashworth et al., entitled “Biocompatible Prosthetic Tissue,” incorporated herein by reference.

### Additives/Visualization Agents

The flowable composite composition can comprise additives in addition to solvent, granular hydrophilic composition and the hydrogel precursors. In particular, the compositions can comprise a visualization agent, which can provide visual and/or imaging modality-based visualization. Furthermore, other suitable additives can include, for example, a viscosity modifier or thickener, a friction reducing agent to facilitate injection, or a mixture thereof. A viscosity modifier can comprise for examples a thickener, such as soluble starch, hyaluronic acid or derivatives thereof, soluble polypeptides, polyethylene glycol, mixtures thereof, or the like. A friction reducing agent can comprise fatty acids or the like. Generally, the total additives comprise no more than about 5 wt % of the flowable composite composition, in some embodiments from about 0.0001 wt % to about 4 wt % and in further embodiments form about 0.001 wt % to about 3 wt %. A person of ordinary skill in the art will recognize that additional ranges of additive concentrations within the explicit ranges above are contemplated and are within the present disclosure. The compositions can further comprise therapeutic agents, and therapeutic agents are described below. In general, any suitable therapeutic agent can be used as long as compatible with the hydrogel precursors.

As noted above, the flowable composite composition and the gelled composite composition can comprise a visualization agent to assist with visualization of the composition during a medical procedure. As used herein, visualization agents can refer to optical visualization (with color), or visualization using an imaging modality, such as x-ray or ultrasound. Visualization agents are especially useful when used in minimally invasive surgery (MIS, e.g., laparoscopy) procedures, due among other reasons to their improved visibility on a color monitor. It is sometimes useful to provide color by adding a colored visualization agent to the flowable composite composition for delivery.

Visualization agents (optical) may be selected from among any of the various non-toxic colored substances suitable for use in medical implantable medical devices, such as FD&C BLUE dyes 1, 2, 3 and 6, indocyanine green, or colored dyes normally found in synthetic surgical sutures. In some embodiments, green or blue colors are desirable because these have better visibility in presence of blood or on a pink or white tissue background. While visualization aids are generally applicable to assist with proper placement for medical procedures, visualization agents can also be selected based on aesthetic reasons, especially for applications in which a long lasting or effectively permanent hydrogel composite is used.

For dyes used as visualization agents, these are generally water-soluble. Water-soluble dyes can be expected to wash out of the hydrogels relatively quickly. If the dyes have or are modified to have appropriate functional groups, the dyes can be bound to functional groups of the hydrogel precursors, which can be performed prior to crosslinking the hydrogel or simultaneously with crosslinking the hydrogel. Suitable functional groups include for example, amines (—NH₂, carboxylates (—COO—), thiols (—SH), or other appropriate electrophilic or nucleophilic groups. Carboxylates can be reacted to form —NHS groups similarly to the precursor compounds. With a single functional group, the dye would not crosslink, but with covalent bonding, the dye would be expected to persist until the hydrogel degrades and is resorbed by the patient. Fluorescein-NHS is commercially available as a fluorescent red/orange dye. A set of dyes can be used for image generation if desired, to cover the primary colors, or alternative colors or shades.

The visualization agent may be present with the precursor solution prior to delivery of the flowable composite composition. The selected colored substance may or may not become chemically bound to the crosslinking hydrogel. Additional visualization agents may be used, such as fluorescent (e.g., green or yellow fluorescent under visible light) compounds (e.g., fluorescein or eosin), x-ray contrast agents (e.g., iodinated compounds) for visibility under x-ray imaging equipment, ultrasonic contrast agents (e.g., microbubbles), or MRI contrast agents (e.g., Gadolinium containing compounds). Biocompatible visualization agents FD&C BLUE #1 and fluorescein-NHS can be particularly desirable for some applications.

As noted above, visually observable visualization agents can be advantageously used for some embodiments. Wavelengths of light from about 400 to 750 nm are observable to the human as colors (R. K. Hobbie, Intermediate Physics for Medicine and Biology, 2^{nd} Ed., pages 371-373). Blue color is perceived when the eye receives light that is predominantly from about 450 to 500 nm in wavelength and green is perceived at about 500 to 570 nm (Id.). Further, since the eye detects red or green or blue, a combination of these colors may be used to simulate any other color merely by causing the eye to receive the proportion of red, green, and blue that is perceived as the desired color by the human eye. The color blue, as used herein, means the color that is perceived by a normal human eye stimulated by a wavelength of about 450 to 500 nm and the color green, as used herein, means the color that is perceived by a normal human eye stimulated by a wavelength of about 500 to 570 nm.

In some embodiments, one or more visualization agents can be present in the flowable composite composition comprising the precursor solution at a concentration of no more than about 0.1 weight percent (wt %) and in some embodiments in a concentration range of at least 0.001 to about 0.075 wt %, and in further embodiments in the range of 0.0025 to 0.05 wt %, although greater concentrations may potentially be used, up to the limit of solubility of the visualization agent and selected concentrations may be influenced by the particular visualization agent and the particular application. In some applications, these concentration ranges were found to give a color to the hydrogel that was desirable without interfering with crosslinking times (as measured by the time for the reactive precursor species to gel). The visualization agent is generally not covalently linked to the hydrogel. A person of ordinary skill in the art will recognize that additional ranges of visualization agent concentrations within the explicit ranges above are contemplated and are within the present disclosure.

In some embodiments, a visualization agent based on use of an imaging modality to provide visualization is contemplated. For example, iodine atoms can be derivatized onto the hydrogel precursors to provide enhanced x-ray imaging capability. The use of PEG-based precursors with iodine labeling is described further in U.S. Pat. No. 8,383,161 to Campbell et al., entitled “Radioopaque Covalently Crosslinked Hydrogel Particle Implants,” incorporated herein by reference. These hydrogels can have a Hounsfield number of at least about 90. In addition, the composite sealing material can be made echolucent. Due to the viscosity of the precursor composite material, if the precursor is sonicated shortly prior to injection, air bubbles can be maintained in the composition for a reasonable period of time can become entrapped. Alternatively, commercially available ultrasound contrast media with gas filled microbubbles can be blended with the precursor composite composition. Microbubbles can be formed from gas, such as air or CO₂, within a shell, such as a lipid. The microbubbles can dissipate over a reasonable period of time, such as hours or longer into harmless components. Commercial echolucent microspheres are sold as ultrasound contrast agents, such as Definity® (Perflutren lipid microspheres in liquid PEG, Lantheus), Optison™ (Perflutren Protein Microspheres, PEG-free, GE Healthcare), Sonazoid® (perfluorobutane microbubbles, GE Healthcare) and SonoVue® (Bracco). An appropriate quantity of microspheres can be blended into the precursor composite composition to provide enhanced ultrasound visualization without significant altering desirable mechanical properties. The image enhancing microspheres can be concentrated relative to the injectable products for incorporation into the composite sealant.

In some embodiments, a method is used to form a hydrogel on a tissue until the color of the hydrogel indicates that a predetermined volume of hydrogel has been deposited on the tissue or within the space. The precursors are continually introduced into the space until the color of the materials that enter that space and flow out are deemed to have achieved a suitable content, as indicated by observation of the visualization agent disposed in the materials that flow out. In this way, any bodily fluids, such as blood, can be cleared from the wound by the initial delivery of the precursor composite composition, and observation can confirm that the wound is sufficiently cleared for sealing.

The visualization agent may serve to help visualize the injection of the hydrogel in specific cases. Further, the hue of a colored hydrogel may provide information about the concentration of the precursors in the hydrogel or the degree of mixing of physiological fluids into the hydrogel. A dark color hydrogel may indicate a concentration of precursors that is high relative to a lighter hued hydrogel made from the same precursor solutions. The coloring agent may be provided in a preselected amount for the application and may be part of a kit, such as kits described further below. The visualization agent may be a powder that is dissolved into the solvent at an appropriate point in preparing the flowable composite composition. In some embodiments, the dye is conjugated to an electrophilic or nucleophilic end group to allow for incorporation into the gelled material for visualization with direct correlation to persistence. In some cases, the dye is fluorescent, allowing for visualization under special lighting conditions only and render the cured composite material otherwise invisible under normal visual conditions.

In some embodiments, a medical professional can apply a flowable composite composition to a tissue location with a selected visualization agent to observe directly the injectable composition, as described in the context of the specific Examples. The user may use visualization agents to see the flowable composite composition with the human eye or with the aid of an imaging device that detects visually observable visualization agents, e.g., a video camera. A visually observable visualization agent is an agent that has a color detectable by a human eye. An alternative embodiment is a visualization agent that may not normally be seem by the human eye but is detectable at a different wavelength, e.g., the infrared or ultraviolet, when used in combination with a suitable imaging device, e.g., a video camera. Hydrogels with visualization agents for x-ray and/or ultrasound visualization are described further in U.S. Pat. No. 8,383,161 to Campbell et al., entitled “Radioopaque Covalently Crosslinked Hydrogel Particle Implants,” incorporated herein by reference. Ultrasound visualization can be particularly desirable for laparoscopic procedures, and x-ray visualization can be particularly desirable for tumor delivery.

Properties of Flowable Composite Composition with Hydrogel Precursors, Cured Composite Material and the Curing Process

The composition of the flowable and cured composites are described above. The compositions are designed to provide desirable properties that underlay their functions. The flowable composite compositions are designed to be flowable, yet stay in place at the application location for gelling, conformally fill wounds or other location defects, achieve rapid gelling and form a good sealant, which may be hemostatic with respect to significant bleeds. The cured material can have good mechanical stability, while providing a cushioned interface with tissue, maintenance of a good seal, provide a good environment for healing, and in some embodiments, being resorbable in a desired period of time. The flowable composite composition can be delivered through a convenient needle or cannula, and pressure can be applied with an appropriate material, such as a gauze, to facilitate formation of a good seal. Upon contact with moist tissue, blood or other bodily fluids, gelation should initiate based on a raising of the pH. The resulting chemical crosslinking results in the formation of a non-flowable, integral composite material. The composite material forms a seal that is resorbed gradually while the tissue heals.

As noted above, the flowable composite composition can be characterized by injectability, viscosity, cohesion, storage time and gel time with respect to delivery and maintaining its presence at the delivery location, as well as gel time and ability to form a rapid seal, and potentially hemostasis. For foamed embodiments, the flowable composite composition can be further characterized by properties of the foam. One way to characterize foam is through the density that is a function of the relative volume of gas/air in the foam. In principle, flowable foam can be characterized by rheology, and by analogy with liquids, a foam can be said to have generally non-Newtonian rheology. As such, the flowable foam can resist flow in static placement, but can flow on the application of shear during injection. In contrast, a solid foam fractures on application of enough shear, which may be a process used to form the granular material in the composite. Injectability of the composite precursor composition is generally determined by the granule size, viscosity, and injection tool. The granule size should be selected to be smaller than orifices encountered in the injection tool, although the hydrated granules may be flexible. Thus, for a syringe, the granules should be smaller than the needle/cannula inner diameters by a reasonable amount as well as smaller than any orifices at connectors or the like. Compressibility and flexibility of the particles provides some extra cushion for slightly larger particles, especially in lower amounts. An atomizer orifice may not have a large axial extent, so the deformability of hydrated granules may facilitate passage through an atomizer. Generally, for embodiments involving a catheter, the granules should have the largest diameters of about 0.65 times the cannula inner diameter, in some embodiments no more than about 0.50 times the inner diameter and in further embodiments no more than about 0.33 times the inner cannula diameter. For foamed embodiments, the foam density generally ranges from about 30% to about 98%, in further embodiments from about 35% to about 95% and in other embodiments from about 40% to about 90% of the density of corresponding unfoamed composition. The density of an unfoamed composition can be evaluated based on the volume of solvent and the weights of the components added to the solvent. The foam density can be selected through adjustment of the volume of entrapped gas/air. A person of ordinary skill in the art will recognize that additional ranges of largest particle diameters and relative foam density within the explicit ranges above are contemplated and are within the present disclosure.

It is generally sufficient to characterize the viscosity qualitatively since desirable product can be provided at a range of viscosities. Viscosity can significantly influence injectability. Thus, it may be desirable to select a viscosity in view of a needle or cannula size. As noted above, solid content can influence viscosity over a reasonable range. As used herein, viscosity over useful ranges can be characterized as low, medium or high, where for present purposes these are evaluated empirically through an evaluation of the pushability from the syringe during delivery.

Cohesion contributes to surface tension, which can be evaluated by a contact angle. Viscosity can also relate to the cohesion, although water has a high surface tension and a not particularly high viscosity. From a practical perspective, cohesion can be evaluated empirically in clinical studies since the tissues on which the composition is applied provides a unique environment. Generally, though, it is desirable for the flowable composite composition to remain at the location originally placed.

Storage stability, e.g., pot life, can be evaluated in vitro as a property of the flowable composite composition. Specifically, the storage stability of the flowable composite composition can be the time over which the flowable composite composition is deliverable using the prescribed delivery approach for the application, while simultaneously gelling after delivery into contact with bodily fluids in an acceptable time unless specifically indicated otherwise. In some embodiments, it may be expected that some hydrogel precursor crosslinking would occur prior to end of the storage stability time period, but significantly less crosslinking than occurs at gelation. In some embodiments, some hydrolysis can take place prior to delivery. For example, some hydrolysis of an electrophilic ester can take place during storage, and since hydrolysis removes crosslinking groups, the gel time following delivery can be observed to lengthen. Generally, the storage stability at room temperature is at least about 10 minutes, in other embodiments at least about 15 minutes, in additional embodiments from about 20 minutes to about 2 days, in some embodiments from about 30 minutes to about 24 hours, in further embodiments form about 45 minutes to about 15 hours and in other embodiments from about 1 hour to about 10 hours. Another time parameter is the gel time, which is the time over which the hydrogel precursors crosslink sufficiently such that a composite material no longer flows, for convenience, gel time can be measured in vitro. Complete crosslinking can take significantly longer, but once gelation occurs, the sealant composition is generally secured in its deposit location. The crosslinking in contact with the tissue generally provides reasonable adhesion to the tissue. Gel times are generally desired to be short. In some embodiments, the gel time is no more than 5 minutes, in further embodiments no more than 4 minutes, in additional embodiments, no more than 3 minutes, in some embodiments no more than about 2 minutes, in some embodiments no more than about 1 minute, in other embodiments from about 2 seconds to about 90 seconds, and in further embodiments from about 10 seconds to about 60 seconds, although in general, the upper cutoff can be selected for a specific application, such as 5 seconds or 10 seconds to 20 seconds, 30 seconds, 40 seconds, 50 seconds, 60 seconds, 70 seconds, 80 seconds, 90 seconds, 100 seconds, or 110 seconds. The gel time is measured in vitro by injecting a volume of the precursor solution without the granular hydrophilic polymer at room temperature into a ⅓ volume of phosphate buffered saline (PBS) at 37° C., maintaining the combined volumes at 37° C., and recording the time until a solid or a semi-solid mass is formed, wherein the volume of the precursor solution and the volume of PBS are equal. In other words, a volume of flowable composite composition without the granular component is injected (combined) with a ⅓ volume of PBS relative to flowable composite composition. PBS is standard PBS with a pH of about 7.4, which for the purpose of clarity is considered to have 1.37 millimolar (mM) NaCl, 2.7 mM KCl, 10 (8) mM Na₂HPO₄, 1.8 (2.0) mM KH₂PO₄. A person of ordinary skill in the art will recognize that additional ranges of gel times and storage stability time within the explicit ranges above are contemplated and are within the present disclosure.

In some applications, the flowable composite composition may not swell or swell only a small amount upon contact with bodily fluids, such as when essentially hydrated prior to delivery. The swelling can be considered to be the ratio of the volume of the fully crosslinked material divided by the volume of delivered material. The placement of the flowable composite composition may only provide contact with bodily fluids along a portion of the material surface and may not readily fully contact bodily fluids. With a quick gel time, the gelling only occurs where good contact with bodily fluid occurs. Nevertheless, results confirm that good hemostatic and/or other sealing is effective even if a portion of the flowable composite composition in a desired period of time. Any un-gelled composition can be carefully removed without any detrimental effects on the gelled material. Thus, in vivo gel times may be empirically determined for the particular wound or other treatment location and configuration allowed for the placement of the flowable composite composition. In relevant embodiments, the foamed, non-swelling or low swelling natures of the flowable composite composition provides for conformal filing of wounds, with low trauma from the non-swelling nature, and secure placement from the foam stability, and any non-gelling of a portion of the composition is generally inconsequential. A swelling of less than 10% can be considered non-swelling.

The sealing medical patch described in the '453 application is designed for a range of suitable applications. The medical patch may be used in some less invasive procedures with delivery through a trocar or the like. But the placement of the patch can provide challenges for some placements, and it can be also challenging to fill wounds with significant depth using the patch. The dispersions described herein are injectable through a catheter or needle. The injectable nature provides for more ready access into positions in a patient using an endoscope or other appropriate tools. In addition, the injectable nature provides for ready filling of deeper wounds with ready conformal filing of the wound. Thus, these injectable hemostatic agents provide a valuable alternative product for a health care profession facing various bleeding challenges.

The absorbent granular filler can be comprised of appropriate biocompatible materials with desirable properties. The granular filler should not be soluble in aqueous solution, otherwise the dissolved macromolecular composition has more of an effect of a thickening agent than a composite reinforcing agent. The difference in effect can be significant. The absorbent granular filler, i.e. non-dissolvable reinforcing agent, provides a cohesiveness disproportionate to its bulk when the composite is delivered to a patient. At the same time, the resulting composite provides a desired stabilization of the wound and good hemostatic effect at significant bleeding sites with the application of a modest amount of pressure. The absorbent granular filler can have a suitable particle size to allow for injection through a desired cannula (needle or catheter) size, and generally with an average diameter no more than about 100 microns (0.1 mm). In some embodiments, the precursor composite is delivered by spraying. An atomizer can be attached to the delivery tool to provide for a spray effect, which can be particularly effective for a foamed composition.

Once the hydrogel crosslinks, the composite material forms a stable unitary mass until the composition degrades or is dislodged. When crosslinked in contact with the tissue, the cured composite material generally has good adhesion to the tissue. While the hydrogel can give the composite reasonable elasticity, the granular component can provide enhanced mechanical strength.

Specific applications are discussed below. In some embodiments, the composite compositions can be used as a tissue sealant and may provide hemostasis for significant wounds. Hemostasis involves coagulation such that bleeding stops, and it can be considered the first stage of wound healing. Following hemostasis, no more blood is exiting the wound. For relevant embodiments, it can be desirable for bleeds to achieve hemostasis within about 5 minutes, in further embodiments within about three minutes and in other embodiments from about 5 seconds to about two minutes. A person of ordinary skill in the art will recognize that additional ranges of hemostasis times within the explicit ranges above are contemplated and are within the present disclosure. The extent of bleeding from an organ can be evaluated based on a Spot Grade bleeding score or other validated bleeding scale. For example, bleeding can be scored on the Adam's scale, which is described in Adams et al, Journal of Thrombosis and Thrombolysis (2009) 28:1-5 (DOI 10.1007/s11239-008-2049-3), herein incorporated by reference. Generally, hemostasis can be achieved with an Adam's bleeding score of at least 4.

As noted above, the precursor composite composition can be formulated to be non-swelling or having low swelling. For use as a sealant, such as for filling a wound, it can be desirable for the sealant filling the wound to not apply significant stresses to the wounded tissue. In contrast, a patch is applied dry and would swell significantly as it hydrates. But a patch is thin and generally applied along a surface, so swelling of the patch can extend essentially into the open space. The placement of a distorted patch into cavernous wound would exhibit the same swelling and would thus apply stresses to the wound. Since the present components are generally fully hydrated prior to delivery, swelling can be controlled to be little or none. While in principle, the in situ crosslinked hydrogel portion of the composite can be designed to be somewhat swelling or even shrinking with further hydration, this can be controlled to maintain relatively low change in swelling in a positive or negative sense.

### Forming Flowable Composite Compositions and Methods of Delivery

The flowable composite compositions comprise three components (granular and two hydrogel precursors) in a solvent along with optional additional additives. In principle, the components can be added in any order and mixed, but pragmatic reasons indicate a desirable process order to achieve a well-mixed composite composition. For the formation of foamed flowable composite compositions, sufficient force should be applied to incorporate gas bubbles, such as air bubbles, into the composition. In the exemplified embodiments, the force is applied during the processing to fragment the granular material during the mixing process to form a well mixed composition. If the granular material is preformed, the composition can be whipped appropriately or similarly processed to incorporate gas into the composite to foam the material. Once a well-mixed flowable composite composition is formed, it is generally designed for delivery through a desired delivery tool, such as a cannula, catheter, sheath, needle, dropper or the like. In some embodiments, an atomizer can be mounted on the delivery tool to spray the composition form the delivery tool. A target tissue is generally wet with blood or other suitable bodily fluids to initiate crosslinking of the hydrogel polymers and corresponding gelling of the composite. As described above, the flowable composition generally is designed to have properties allowing it to remain at the delivery location as gelling initiates, and the gelled composition generally adheres to the delivery location. Forming of a foam provides additional cohesion supporting maintenance of the composition at the deposit location. Flowable foams generally exhibit complex rheology and non-Newtonian behavior. Thus, the flowable foam can stay in place but readily flow with the application of shear. This allows for conformal filling during delivery but stable placement following injection, while allowing for some sculpting of the as deposited foam prior to gelling.

With respect to forming the flowable composite composition, it is generally desirable to have the hydrogel precursors well dissolved so that crosslinking can occur quickly and completely following delivery of the composite to tissue. By dissolving the precursors in solvent free of granular components, the state of dissolution can be observed visually, and the presence of the other species does not interfere or slow the mixing to dissolve the precursors. Once the hydrogel precursors are well dissolved, the granular material can be added to the dissolved precursor solution, or the granular material can be separately hydrated in a separate quantity of solvent followed by the mixing of the dissolved hydrogel precursors and the dispersed granular material. The mixing process to form the flowable composite composition or steps thereof may or may not be vigorous enough to further fragment the granular material. In any case, the flowable composite composition following blending should be well mixed with the hydrogel precursors well distributed and the granular material at least being initially hydrated.

While in some embodiments, especially embodiments with little or no swelling, the hydrophilic granular components can be well hydrated prior to delivery, such as by injection, in other embodiments, the granular material can be combined with the dissolved hydrogel precursors with insufficient time prior to delivery to fully hydrate the granular particles. The timing for the addition of the granular particles can be used to control the degree of hydration of the granular particles, which in turn influences the hydration as well as the absorption of bodily fluids from the application site. Generally, it is still desirable to provide a good mixture of the partially hydrated granular particles and the hydrogel precursor, which can then be delivered once the good mixture is formed. The timing of hydration and the corresponding degree of hydration can be used to adjust the interaction of the delivered precursor composite composition and resulting interaction with the wound or other site of delivery in contact with tissue. For applications directed to wound filling, it can be desirable to have little or no swelling but to provide conformal filling. The foamed, flowable composite composition with fully hydrated granular components can provide desirable properties.

Generally, the flowable composite compositions can be prepared from the components illustrated in FIG. 3: electrophilic hydrogel precursor 302, nucleophilic hydrogel precursor 304, granular material 306, and solvent 308. Optional ingredients are not depicted for simplicity, but various additives, such as visualization agents can also be used. In some embodiments, electrophilic hydrogel precursor 302 and nucleophilic hydrogel precursor 304 are provided as powders. The two precursors can be provided as a dry powder blend or as separate quantities of powder. Granular material 306 is a porous hydrophilic material and suitable compositions are described above. The Examples demonstrate the use of both crosslinked and non-crosslinked gelatin. Granular material 306 can be provided in any convenient form, including as a sheet, a collection of fragments, or as particulates, based on the anticipation of fragmenting to a desired average granular size. In some embodiments, granular material 306 can be initially provided as a sheet and then broken into fragments, shredded, and/or milled into a granular form with desired average particle size and particle size distribution. In some embodiments, granular material 306 may comprise two or more porous hydrophilic materials used together. Examples of distinct hydrophilic porous materials include, for example, materials with the same chemical composition but with different porosity, particulate size, or crosslinking and/or materials having a different chemical composition. Solvent 308 is an aqueous liquid as described above.

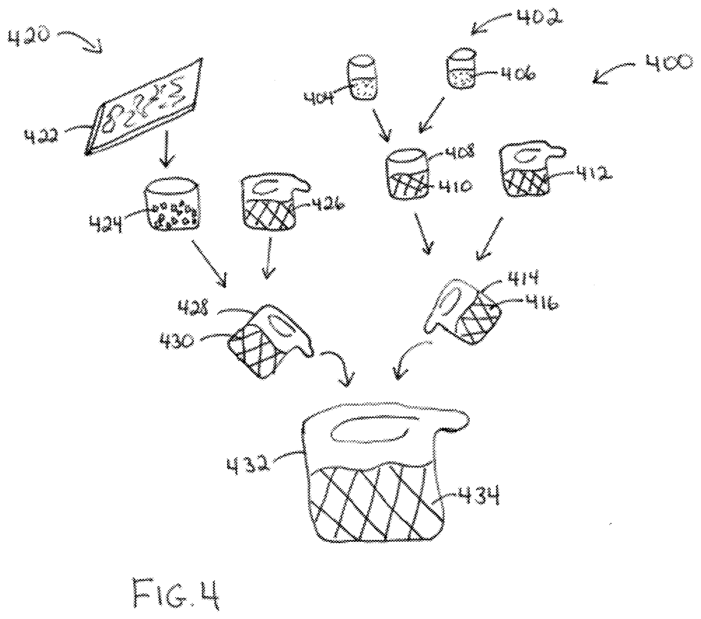

In general, at the start of a procedure or series of procedures, a granular absorbent material, an electrophilic-hydrogel precursor, and a nucleophilic-hydrogel precursor are mixed with a solvent, as noted above, and placed in a suitable dispenser/applicator. Various suitable procedures can be used for this formation of the flowable composite composition, and selection of the process can be based on convenience for the user, within the constraints of forming a well-mixed blend with appropriate properties. FIG. 4 schematically illustrates one method of making a flowable composite composition, which is the method used in the Examples.

Method 400 comprises a first sub-method step 402 in which electrophilic hydrogel precursor 404 and nucleophilic hydrogel precursor 406 are added to container 408 and mixed to form blended precursor 410. In general, electrophilic hydrogel precursor 404 and nucleophilic hydrogel precursor 406 are added to container 408 in a selected weight ratio. In some embodiments, the selected weight ratio is chosen to provide a blended precursor having an approximately equivalent number of electrophilic and nucleophilic functional groups, although other ratios can be selected, as described above. In some embodiments, electrophilic hydrogel precursor 404 and nucleophilic hydrogel precursor 406 are provided as dry powders. Blended precursor 410 may be stored or supplied as blended dry powders since the dry powders generally do not exhibit crosslinking or degradation. Suitable compositions for the precursors are described in detail above.

In the depicted embodiment, to form precursor solution 416, blended precursor 410 and solvent 412 are added to dispenser 414. In general, blended precursor 410 and solvent 412 are added to dispenser 414 in amounts to form precursor solution 416 having a selected precursor concentration. In some embodiments, dispenser 414 may be a syringe that is pre-filled with blended precursor 410, and solvent 412 may be drawn into the syringe with a needle or catheter to form precursor solution 416. In further embodiments, a syringe barrel may have a volumetric capacity greater than the volume of the precursor solution to provide for sufficient head space for mixing the precursor solution via shaking, swirling, tilting, or the like of the syringe barrel. While manual mixing can be sufficient, vortexing or other mechanical mixing assists can be used if desired to facilitate the dissolving process. In some embodiments, the volumetric capacity of the syringe barrel may be from about 1.25 to about 2.5 times the volume of solvent 412, in further embodiments from about 1.5 to about 2.25 times the volume of the solvent. A person of ordinary skill in the art will recognize that additional ranges of relative volumes within the explicit volumes above are contemplated and are within the present disclosure. In some embodiments, the two precursors can be initially provided as a dry powder blend, such that forming a precursor solution involves addition of solvent 412 without a separate precursor blending step. In other embodiments, forming a precursor solution involves the addition of electrophilic hydrogel precursor 404, nucleophilic hydrogel precursor 406, and solvent 412 to dispenser 414, without a separate precursor blending step. In other embodiments, electrophilic hydrogel precursor 404 is combined with a portion of solvent 412 to form an electrophilic hydrogel precursor solution and nucleophilic hydrogel precursor 406 is combined with a portion of solvent 412 to form a nucleophilic hydrogel precursor solution, such that formation of precursor solution 416 involves the addition of the electrophilic hydrogel precursor solution and the nucleophilic hydrogel precursor solution to dispenser 414. Precursor solution 416 can be mixed until electrophilic hydrogel precursor 404 and nucleophilic hydrogel precursor 406 are dissolved. In some embodiments, dissolution can be determined by the lack of visible particles to the naked eye. Generally, suitable mixing can be performed with shaking by hand, but an effective approach would involve passing back and forth between two connected syringes. In some embodiments, mixing can be performed with a vortex mixer or other mechanical mixing approach. In some embodiments, mixing is performed with the addition of heat. A convenient approach for use by medical professionals involves transfers back and forth between two syringes connected tip to tip using luer-to-luer connectors, which can also result in entrapment of air to provide foam formation if one or both syringes are prepared with a volume of air to be blended into the precursor solution. In some embodiments, dissolving occurs in less than about 15 minutes, in some embodiments no more than about 10 minutes, and in other embodiments from about 20 seconds to about 8 minutes. In any case, mixing of the precursors is performed until the precursors are visibly dissolved. A person of ordinary skill in the art will recognize that additional ranges of dissolving times within the explicit ranges above are contemplated and are within the present disclosure. If electrophilic hydrogel precursor 404 and/or nucleophilic hydrogel precursor 406 are solid at room temperature, consistent low dissolving times may be obtained by adding the precursor(s) to the dispenser as a powder, such as a fine, flowable powder since high surface area facilitates dissolving. Dispenser 414 may be a vial, a syringe, or any other suitable container or device. In some embodiments, precursor solution 416 can be drawn into a subsequent dispenser, such as a syringe, and optionally sealed, such as with a syringe cap.

In the exemplified process, further referring to FIG. 4, method 400 comprises a second sub-method step 420 in which hydrophilic filler sheet 422 is processed into granular material 424 for the flowable composite. As exemplified, processing into granules involves breaking hydrophilic filler sheet 422 into fragments, followed by shredding, and/or milling into granules of desired particle properties, such as size, size distribution and shape. As exemplified, fragments of broken up sheet and solvent 426 are added to dispenser 428 to form hydrophilic material suspension 430. Granular material 424 may correspond to granular material 306 as described above with respect to FIG. 3, although completion of the formation of the granular material may be subsequently completed and may never be present in separate form prior to processing. Granular material 424 and solvent 426 can be added to dispenser 428 in an amount to provide a selected concentration of the absorbent filler. In some embodiments, as exemplified in Example 1, the amount of granular material 424 is selected by first selecting an area of hydrophilic filler sheet 422 to use. For example, a 16 cm² area of hydrophilic filler sheet 422 could be provided 2 cm by 8 cm section of the sheet. Based on the density of the sheet, this area of sheet can be converted to a weight to obtain a corresponding weight percent. In some embodiments, the granular material can be initially provided in granular form with a desired weight and either with the desired particle size or a size that can be readily achieved through the processing steps. In such embodiments, forming a granular material dispersion involves addition of solvent 426 without a separate hydrophilic material sheet processing step. In some embodiments, dispenser 428 may be a syringe that is pre-filled with granular material 424, and solvent 426 may be drawn into the syringe with a needle or catheter to form hydrophilic material suspension 430. In further embodiments, a syringe barrel may have a volumetric capacity greater than the volume of the composite filler solution to provide for sufficient head space for mixing the composite filler solution via shaking, swirling, tilting, or the like of the syringe barrel. As described further below, the inclusion of air or other gas with appropriate manipulations to entrap the air or gas into the composition can provide a foamed compositions. In some embodiments, the volumetric capacity of the syringe barrel may be from about 1.25 to about 2.5 times the volume of solvent 426, in further embodiments from about 1.5 to about 2.25 times the volume of the solvent. A person of ordinary skill in the art will recognize that additional ranges of relative volumes within the explicit volumes above are contemplated and are within the present disclosure. The surface tension of the liquid provides a boundary with respect to the amount of gas that can be introduced while forming a consistent foam. While lower amounts of gas may be desirable to achieve a foam with desired properties, generally it is desirable to use an amount of gas that does not result in an inhomogeneous material lacking incorporation of all of the gas into a foam. In some embodiments, hydrophilic material suspension 430 can be gently mixed to promote hydration. In some embodiments, hydration can be determined by the lack of visible particles to the naked eye. In general, hydration occurs in less than about 15 minutes, and full equilibrium hydration can be evaluated for particular granules. In some embodiments, if partial hydration, such as 75% hydration is desired, the hydration time can be reduced accordingly. A person of ordinary skill in the art will recognize that additional ranges of hydration times within the explicit ranges above are contemplated and are within the present disclosure. Dispenser 428 may be a vial, a syringe, or any other suitable container or device. In some embodiments, hydrophilic material suspension 430 can be drawn into a subsequent dispenser, such as a syringe, and optionally sealed, such as with a syringe cap.

Further referring to FIG. 4, precursor solution 416 and hydrophilic material suspension 430 are combined in dispenser 432 to form flowable composite composition 434. In general, flowable composite composition 434 are mixed using any convenient method. The mixing process can also be used to foam the flowable precursor composition. Some entrapped air can be introduced with the ingredients to be mixed. Also, a selected volume of air or other inert gas can be introduced into the mixing structure for entrapping in the resulting foamed material. For example, if syringes are used for the mixing, a volume of gas/air can be placed into the syringe for mixing. For the foaming of a single dose of material, the volume of introduced gas/air can be from about 0.1 mL to about 4 mL, in further embodiments form about 0.25 mL to about 3 mL and in other embodiments form about 0.4 mL to about 2.5 mL. These volume ranges for entrapped air are based on a reasonable sample size for the precursor solution. More generally, a precursor solution for deposition can be from about 5 vol % to about 85 vol % entrapped gas, in further embodiments from about 10 vol % to about 82.5 vol %, in some embodiments from about 15 vol % to about 80 vol % and in other embodiments from about 20 vol % to about 77.5 vol % entrapped gas relative to the total volume of the precursor sample. A person of ordinary skill in the art will recognize that additional ranges of gas/air volumes or relative volumes within the explicit ranges above are contemplated and are within the present disclosure. Selection of a desired mixing methods may be influenced by whether or not further fragmenting of the granular component is desired as well as user convenience. Dispenser 432 may be a vial, a syringe, a beaker, or any other suitable container or device. In some embodiments, flowable composite composition 434 is mixed by transferring flowable composite composition 434 between dispenser 432 and a secondary dispenser one or more times. In some embodiments, mixing can involve at least 2, at least 10, at least 20, from about 2 to about 200 transfers, from about 5 to about 100 transfers, or from about 10 to about 80 transfers. In alternative embodiments, the components can be mixed, placed in a container with some head space, and vortexed for good mixing. Generally, any reasonable mixing method can be used to form a good mixture. In some embodiments, dispenser 432 can be dispenser 414 (from first sub-method 402), such that flowable composite composition 434 is formed by adding hydrophilic material suspension 430 to precursor solution 416 in dispenser 414/432. In some embodiments, dispenser 432 can be dispenser 428 (from second sub-method step 420), such that flowable composite composition 434 is formed by adding precursor solution 416 to hydrophilic material suspension 430 in dispenser 428/432. In some embodiments, dispenser 414/432 is a syringe and flowable composite composition 434 is mixed by transferring flowable composite composition 434 between dispenser 414/432 and a secondary syringe one or more times. In some embodiments, this secondary syringe can be dispenser 428. In some embodiments, dispenser 428/432 is a syringe and flowable composite composition 434 is mixed by transferring flowable composite composition 434 between dispenser 428/432 and a secondary syringe one or more times. In some embodiments, this secondary syringe can be dispenser 414. Example 1 exemplifies connecting a syringe containing a precursor solution to a syringe containing an absorbent filler solution using a Luer syringe connector and mixing the contents, via cycling the contents between the first and second syringe, to form the flowable composite composition. Such an exchange of flowable material can further fragment absorbent granular material. In some embodiments, hydrophilic filler sheet 422 and/or granular material 424 can be added directly to precursor solution 416 in dispenser 414 to form flowable composite composition 434, without the steps of forming and adding a separate hydrophilic filler solution. In some embodiments, the flowable composite composition can be further processed prior to delivery to a treatment site. In some embodiments, the flowable composite composition is directed through one or more narrow orifice in order to further homogenize the composition. In some embodiments, the narrow orifice(s) can be a needle or a series of needles. In some embodiments, a series of needles of decreasing inner diameter (i.e. increasing gauge) can be used. In some embodiments, a needle or needles can have a gauge of at least 16, 18, 20, or 21 or from about 16 to about 25, from about 18 to about 25, from about 20 to about 25, or from about 21 to about 23. While not wanting to be limited by theory, it is believed that in the exemplified embodiment this further processing breaks up the absorbent filler into smaller particles. This further processing has been demonstrated to provide a lower force for delivery of the flowable composite composition.

Once the precursors are mixed into a single solution, such as in first sub-method step 402, the time can be noted to provide a window for target delivery of the flowable composite composition prior to the composition batch expiring. Generally, batch expiration can be equated with pot life, but other pragmatic or regulatory constraints could influence batch timing. The time window may be related to the storage stability of the mixed precursors in solution with some of the time window needing to be used to complete further steps to prepare the flowable composite composition for delivery. Practical considerations can dictate setting the practical time window somewhat shorter than the sum of the storage stability and anticipated time for preparing the flowable sealant composition for delivery to provide an extra margin of safety. During this practical time window, one or multiple sealant delivery procedures can take place.

Various medical procedures can benefit from the use of the flowable composite compositions described herein. Generally, the flowable composite compositions are particularly useful for delivery into locations where the composite composition provides desirable properties with respect to maintenance of the material at the delivery site as well as providing desirable properties of the cured composition. The flowable composite compositions are also particularly useful for delivery into remote locations of the body, such as deep punctures, larger areas or spatially extended wounds, or locations accessed via laparoscopic procedures. The composites can also be used for applications with long lasting materials that can provide stable support functions. Specific applications discussed below include delivery to a bleeding site to provide hemostasis and delivery between tissue planes for prevention of formation of hematomas and/or seromas.

For laparoscopic applications, a syringe can be attached to an appropriate delivery tool, such as a cannula or a trocar, as described below and exemplified in Part B of Example 2. For open surgical or topical applications, a syringe can be used with or without an additional delivery attachment. For either embodiment, an atomizer can be attached to the end of the delivery device, such as with a luer connection. Delivery of the flowable composite composition to bleeding sites during an open surgical procedure are exemplified in Part C of Example 2. Specific applications may incorporate drug delivery. In some embodiments, therapeutic agents may include anesthetics, antibiotics, steroids, non-steroidal anti-inflammatory drugs (NSAID), chemotherapeutics, or a combination thereof.

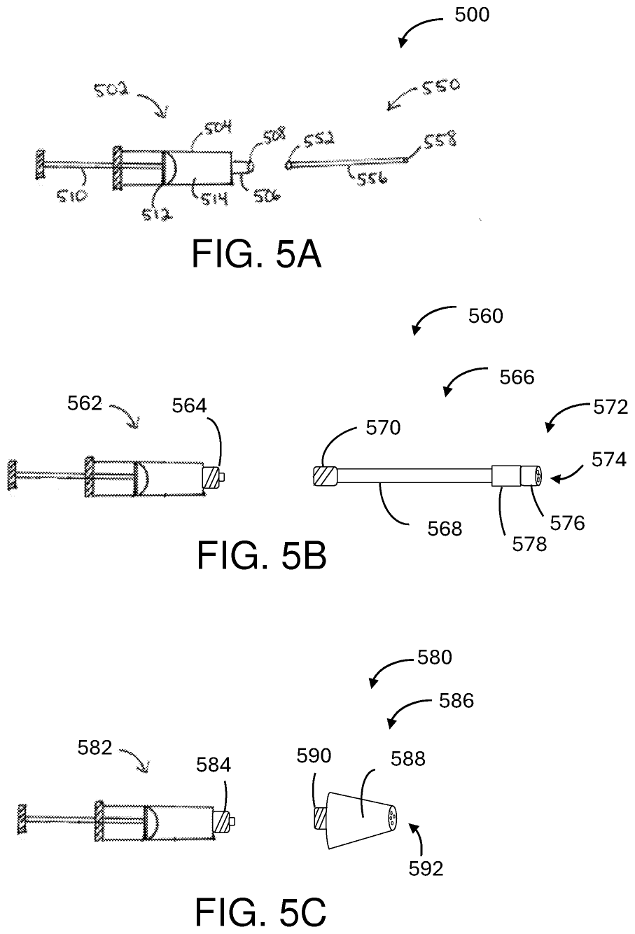

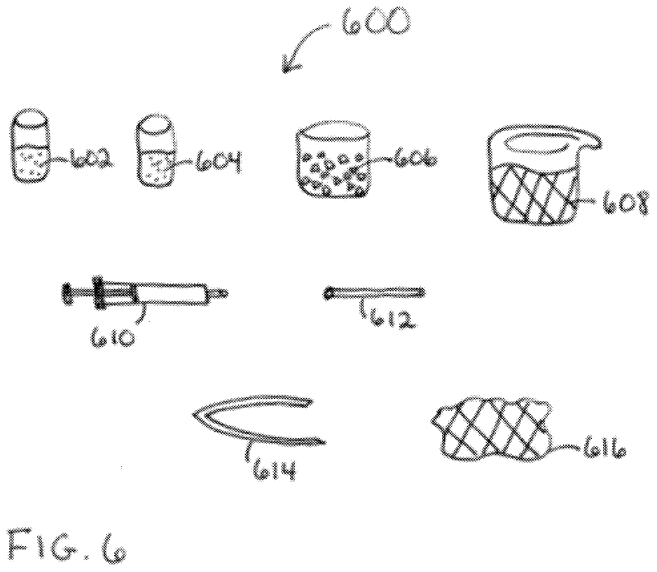

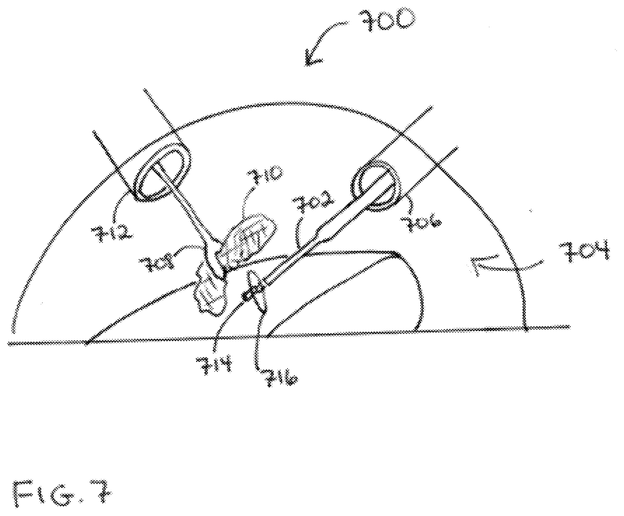

FIG. 5A displays a dispenser system suitable for delivering the flowable composite composition described herein. Dispenser 502 is shown as part of dispenser system 500 which also includes optional delivery attachment 550. Dispenser 502 has syringe 504 having connector 506, such as a standard luer lock fitting. Connector 506 connects to distal outlet 508. Dispenser 502 also has a plunger 510 which is attached to a plunger seal 512. Plunger seal 512 contacts composition 514 within syringe 504. In some embodiments, optional delivery attachment 550 is connected to dispenser 502 at proximal end 552 via connector 506. Optional delivery attachment 550 has conduit 556 which has delivery tip 558 at its distal end. Delivery of composition 514 through distal outlet 508 or delivery tip 558 may be controlled by pressing plunger 510. In some embodiments, conduit 556 may be a catheter or a trocar or another type of conduit having an open-ended distal port. Dispensers for use with the flowable composite composition are not particularly limited and include commercially available applicators.

The dispenser system of FIG. 5A can be effectively used for a range of applications. Conduit 556 can be a generic catheter or trocar or can be specifically engineered to facilitate a particular procedure. For delivery into an open surgical site or onto a topical site, it may be convenient to use dispenser 502 without optional delivery attachment 550 or with an alternative attachment. The applicator or dispenser system, for the flowable composite composition can have alternative designs that may be more suited to a particular application whether or not the dispenser system of FIG. 5A can be used.

The dispenser system can be configured to deliver the flowable composite composition as a foam. FIG. 5B is an illustration of dispenser system 560 comprising dispenser 562 and optional delivery attachment 566. Dispenser 562 comprises a dispenser such as the syringe described for FIG. 5A with male luer lock fitting 564 in place of connector 506 and distal outlet 508. Optional delivery attachment 566 comprises conduit 568 with female luer lock fitting 570 at one end of the conduit for mating with male luer lock fitting 564 to provide a secure, leak resistant connection. At the other end of conduit 568 is atomizer 572 comprising atomization element 576 connected to the conduit by connector 578. Connector 578 may comprise any suitable type of connector such as a mating portion of a luer slip or luer lock connector that mates with a connector (not shown, but equivalent to 564) of conduit 568. Atomization element 576 can comprise an orifice 574 through which a mixture of the flowable composite composition and air are ejected as the plunger of dispenser 562 is pressed. In general, various atomizer structures are known, but commercial atomizers with luer connectors are known in the art and commercially available based on a constricted orifice to provide the spray. In general, the orifice can have a diameter from about 50 microns to about 800 microns, in further embodiments form about 100 microns to about 600 microns, and in other embodiments from about 150 microns to about 500 microns. A person of ordinary skill in the art will recognize that additional ranges of orifice diameters within the explicit ranges above are contemplated and are within the present disclosure.

Another example of a dispenser system that can be used to deliver the flowable composite composition as a foam is shown in FIG. 5C. Dispenser system 580 comprises dispenser 582 and optional delivery attachment 586. Dispenser 562 comprises a dispenser such as the syringe described for FIG. 5A with male luer lock fitting 584 in place of connector 506 and distal outlet 508. Optional delivery attachment 586 comprises body 588 configured with female luer lock fitting 590 at one end for mating with male luer lock fitting 584 to provide a secure, leak resistant connection. Inside the body comprises a distal orifice 592 through which a mixture of the flowable composite composition and air are ejected as the plunger of dispenser 562 is pressed.

FIG. 6 illustrates one embodiment of a kit for making and delivering the flowable composite composition to a tissue. Kit 600 includes electrophilic hydrogel precursor 602, nucleophilic hydrogel precursor 604, granular hydrophilic filler 606, solvent 608, dispenser 610, optional delivery attachment 612, optional grasper 614, and optional compression device 616. In some embodiments, electrophilic hydrogel precursor 602 and nucleophilic hydrogel precursor 604 can be provided as separate dry powders. In other embodiments, electrophilic hydrogel precursor 602 and nucleophilic hydrogel precursor 604 can be provided as a blended dry powder. Granular hydrophilic filler 606 is shown in FIG. 6 in particulate form, but in some embodiments the hydrophilic filler can be provided in sheet or other bulk form or other convenient form for formation of granular components at the time of use. In general, electrophilic hydrogel precursor 602, nucleophilic hydrogel precursor 604, granular hydrophilic filler 606, and/or solvent 608 can have features as described above for electrophilic hydrogel precursor 302, nucleophilic hydrogel precursor 304, granular material 306, and solvent 308. In some embodiments, dispenser 610 and/or optional delivery attachment 612 can have features as described above for dispenser system 500. In some embodiments, optional grasper 614 can be tweezers or forceps or any other suitable device for grasping compression device 616. In general, compression device 616 can be a sterile material which can be used to apply pressure to the flowable composite composition after delivery. In some embodiments, compression device 616 is an absorbent material, such as gauze or the like. In some embodiments, compression device 616 is wet gauze.

For hydrated flowable composites, the gelling can be non-uniform since the portion contacting bodily fluids gels quickly and any portions more distant from bodily fluids may gel more slowly. Nevertheless, hemostasis can be achieved quickly for even significant bleeds. But the application of pressure in these circumstances generally is not needed and can be problematic. Since the composite surface may not gel quickly, it may absorb into a compress and subsequent removal of the compress may then damage a seal formed by the cured composite material. With foamed flowable composite, a compression device may not be needed for most treatment sites to maintain the composition on target. After hemostasis or an otherwise appropriate seal is formed, any un-gelled composition at the top of the composition composite can be wiped away to provide for ending the procedure while mitigating any random migration of uncured material.

### Therapeutic Agents

Composite hydrogel compositions may be used advantageously for localized or systemic therapy with a therapeutic agent, such as a drug. Biologically active agents or drug compounds that may be added and delivered from the cured composite compositions include: proteins, glycosaminoglycans, carbohydrates, nucleic acid, inorganic and organic biologically active compounds where specific biologically active agents include but are not limited to: enzymes, anti-infectives, antifungals, anti-inflammatoires, antineoplastic agents, local anesthetics, hormones, angiogenic agents, anti-angiogenic agents, growth factors, antibodies, a pain reliever, an anesthetic, a steroid, neurotransmitters, psychoactive drugs, anticancer drugs, chemotherapeutic drugs, anti-tumor drug, a glaucoma drug, drugs affecting reproductivity, genes, oligonucleotides a glycosaminoglycan, an antibiotic, a non-steroidal anti-inflammatory drug, an antineoplastic agent, an intraocular pressure-lowering agent, a neurotransmitter, a psychoactive drug, an anticancer drug, cells, a cellular product, or combinations thereof. As used herein, therapeutic agent refers to any biologically active agent or drug.

In the context of previous uses of hydrogels for medical applications, various formulations have been developed to provide for effective therapeutic agent delivery from hydrogels with selective release rates. In general, to prepare a crosslinked hydrogel composition, the therapeutic agent can be mixed with the crosslinkable polymer precursors prior to, at the time of or following making an aqueous precursor solution. The flowable composite compositions described herein can be similarly prepared. A mixture of hydrogel precursors, granular hydrophilic polymer and therapeutic agent then can be injected as a flowable composite composition for in situ polymerization at the target delivery site. The biodegradation rate of the polymer may or may not be selected to coordinate with the therapeutic agent elution rate depending on the nature of the therapeutic agent and the hydrogel.

In some embodiments, a therapeutic agent or agents are present in a separate phase when crosslinkable polymers are reacted to produce a crosslinked polymer network or gel. This phase separation can prevent participation of therapeutic agent in the chemical crosslinking reaction, such as reaction between NHS ester and amine group. The separate phase also helps to modulate the release kinetics of active agent from the crosslinked material or gel, where ‘separate phase’ could be oil (oil-in water emulsion), biodegradable vehicle, and the like. Biodegradable vehicles in which the active agent may be present include: encapsulation vehicles, such as microparticles, microspheres, microbeads, micropellets, and the like, where the active agent is encapsulated in a bioerodable or biodegradable polymers such as polymers and copolymers of: poly(anhydride), poly(hydroxy acid) s, poly(lactone) s, poly(trimethylene carbonate), poly(glycolic acid), poly(lactic acid), poly(glycolic acid)-co-poly(glycolic acid), poly(orthocarbonate), poly(caprolactone), crosslinked biodegradable hydrogel networks like fibrin glue or fibrin sealant, caging and entrapping molecules, like cyclodextrin, molecular sieves and the like. Microspheres made from polymers and copolymers of poly(lactone) s and poly(hydroxy acid) are of particular interest as biodegradable encapsulation vehicles.

In general, the therapeutic agent or encapsulated therapeutic agent may be present in solution or suspended form for incorporation into the flowable composite composition. The chemical reaction between the nucleophile and the electrophile readily takes place to form a crosslinked composite composition suitable for release of the active agent to the host. Such methods of therapeutic agent delivery may find use in systemic and/or local administration of the therapeutic agent, depending on the degree of vascularization at the delivery site.

In using the crosslinked composition for therapeutic agent delivery as mentioned above, the amount of the dosage agent introduced in the host appropriately can depend upon the particular therapeutic agent and the condition to be treated, as well as the practical loading level into the product cured composite material. Once the cured composite material is formed, release of a therapeutic agent may ensue resulting in localized treatment to the area. In some locations, blood flow can result in systemic distribution of the therapeutic agent, or perhaps organ-wide distribution of the therapeutic agent.

Certain embodiments of the therapeutic agent delivery are accomplished by providing compositions and methods to control the release of relatively low molecular weight therapeutic species using hydrogels within the composites described herein. In accordance with the principles herein, a therapeutic species first can be dispersed or dissolved within one or more relatively hydrophobic rate modifying agents to form a mixture. The mixture may be formed into microparticles, which are then entrapped within a bioabsorbable hydrogel matrix along with the granular hydrophilic polymer so as to release the water soluble therapeutic agents in a controlled fashion. Thus, microparticles comprising a therapeutic agent can be also blended appropriately into the flowing composite material as a well blended component to be entrapped by the cured composite material.

In one method of forming therapeutic agent delivery hydrogels, hydrogel microspheres are formed from polymerizable macromers or monomers by dispersion of a polymerizable phase in a second immiscible phase, wherein the polymerizable phase contains at least one component required to initiate polymerization that leads to crosslinking and the immiscible bulk phase contains another component required to initiate crosslinking, along with a phase transfer agent. Pre-formed microparticles containing the water-soluble therapeutic agent may be dispersed in a hydrogel precursor solution as described above.

Embodiments of the compositions and methods for forming composite hydrogel-based matrices and microspheres can have entrapped therapeutic compounds. In one embodiment, a bioactive agent is entrapped in microparticles having a hydrophobic nature (herein called “hydrophobic microdomains”), to retard leakage of the entrapped agent. In some embodiments, the composite materials that have two phase dispersions, where both phases are absorbable, but are not miscible. For example, the continuous phase may be a hydrophilic network (such as a hydrogel, which may or may not be crosslinked) while the dispersed phase may be hydrophobic (such as an oil, fat, fatty acid, wax, fluorocarbon, or other synthetic or natural water immiscible phase, generically referred to herein as an “oil” or “hydrophobic” phase).

If an oil phase is used, the oil phase entraps the drug and provides a barrier to release by slow partitioning of the drug into the hydrogel. The hydrogel phase in turn protects the oil from digestion by enzymes, such as lipases, and from dissolution by naturally occurring lipids and surfactants. The latter are expected to have only limited penetration into the hydrogel, for example, due to hydrophobicity, molecular weight, conformation, diffusion resistance, etc. In the case of a hydrophobic drug which has limited solubility in the hydrogel matrix, the particulate form of the drug may also serve as the release rate modifying agent.

Hydrophobic microdomains, by themselves, may be degraded or quickly cleared when administered in vivo, making it difficult to achieve prolonged release directly using microdroplets or microparticles containing the entrapped agent in vivo. In accordance with some embodiment, however, the hydrophobic microdomains are sequestered in a gel matrix. The gel matrix protects the hydrophobic microdomains from rapid clearance, but does not impair the ability of the microdroplets or microparticles to release their contents slowly. Visualization agents may be included, for instance, in the gel matrix or the microdomains.

In other aspects of the compositions, the active agent is hydrophobic and poorly water soluble, and itself a hydrophobic domain. The insoluble drug particle may be micronized in nature to allow for homogenous suspension throughout the entirety of the hydrogel and to allow for consistent application without clogging of the delivery system. On formation of the in situ system, the hydrophobic poorly soluble drug particles remain confined within the gel matrix, forming a concentrated environment that acts as a diffusion barrier to further drug dissolution as the drug is required to dissolve and then diffuse out of the matrix for release. Poorly soluble hydrophobic drugs may undergo zero or first order release kinetics as a function of their solubility, the amount loaded into the gel, and the surface area of the hydrogel as applied. In more confined environments such as ocular or subdermal, release kinetics may be further extended as local tissue is saturated with drug, further restricting the diffusion path out of the hydrogel.

In another aspect of the compositions, the hydrogel microspheres are formed having a size that provide selective deposition of the microspheres, or may linked with ligands that target specific regions or otherwise affect deposition of the microspheres within a patient's body.

Controlled rates of drug delivery also may be obtained with the system of the present invention by degradable, covalent attachment of the bioactive molecules to the crosslinked hydrogel network. The nature of the covalent attachment can be controlled to enable control of the release rate from hours to weeks or longer. By using a composite made from linkages with a range of hydrolysis times, a controlled release profile may be extended for longer durations.

In certain embodiments, the addition of the therapeutic agent does not elevate the existing pH of the essentially unbuffered system such that it overwhelms the acid conjugate and similarly does not add significant buffer capacity. In other embodiments, encapsulation or phase separation of the therapeutic agent can be effective to isolate the therapeutic agent to avoid significantly altering the pH or buffer capacity of the flowable composite composition. The choice of therapeutic agent is generally influenced by the particular medical applications. In that context, some specific drugs/therapeutic agents are mentioned in the following discussion of specific medical indications.

### Laparoscopic Procedures

The injectable nature of the flowable composite composition makes it well suited for laparoscopic procedures in which instruments as well as compositions are delivered through a cannula or trocar. In some embodiments, the flowable composite compositions are injectable through relatively thin needles, so that percutaneous delivery correspondingly follows. Since crosslinking does not proceed at significant rate until contact with physiological fluids, clogging of the catheter/needle should not occur. The specific selection of delivery tools can be based on the specifics of the procedure. Instruments for laparoscopic procedures are readily available commercially.

Volumes delivered generally can be adjusted for a particular procedures. In general, the precursor composite compositions can have a volume for delivery from about 1 mL to about 50 mL, in further embodiments, from about 2 mL to about 40 mL, and in other embodiments from about 5 mL to about 30 mL. In use, a medical professional can adjust the delivered amount accordingly, such as applying a selected fraction of the prepared amount. Similarly, multiple units of a commercial prepared amount can be used as needed. Commercial units may be supplied in different sizes. A person of ordinary skill in the art will recognize that additional ranges of volumes within the explicit ranges above are contemplated and are within the present disclosure.

For laparoscopic procedures, the cured hydrogel composite compositions are generally resorbable in a reasonable period of time so that they clear naturally from the patent through forming non-toxic soluble breakdown products. As noted above, the degradation of the cured material can take place by hydrolysis, enzymatic action, combination thereof or the like. The hydrogel component and the granular components may degrade at similar or different rates, and each material can be engineered to have degradation within a target window, although in vivo degradation time have a spread based on specific patient and use. If desired, the composite compositions can be engineered for long lasting or essentially permanent placement in the patient. As noted above, long lasting compositions can be removed by generally acceptable chemical methods if desired.

FIG. 7 is an illustration of delivery of a flowable composite composition for use in a laparoscopic procedure 700. Trocar 702 has been introduced into laparoscopic site 704 via cannula 706. In some embodiments, forceps 708 and gauze 710 can be introduced into laparoscopic site 704 via cannula 712. Forceps 708 holds gauze (or similar appropriate material) 710. For deep wounds, the flowable composite composition can displace blood in the wound and then conformally fill the wound, and then the sealant can set. In appropriate embodiments, the gauze can help to stabilize the sealant as it cures, or gauze 710 can be used to wipe away any uncured flowable composite composition once sealing and/or hemostasis is achieved. In contrast, for a deep wound, especially an irregular wound, a patch may have difficulties forming a good seal as blood and/or other fluids escape the wound and pushing away the patch edge unless significant pressure is applied. Trocar 702 is shown delivering flowable composite composition 714 to treatment site 716. In some embodiments, flowable composite composition 714 can be delivered to cover and/or extend beyond treatment site 716. In some embodiments, trocar 702 can be inserted at least partially into treatment site 716 to deliver flowable composite composition 714 below the surface of the organ being treated. In some embodiments, flowable composite composition 714 can be initially delivered to selected portions of treatment site 716. After the selected delivery protocol, if useful, forceps 708 can help guide gauze 710 to provide pressure to the delivered flowable composite composition. In some embodiments, treatment site 716 can be a wound site or a surgical site ready for closure. In some embodiments, treatment site 716 is a puncture, a gouge, a tear, an incision, or a surface abrasion. In some embodiments, treatment site 716 is a surface between tissue planes. In some embodiments, treatment site 716 is a surface that, upon delivery of flowable composite composition 714 and closure of the treatment site, will be separated from another tissue plane by the delivered composition. In some embodiments, flowable composite composition 714 can be used in laparoscopic procedure 700 to provide hemostasis. In some embodiments, flowable composite composition 714 can be used in laparoscopic procedure 700 to provide separation between adjacent tissue planes.

### Open, Dermal and Subdermal Procedures

In addition to laparoscopic placement, the flowable composite composition can be used in open procedures for sealing wounds in organs or other placement, as an alternative to use of a medical patch. The flowable composite composition can be particularly desirable for larger wounds, deeper wounds or wounds with less well defined boundaries. Similarly, the flowable composite compositions can be used for sealing wounds on the skin, dermal wounds, or for assisting the sealing of surgical sites, which can involve adherence of large seams of tissue being rejoined. In particular, the wound can be a defect associated with a suture line, a puncture wound, a bullet wound, a cavity, a perforation, a gouge, a biopsy punch hole, a graft interface, an abrasion wound, a tissue harvest site, or a combination thereof. An example is described relating to an abdominoplasty procedure, although the composition can be used as a sealant for any large abdominal incisions. With temporary or more permanent formulations, the flowable composite composition can be suitable for subdermal injection as a tissue filler, for cosmetic or therapeutic use.

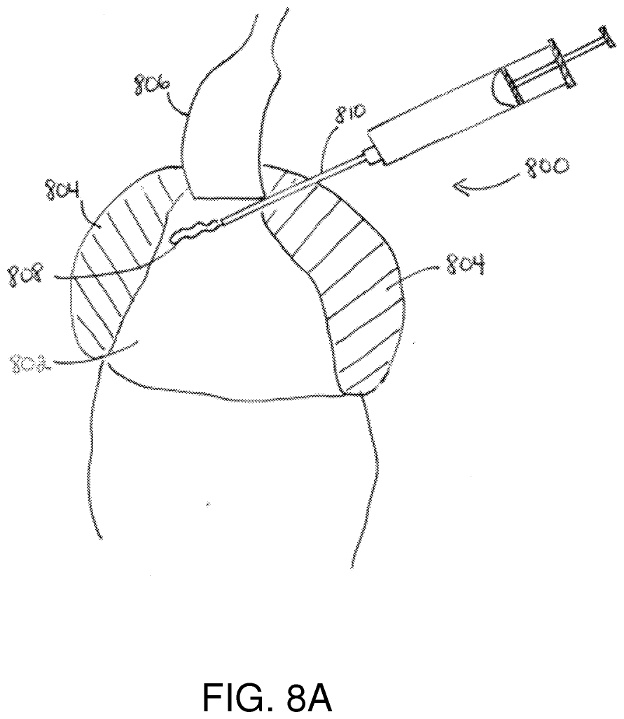

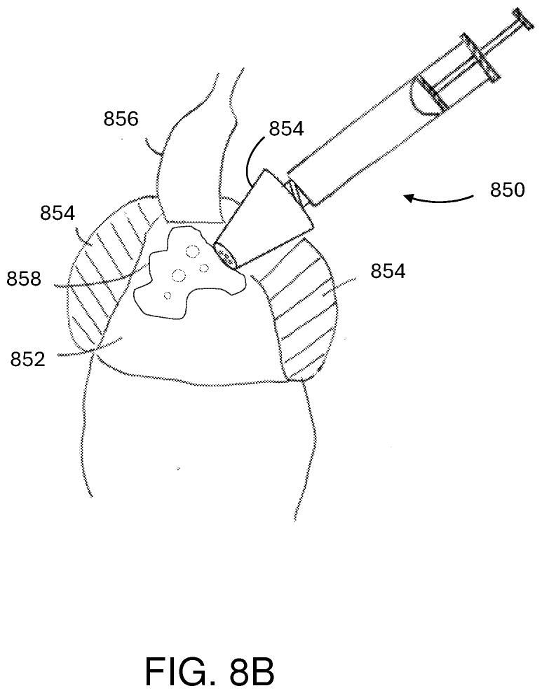

FIG. 8A is an illustration of delivery of a flowable composite composition for use in abdominoplasty procedure 800. Tissue layer 802 has been exposed via displacing tissue layer 804 with the use of retractor 806. Flowable composite composition 808 has been introduced onto tissue layer 802 via trocar 810. In some embodiments, flowable composite composition 808 can be delivered to cover tissue layer 802. In some embodiments, flowable composite composition 808 can be delivered to selected portions of tissue layer 802. After the selected delivery protocol, pressure may be applied to the delivered flowable composite composition, such as with the use of gauze or other appropriate material, and then tissue layer 804 can repositioned onto tissue layer 802. In some embodiments, after the selected delivery protocol, tissue layer 804 is repositioned onto tissue layer 802 which provides pressure to the delivered flowable composite composition through its weight. In some embodiments, additional manual pressure may be applied to the delivered flowable composite composition though tissue layer 802. In some embodiments, tissue layer 804 can repositioned onto tissue layer 802 prior to the delivery of flowable composite composition 808, and flowable composite composition 808 can be delivered between the adjacent tissue layers. Additional manual pressure, beyond the pressure provided by the weight of tissue layer 802, may be applied to facilitate desirable sealing by the delivered flowable composite composition upon curing.

FIG. 8B is an illustration of delivery of a flowable composite composition for use in abdominoplasty procedure 850. Tissue layer 852 has been exposed via displacing tissue layer 854 with the use of retractor 856. Foamed flowable composite composition 858 has been introduced onto tissue layer 852 via atomizer 860. In some embodiments, foamed flowable composite composition 858 can be delivered to cover tissue layer 852. In some embodiments, flowable composite composition 858 can be delivered to selected portions of tissue layer 852. After the selected delivery protocol, pressure may be applied to the delivered flowable composite composition, such as with the use of gauze or other appropriate material, and then tissue layer 854 can repositioned onto tissue layer 852. In some embodiments, after the selected delivery protocol, tissue layer 854 is repositioned onto tissue layer 852 which provides pressure to the delivered flowable composite composition through its weight. In some embodiments, additional manual pressure may be applied to the delivered flowable composite composition though tissue layer 852. In some embodiments, tissue layer 854 can repositioned onto tissue layer 852 prior to the delivery of flowable composite composition 858, and flowable composite composition 858 can be delivered between the adjacent tissue layers. Additional manual pressure, beyond the pressure provided by the weight of tissue layer 852, may be applied to facilitate desirable sealing by the delivered flowable composite composition upon curing.

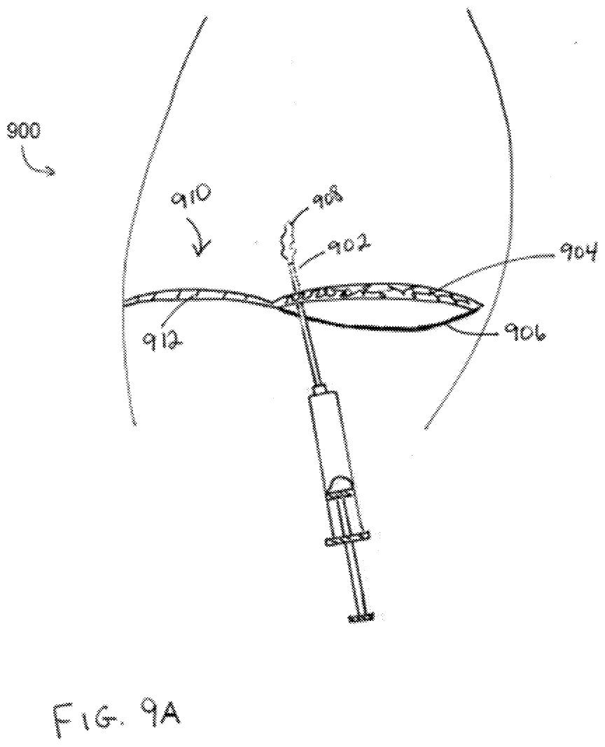

FIG. 9A is an illustration of a subcutaneous method of delivery of a flowable composite composition. The method is shown for illustration purposes for use in abdominoplasty procedure. In method 900, trocar 902 has been inserted between tissue layer 904 and tissue layer 906. Flowable composite composition 908 has been delivered between tissue layer 904 and tissue layer 906 via trocar 902. In some embodiments, flowable composite composition 908 can be delivered to fill a surgical space between tissue layer 904 and tissue layer 906. In some embodiments, flowable composite composition 908 can be delivered in a series of discrete steps, such as after the closure of a surgical region 910 with sutures 912. In some embodiments, flowable composite composition 808 can be delivered between tissue layer 904 and tissue layer 906 in a pre-sutured surgical region, such as surgical region 910. In some embodiments, after the selected delivery protocol, tissue layer 904 is repositioned onto tissue layer 906 which provides pressure to the delivered flowable composite composition through its weight. In some embodiments, additional manual pressure may be applied to the delivered flowable composite composition though tissue layer 904.

In some embodiments, the flowable composite compositions described herein can be loaded with therapeutic agents, such as antibiotics, and/or visualization agents and used for the treatment of fistulas. Fistulas are abnormal anatomical tubular structures connecting spaces within the body and can result in abnormal fluid flows and pain. In particular, the use of a flowable composite composition loaded optionally with antibiotic(s) is expected to provide markedly improved outcomes for the treatment of fistulas involving the GI tract or the reproductive tract. These include, for example, anal fistulas, vaginal fistulas, urinary tract fistulas, tracheoesophageal fistula, enteroenteric fistulas, enterocutaneous or colocutaneous fistulas. FIG. 9B is an illustration of a method of treating a fistula by delivering a flowable composite composition, generally loaded with antibiotic, into the fistula, in some embodiments after an initial cleaning procedure.

The method of fistula treatment is shown for illustration purposes for use in treating an anorectal fistula. In method 940, fistula 941 has primary opening 944 from rectum 950 and secondary opening 952 into buttock 954. Flexible trocar 960 has been inserted into fistula 941 through secondary opening 952. Flowable composite composition 962 has been delivered into fistula 941. Flexible trocar 960 can be pulled back through secondary opening 952 as flowable composite composition 962 fills fistula 941. In one embodiment, the surgeon would first surgically clean the fistula, including removal of infected tissue, prior to performing method 940. Performing method 940 would be expected to stop any bleeding associated with the cleaning procedure, while also filling and sealing the fistula. In some embodiments, the surgeon would use a composition that also has a visualization agent and monitor the delivery, such as with the use of ultrasound or x-ray imaging. Such monitoring may be used, for example, to ensure the fistula is sufficiently filled and/or may help to identify the path(s) of the fistula and suggest further treatment plans. For appropriate embodiments, the antibiotic-loaded flowable composite composition is expected to improve interventional outcomes via multiple pathways: rapid control of bleeding (if present), prevention of further ingress of contamination, prevention of infection, reduction of recurrences, reduction of need for further surgical intervention, and promotion of healing. In some embodiments, the surgeon would be able to perform a less invasive surgical procedure to prepare the fistula prior to the delivery of the antibiotic-loaded flowable composition into the fistula, which can further provide for improved surgical outcomes. In some cases, the fistulas are expected to resolve via this treatment.

Therapeutic agents loaded in the flowable composite compositions may include antibiotics approved for oral, topical, and/or prophylactic use for the treatment of fistulas, where effectivity of the agent is improved due to its local target proximity. For example, the following antimicrobials can be suitable: penicillins, cephalosporins, Vancomycin, Metronidazole, ciprofloxacin, Ampicillin, sulbactam, Anasyn, Ticarcillin, clavulanate potassium, Timentin, Clindamycin, doxycycline, or azithromycin, or a combination thereof. Broad spectrum antibiotics, which target both aerobic and anaerobic gram-negative organisms can be particularly suitable. In some embodiments, bactericidal agents, including peroxides (such as benzoyl peroxide) may be suitable to incorporate into the flowable composite compositions. In further embodiments, a therapeutic agent may include various classes of drugs. Drugs include, for instance, a pain reliever (such as Bupivacaine), a steroid, an anti-inflammatory drug (such as Diclofenac), a non-steroidal anti-inflammatory drug (NSAID), an anti-cancer drug, an anti-proliferative, nitrates (such as organic nitrates nitroglycerin rectal), an immunomodulator, a calcium channel blocker (such as Nifedipine, Diltiazem), a neuromuscular blocker (including botulotoxins), a vasodilator, an anxiolytic (including benzodiazepines, such as Diazepam), or combinations thereof. Examples of NSAIDS are Ibuprofen, Meclofenamate sodium, mefanamic acid, salsalate, sulindac, tolmetin sodium, ketoprofen, diflunisal, piroxicam, naproxen, etodolac, flurbiprofen, fenoprofen calcium, Indomethacin, celoxib, ketrolac, and nepafenac. The drugs themselves may be small molecules, proteins, RNA fragments, proteins, glycosaminoglycans, carbohydrates, nucleic acid, inorganic and organic biologically active compounds where specific biologically active agents include but are not limited to: enzymes, antibiotics, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations. In some embodiments, incorporation of a therapeutic agent(s) into the flowable composite composition enables continuous drug release directly to the fistula which can provide more convenient, comfortable, and effective local drug delivery as compared to manual insertion of creams to the primary opening of the fistula, such as intra-anally, or topically to the external region of the secondary opening of the fistula.

The flowable composite composition may have a concentration of the therapeutic agent that is similar to dosages suitable for topical, intramuscular, and/or intra-cavity use. In some embodiments, the concentration of the therapeutic agent is from about 1 mg/ml to about 200 mg/ml, in other embodiments from about 2 mg/ml to about 80 mg/ml, in other embodiments from about 2 mg/ml to about 40 mg/ml, in other embodiments from about 2 mg/ml to about 25 mg/ml, and in other embodiments from about 2.5 mg/ml to about 10 mg/ml. These modalities can be used effectively for controlled local delivery of a therapeutic dose over a selected period of time. In some embodiments, the flowable composite composition forms a solid depot that releases the therapeutic agent(s) in an effective dose for a period of about 2 days to about 3 weeks, in other embodiments for about 5 days to about 2 weeks, and in other embodiments for about 7 days to about 10 days. A person of ordinary skill in the art will recognize that additional ranges of concentrations and release times within the specific ranges above are contemplated and are within the present disclosure.

### EXAMPLES

### Example 1: Preparation of Flowable Sealant Formulations

This example describes the preparation of flowable sealant formulations in which a flowable composite composition is used as a sealant. An additional formulation, having no gelatin, is also prepared for use in a comparative study.

Flowable sealant formulations were prepared from a first hydrogel precursor, a second hydrogel precursor, unbuffered water-for-injection, and foamed gelatin. In particular, the first hydrogel precursor and the second hydrogel precursor, both as dry powders, were combined to form a precursor blend. The first hydrogel precursor was an eight-armed polyethylene glycol-based precursor having a 15,000 Da molecular weight and succinimidyl glutarate (SG) functional end groups (8A15k PEG SG, Jenkem Technologies USA (Jenkemusa)). The second hydrogel precursor was an eight-armed polyethylene glycol-based precursor having a 20,000 Da molecular weight and HCl-salted amine functional end groups (8A20k PEG amine-HCl, Jenkemusa). The first and second hydrogel precursor were combined in a 3:4 weight ratio in order to provide a precursor blend having an approximately equivalent number of SG and amine functional groups for crosslinking. For each formulation, unbuffered water-for-injection (WFI, pH 5.5) and a trace amount of FD&C Blue #1 (less than about 0.011% w/w) was added to a vial containing an amount of the precursor blend to form a precursor solution having a selected precursor concentration. The precursor solution was mixed and heated using a vortex mixer. For each precursor solution, the precursor blend dissolved rapidly in the WFI, as determined by the absence of particulates upon visual inspection. Each precursor solution was then transferred into a first 5 ml syringe and the syringe was capped. Next, a gelatin dispersion was prepared either by aspirating a desired amount of WFI into a second 5 mL syringe and inserting a selected amount of gelatin, in the form of fragments of foamed gelatin, into the syringe or by inserting a selected amount of gelatin, in the form of fragments of foamed gelatin, into the first syringe. The gelatin substrate fragments were prepared by cutting a gelatin substrate of a selected size into fragments. The amount of gelatin used in each of the formulations is shown in Table 1 as the size (area) of an approximately 1 mm thick sheet of foamed gelatin (Rousselot Quali-Pure®). The gelatin was allowed to soak in the WFI for about 2 to 4 minutes. Thereafter, the air was removed from the first syringe and the second syringe with the gelatin dispersion, if used, and a syringe luer was used to connect the first syringe and a second 5 ml syringe or the second syringe with the gelatin dispersion. The precursor solution and the gelatin dispersion, if used, were mixed by cycling the contents of the syringes between the first and the second syringe. One mixing cycle was the transfer of the contents of the syringes from a starting (original) syringe to the other syringe and back to the original syringe. Approximately 80 mixing cycles, a back and forth per cycle, were used to prepare the mixed solution. The mixed solution was then transferred to one of the syringes and the syringe luer was removed. Then, a 21G needle was attached to the syringe containing the mixed solution. The mixed solution was pushed through the needle and collected in a different syringe. This step was used to break up the gelatin particles. The preparation of Formulation C was thus completed with this step. Each of the remining mixed solutions was further processed by connecting a 23G needle to the different syringe and pushing the mixed solution through the needle. This additional step was used to further break up the gelatin particles and form Formulations A-B and D described in Table 1. A comparative formulation (Formulation CE), having no gelatin, was prepared following the above general method for the preparation of Formulations A-B and D. The concentration of hydrogel precursors listed in Table 1 is relative to the total solution. The relative viscosity of each formulation was qualitatively assessed by comparison of relative flow down a smooth sheet of Delrin plastic.

TABLE 1

| | Concentration of | Amount of | Qualitative |
|---|---|---|---|
| | hydrogel | Gelatin, | assessment of |
| Formulation | precursors, wt % | cm² | relative viscosity |
| A | 20 | 32 | high |
| B | 20 | 16 | medium |
| C | 20 | 16 | medium |
| D | 10 | 16 | low |
| CE | 40 | — | medium-low |

### Example 2: Liver Defect Study (With Comparative Example)

This example evaluated test compositions prepared according to Example 1 for testing hemostasis in a porcine liver defect model. Comparisons were made to a comparative composition prepared without gelatin.

Part A. Test and Comparative Compositions. Flowable sealant test compositions and a comparative composition, prepared according to the formulations described in Example 1, were used in this study. Each of the compositions was blue in color due to the presence of the blue dye.

Part B. Laparoscopic Animal Defect Model Preparation and Laparoscopic Evaluation Procedures and Results. A single acute (Yorkshire) pig was used in this study. The liver was accessed laparoscopically. A series of defects were created in both the left and right medial lobes of the liver, according to Table 2. For Example L1, the liver was held laterally with graspers to provide tension while a puncture defect of approximately 3 mm in length and approximately 1 mm in depth was made using a stainless steel trocar. For Example L2, the liver was held laterally with graspers to provide tension while a capsular tear defect of approximately 1 cm in length and approximately 1 mm in depth was created on the surface of the liver using the stainless steel trocar. For Examples L3 and L4, the liver was held laterally with graspers while laparoscopic scissors were used to create an approximately 1.5 cm long cut (incision) into the edge of the liver. For Example L5, 2-3 mm long defects were made with a bovie scratch pad followed by scratching the surface with the stainless-steel trocar. For Example L6, a series of approximately 2 cm long defects were made by scratching the surface with the stainless-steel trocar. For Example L7, a puncture defect was made using the stainless steel trocar to penetrate the liver to a depth of approximately 4 mm. Defect severity was evaluated using a SBSS Spot grade bleed score, as shown in Table 3.

TABLE 2

| | | Bleed | Test | Pressure | Time to |
|---|---|---|---|---|---|
| Example | Type of Defect | Score | Composition | Applied? | Hemostasis |
| L1 | Puncture | 1 | A | yes | 30 sec ¹ |
| L2 | Capsular Tear | 1 | A | yes | 60 sec |
| | (surface of liver) | | | | |
| L3 | Incision | 3 | A | yes | ¹ |
| | (edge of liver) | | | | |
| L4 | Incision | 3 | A | yes | 60 sec |
| | (edge of liver) ² | | | | |
| L5 | Raw | 2 | B | yes | 60 sec |
| | Surface/Ooze | | | | |
| L6 | Raw | 1 | B³ | yes | 60 sec |
| | Surface/Ooze | | | | |
| L7 | Puncture | 2 | D | yes | 60 sec ¹ |

¹ slight oozing remained
² same defect as L3
³ test composition was not subjected to a 23G needle step

TABLE 3

| Spot | | | | | | |
|---|---|---|---|---|---|---|
| Grade^{TM} | 0 | 1 | 2 | 3 | 4 | 5 |
| Verbal Descriptor | None | Minimal | Mild | Moderate | Severe; | Extreme; |
| | | | | | not immediately life | immediately life |
| | | | | | threatening | threatening |
| Visual Descriptor | Dry | Oozing | Pooling | Flowing | Streaming | Gushing |

Prior to the creation of each of the selected defects, a delivery formulation was chosen with input from the surgeon, and the test composition was prepared accordingly. A clean gauze was wetted using clean, sterile saline and introduced into the laparoscopic site with laparoscopic graspers. Then, immediately following the creation of each of the selected defects, the test composition was delivered laparoscopically to the defect site via a stainless-steel trocar having a 1.6 mm internal diameter.

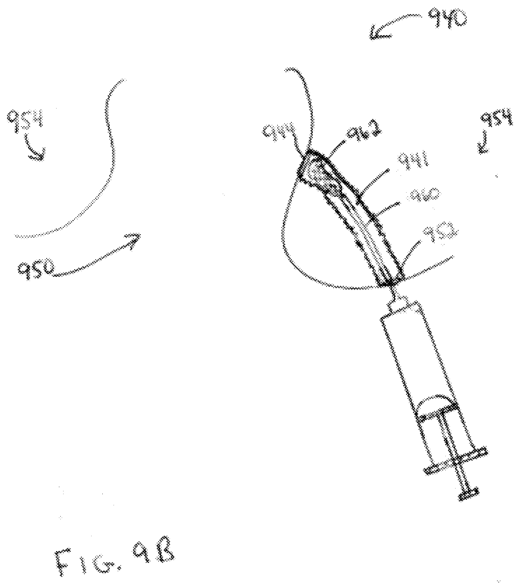

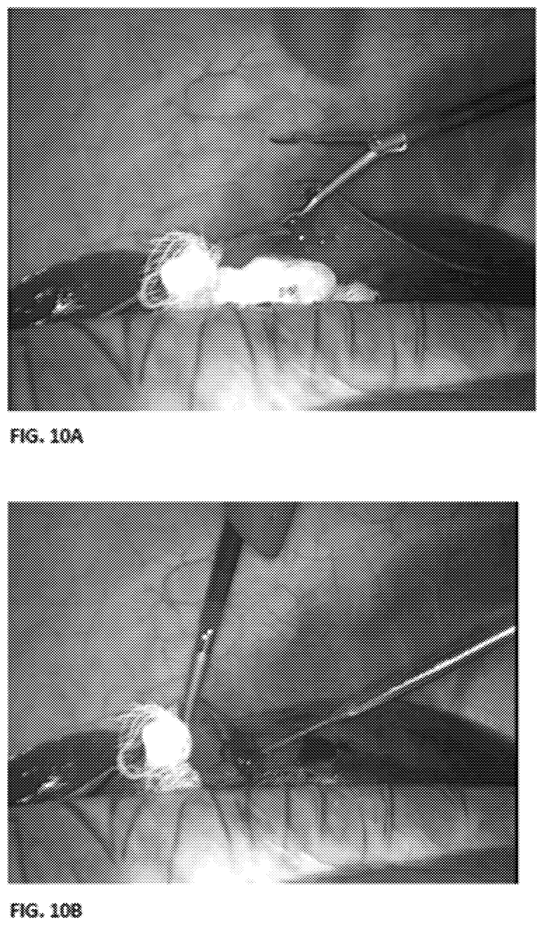

In Example L1, the test composition selected to treat the puncture defect was the high viscosity formulation A. At 30 seconds post-delivery, pressure was applied to the delivered composition with the wet gauze. Excess of the delivered composition was wiped off with gauze and/or washed away with saline irrigation while the gelled portion of the delivered composition remained and effectively provided wound stabilization. After about 30 seconds, only slight oozing remained. In Example L2, the test composition selected to treat the capsular tear on the surface of the liver was also the high viscosity formulation A. At 60 seconds post-delivery, pressure was applied to the delivered composition with the wet gauze. Excess of the delivered composition was wiped off with dry gauze. Hemostasis was achieved at 60 second post-delivery. In Example L3, the test composition selected to treat the incision on the edge of the liver was again the high viscosity formulation A. One flap of the defect created by the incision was lifted with the graspers and the test composition was delivered along the (bleeding) side of the tissue flap. The tissue flap was then pushed back into place and minimal pressure was applied with the wet gauze at approximately 30 seconds post-delivery. Hemostasis was not fully achieved with an initial application. FIGS. 10A-C are photographs taken during this procedure. FIG. 10A shows the edge incision defect. FIG. 10B shows the trocar being used to deliver the composition into the defect.

FIG. 10C shows that the tissue flaps have been sealed together and hemostasis has been partially achieved via the delivered composition. In Example L4, the treated yet oozing defect from Example L3 was treated again with the high viscosity formulation A. In this example, 2 ml of the test composition was delivered to the defect without lifting the tissue flap and remained in place. Gentle pressure was applied after delivery by gently pinching the defect between the wet gauze with the use of the grasper. Gentle pressure was also applied inward toward the defect site, using the wet gauze. Hemostasis was achieved at 60 seconds post-delivery. Note that the test composition for Example L4 was prepared without the 23 G needle step. The surgeon was blinded to the change in the test composition preparation and noted a difference in the injection force needed for delivery.

In Example L5, the test composition selected to treat raw surface/ooze was the medium viscosity formulation B. Two separate deliveries were made to the defect site, with pressure applied with gauze at 30 seconds after each delivery. After the first delivery, the delivered composition flowed off the site with saline irrigation. Hemostasis was achieved after the second delivery, which is attributed to not enough of the formulation being applied during the first delivery. In Example L6, the test composition selected to treat the raw surface/ooze defect was again the medium viscosity formulation B, except the composition was prepared without the 23G needle step. The surgeon was blinded to the change in the test composition preparation and noted a difference in the injection force needed for delivery, which is believed to be related to a larger particle size of gelatin in the test composition prepared without the 23G needle step. Hemostasis was achieved at 60 seconds post-delivery.

In Example L7, the test composition selected to treat the puncture defect was the low viscosity formulation D. At 30 seconds post-delivery, pressure was applied to the delivered composition with the wet gauze. After about 30 seconds, only slight oozing remained.

The results of the laparoscopic testing show that all of the test compositions were able to be effectively delivered laparoscopically to provide hemostasis or near hemostasis in about one minute or less. The high viscosity test composition was shown to be effective for treating a puncture defect, a capsular tear, and a relatively long and challenging edge incision with a bleed score of 3. The medium viscosity test composition was shown to be effective for treating oozing surface wounds. A low viscosity test composition was also shown to be effective in treating a puncture wound. This study shows that a single formulation can be used for multiple types of defects and/or formulations can be designed for a particular type of defect.

Part C. Open Animal Defect Model Preparation and Open Surgical Procedures and Results. Following the completion of the laparoscopic procedure from Part B, the pig was opened along the anterior (ventral) midline and the liver isolated. A series of defects were created in both the left and right medial lobes of the liver, and treated with test compositions according to Table 4. The defects were spatially separated from one another and from the previous defects so as to provide independent results. For Examples S1 through S4, abrasion defects were made using a bovie scratch patch. Each of the abrasion defects covered a surface of approximately 2 cm×2 cm. Defects for Examples S3 and S4 were made on a section of the liver that was at an approximate 45° angle with the horizontal plane, while S1 and S2 were relatively horizontal. For Example S5, a crater defect was made using an 8 mm biopsy punch to penetrate the liver to a depth of approximately 4 mm. For Example S6, an incision of approximately 2 cm in length and approximately 0.5 cm in depth was created on the surface of the liver using a scalpel. Defect severity was evaluated using a SBSS Spot grade bleed score, as shown in Table 3.

TABLE 4

| | | | Test | Delivery | Pressure | |
|---|---|---|---|---|---|---|
| Example | Type of Defect | Defect Severity | Composition | Tool | Applied? | Time to Hemostasis |
| S1 | Abrasion | 1 | B | syringe | yes¹ | 2 min |
| S2 | Abrasion | 1 | D | syringe | yes¹ | 2 min |
| S3 | Abrasion | 1 | D | syringe | no | Not |
| | (45° angle) | | | | | achieved |
| S4 | Abrasion | 1 | CE | syringe | no | 3 min ² |
| | (45° angle) | | | | | |
| S5 | Crater | 2 | C | syringe | yes | 2 min |
| S6 | Incision | 2 | B | syringe | yes | 4 min |

¹ after irrigation
² slight oozing remained

Prior to the creation of each of the selected defects, a selected delivery formulation was prepared with input from the surgeon. A clean gauze was wetted using clean, sterile saline. Then, immediately following the creation of each of the selected defects, 2 ml of the selected test composition was delivered to the defect site through the open surgical site directly from the barrel of one of the 5 ml syringes used to prepare the test composition.

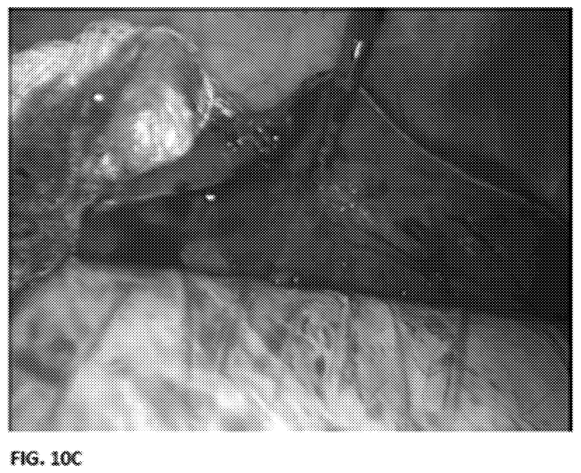

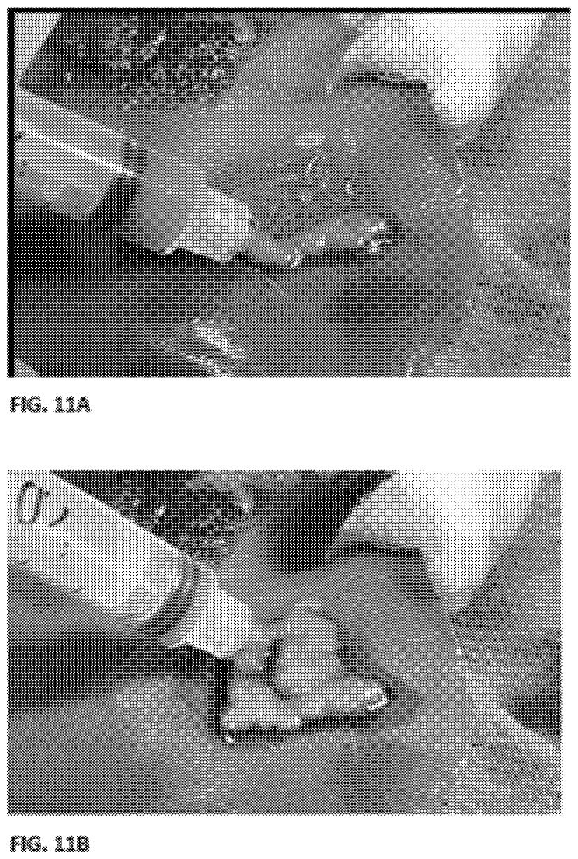

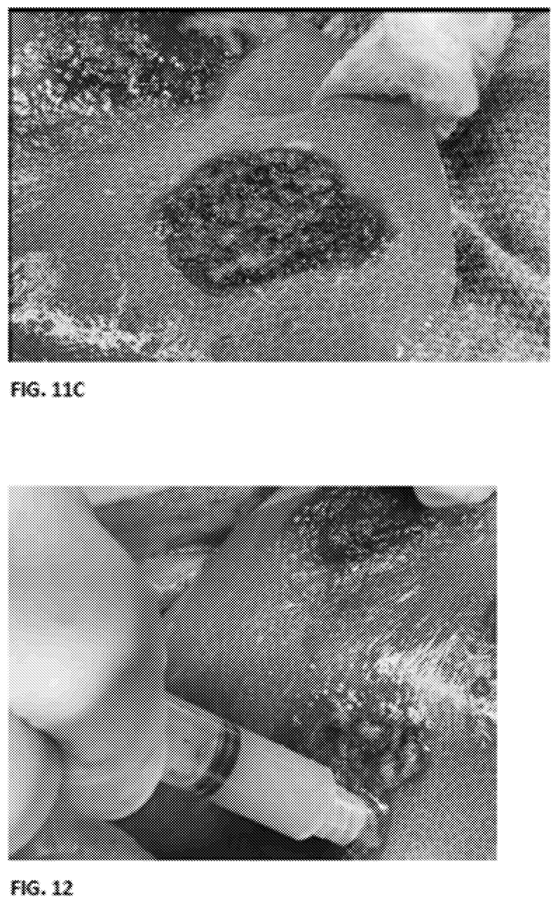

In Example S1, the test composition selected to treat the abrasion defect was the medium viscosity formulation B. Excess of the delivered composition was washed away with saline irrigation and then pressure was applied with the wet gauze. At 2 minutes the defect site was checked, and it was noted that hemostasis had been achieved. FIGS. 11A and 11B are photographs of the delivery of the composition onto this defect. FIG. 11C shows that hemostasis has been achieved via the delivered composition. In Example S2, the test composition selected to treat the abrasion defect was the low viscosity formulation D. Excess of the delivered composition was washed away with saline irrigation and then pressure was applied with the wet gauze. Hemostasis was also achieved at approximately 2 minutes post-delivery. In Example S3, the test composition selected to treat the abrasion defect was again the low viscosity formulation D. In this 45° placement experiment, it was found that the majority of the test composition slid off of the defect site. Prolonged pressure (approximately 3 minutes) was applied yet hemostasis was not achieved. The lack of hemostasis for Example S3 in comparison to Example S4, discussed below, seems to be correlated with the test composition D having a lower viscosity and a higher rate of sliding off of the defect site than test composition CE. FIG. 12 is a photograph of the delivery of the composition onto this 45° angle surface.

In Example S4, medium-low viscosity comparative composition CE (having 40 wt % of hydrogel precursors but no gelatin) was selected to treat the abrasion defect. In this comparative 45° placement experiment, it was found that the majority of the test composition slid off of the defect site before forming a gel. Prolonged pressure was applied to the defect site. Slight oozing remained at approximately 3 minutes post-delivery.

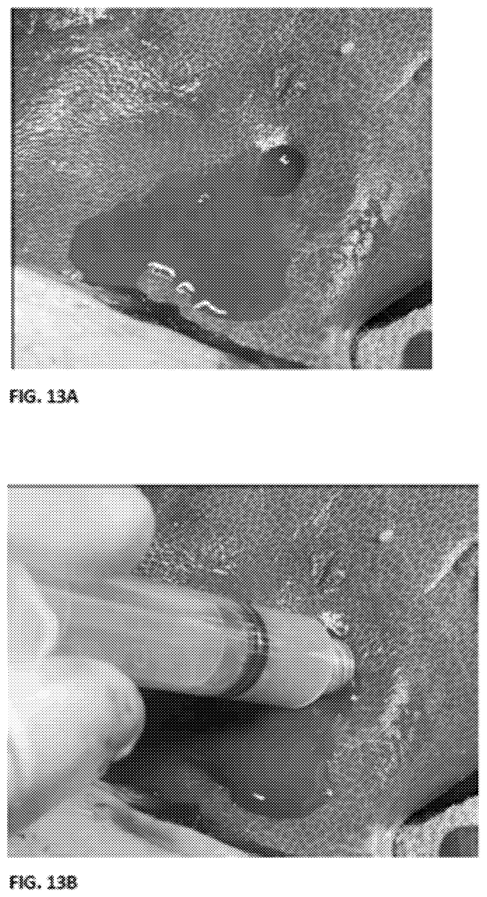

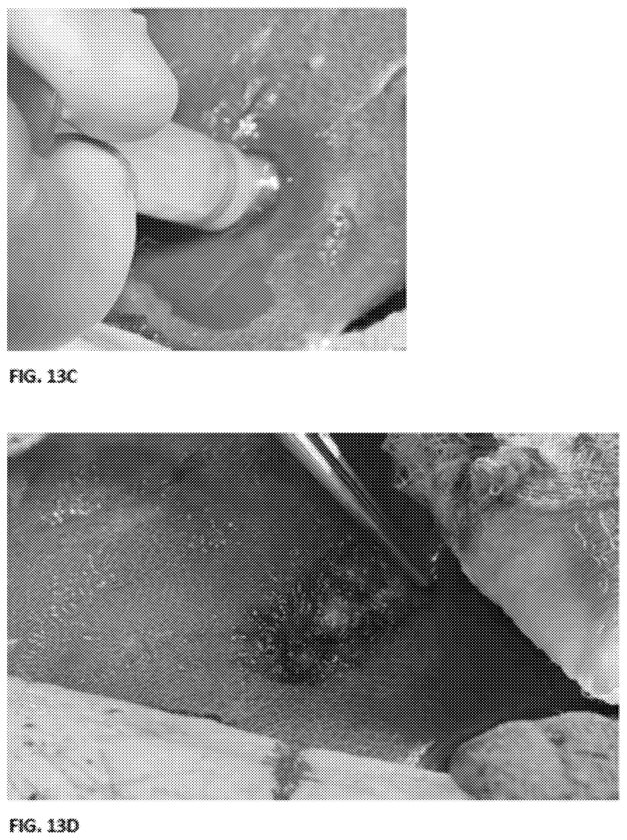

In Example S5, medium viscosity formulation C was used as the test composition for treating the crater defect. Formulation C was similar to formulation B yet used crosslinked gelatin instead of non-crosslinked gelatin. The test composition was applied by inserting the open end of the syringe into the bottom of the defect and injecting the composition as the syringe was slowly withdrawn from the defect. After delivery, pressure was applied with the wet gauze. Hemostasis was achieved at approximately 2 minutes post-delivery. FIGS. 13A-D are photographs taken during this procedure. FIG. 13A shows the crater defect. FIG. 13B shows the tip of the syringe being introduced into the defect. FIG. 13C shows the syringe barrel having been pushed further into the defect. FIG. 13D shows that hemostasis has been achieved via the delivered composition.

In Example S6, the test composition selected to treat the incision defect was the medium viscosity formulation B. The test composition was applied inside of the wound and on top of the wound. Pressure was applied with the wet gauze for 2 minutes after the delivery. The removal of the gauze caused some re-bleeding. Additional pressure was applied in addition to lateral compression of the wound via the wet gauze for a period of 2 minutes. Hemostasis was achieved at approximately 4 minutes post-delivery.

The results of the open surgical testing show that medium and low viscosity formulations can be used to treat minimal to mild bleeding defects. The results also demonstrate that angled defects sites pose sealing challenges, although higher viscosity products may be more effective. The results suggest that a combination of a relatively high weight percent of precursors and the use of granular gelatin may provide for a composition for angled sites that forms a gel fast enough to balance the flow characteristics of the gelatin-based formulations. Furthermore, as shown in Example 5, entrapping of air in the formulations can also be used to modify the flowability of gelatin-based formulations and thereby improve performance at angled sites. The results also show that hemostasis was achieved for two challenging defects (a crater and an incision) with the use of two different medium viscosity compositions. The test composition used in Example S5 was able to provide hemostasis of the crater defect in 2 minutes. The test composition used in Example S6 was able to provide hemostasis of the incision in 4 minutes.

This study demonstrated that a composition with hydrogel precursors and gelatin can be useful for treating various types of bleeding defects using laparoscopic or open surgical procedures. Formulations with a higher viscosity may be useful for achieving hemostasis more quickly. Increased viscosity can be achieved by increasing the weight percent of the hydrogel precursors and/or the amount of the hydrophilic polymer used. Granular gelatin in a formulation can function as a bulking agent, which can be advantageous in treating certain types of bleeding defects. It is anticipated that crosslinked or non-crosslinked gelatin as well as air entrapment can be used and enable tailoring of the results. This study shows that formulations can designed for a particular type of defect and conveniently delivered directly from the barrel of a single syringe or via a cannula.

### Example 3: Abdominal Tissue Flap Study

This example demonstrates the performance of a flowable composite composition prepared according to Example 1 for sealing potential spaces associated with dissected tissue flaps.

Following the completion of the open surgical procedure from Part C of Example 2, an abdominal tissue pocket defect was created in the pig model during an open surgical procedure by dissecting between the abdominal muscle flap and the subcutaneous tissues. The tissue pocket defect in this study simulates, for example, the environment of tissues after an abdominoplasty procedure.

A flowable composite composition was prepared according to formulation A from Example 1. The overlying tissue flap of the defect was lifted with graspers, and then the sealant composition was delivered onto the muscle flap from the superior region to the inferior region of the defect using a wave pattern. Approximately 2 ml of the sealant composition was delivered into the tissue pocket defect through the open surgical site directly from the barrel of one of the 5 ml syringes used to prepare the sealant composition. The overlying tissue flap was then positioned over the muscle flap and pressure was applied to the overlying tissue flap for 1 minute. To test the sealing of the tissue flap, an attempt was made to locate the original dissection plane. The fact that this was difficult was an indication of the rapid sealing of the tissue flap. Extensive re-dissection was undertaken to attempt to open and re-establish the original tissue planes of the defect, which indicates the effectiveness of the sealing of the tissue flap defect. This study shows that the flowable composite composition can be used to effectively eliminate potential spaces that could form pockets for seroma or hematoma formation.

### Example 4: Simulated In Situ Performance Study

This example evaluates the performance of a series of flowable composite compositions in a simulated in situ environment.

Simulated in situ flowable composite compositions were prepared from a first hydrogel precursor, a second hydrogel precursor, a sheet of foamed gelatin, unbuffered water-for-injection, a trace amount of FD&C Blue #1 (less than about 0.011% w/w), and buffering salts. For these studies, the first hydrogel precursor was an eight-armed polyethylene glycol-based precursor having a 15,000 Da molecular weight and succinimidyl succinate (SS) functional end groups (8A15k PEG SS, Jenkem Technologies USA (Jenkemusa)). The second hydrogel precursor was a four-armed polyethylene glycol-based precursor having a 20,000 Da molecular weight and amine functional end groups (4A20k PEG amine, Jenkemusa). The first and second hydrogel precursor were provided as powders. Each composition included the first and the second hydrogel precursor in a 3:8 weight ratio in order to provide a precursor blend having an approximately equivalent number of SS and amine functional groups for crosslinking. The buffer compounds were added to the compositions to simulate contact with physiological fluids present in an in situ environment, which are naturally buffered.

In a first step, the gelatin was prepared by inserting a selected amount of gelatin, in the form of fragments of a foamed gelatin sheet, into the barrel of a 10 ml syringe (hereinafter “the gelatin syringe”). To this gelatin syringe was added a selected amount of a 0.2 M sodium phosphate dibasic buffer solution. Air was removed from the gelatin syringe, and the mixture was held within the gelatin syringe at room temperature for about 10 minutes to allow the gelatin to soak. Hydration and properties of the gelatin were not expected to be altered by the buffer in the soak.

Next, an amine stock solution was prepared by adding a selected mass of the second hydrogel precursor to a vial and then adding a selected amount of the dibasic buffer solution to achieve the desired concentration. After the gelatin soaking period, the gelatin syringe was connected to an empty 10 ml syringe (hereinafter “Syringe 2”) via a syringe luer. The contents of the gelatin syringe were cycled between the gelatin syringe and Syringe 2. One mixing cycle was the transfer of the contents of the syringes from a starting (original) syringe to the other syringe and back to the original syringe. Approximately 80 mixing cycles, a back and forth per cycle, were used to prepare the initial gelatin suspension. The initial gelatin suspension was then transferred to one of the syringes and the syringe luer was removed. Then, the gelatin syringe and Syringe 2 were connected via an 18G (gauge) micro-emulsifier, and the mixture was exchanged through the micro-emulsifier approximately 80 times. This step was used to further break up the gelatin particles. The gelatin suspension was then transferred to one of the syringes and the micro-emulsifier was removed. Then, a selected amount of the amine stock solution was added to the syringe containing the gelatin suspension (hereinafter the “Gelatin/Amine Syringe”).

In a next step, an ester stock solution was prepared by adding a selected mass of the first hydrogel precursor to a vial and then adding a selected amount of a 0.01 M sodium phosphate monobasic buffer solution to achieve the desired concentration. Then, a selected amount of the ester stock solution was added to an empty 10 ml syringe (hereinafter “the Ester Syringe”).

Next, the Gelatin/Amine Syringe was connected to the Ester Syringe via a syringe luer connector, and the contents of the syringes were cycled between the two syringes for 5 seconds to prepare a simulated in situ flowable composite composition. Each prepared composition was deposited onto a template to form one or more depot samples.

The prepared compositions were evaluated based on four performance criteria. The first criteria is that a passing composition forms a fully gelled, solid depot. Second, a passing precursor composition flows out of syringe kit with reasonable force. The syringe kit includes a syringe and a luer connector or a micro-emulsifier. Third, the prepared composition forms a depot(s) with a uniform visual appearance. Uniform visual appearance is assessed by the uniformity of the color of the depots. Fourth, the prepared composition forms a depot(s) that remain intact after 24 hours at 37° C. in a 1×PBS solution (approximately 137 mM NaCl, 2.7 mM KCl, 10 mM Na₂HPO₄, and 1.8 mM KH₂PO₄). Table 5 shows how a series of prepared compositions and corresponding precursors performed with respect to these criteria, where the compositions differed by composition. Note that all of the samples passed the second criteria with respect to injectability.

TABLE 5

| | Concentration | | | |
|---|---|---|---|---|
| | of hydrogel | Concentration | | |
| Compo- | precursors, | of gelatin, | Amount of | All Performance |
| sition | wt % | wt % | Solvent, mg | Criteria Met? |
| 001 | 2.5 | 3.8 | 5000.0 | Yes |
| 002 | 2.5 | 3.8 | 5000.0 | Yes |
| 004 | 0.5 | 3.9 | 5000.0 | No ^{1, 4} |
| 005 | 1.0 | 4.0 | 5000.0 | No ^{1, 4} |
| 006 | 2.5 | 8.1 | 5000.0 | Yes |
| 007 | 2.5 | 13.8 | 5000.0 | No ^{3, 4} |
| 008 | 2.5 | 10.6 | 5000.0 | Yes |
| 009 | 1.5 | 3.8 | 5000.0 | No 4 |
| 011 | 2.5 | 3.7 | 5000.0 | Yes |
| 012 | 5.0 | 3.7 | 5000.0 | Yes |
| 013 | 2.5 | 13.1 | 5000.0 | No ^{3, 4} |
| 017A | 5.0 | 16.0 | 1000.0 | No ^{3, 4} |
| 017B | 5.0 | 1.9 | 10,000.0 | Yes |
| 019 | 5.0 | 17.5 | 5000.0 | No ^{3, 4} |

¹ gel was not fully formed; center of depot was not solid
² not able to flow out of syringe assembly with reasonable force
³ lacked visual uniformity
⁴ failed to remain intact after 24 hours at 37° C. in 1x PBS

Compositions 001-005, 009, and 011-012 have a relatively constant concentration of gelatin and solvent, but a concentration of hydrogel precursors that varies from 0.5 wt % to 5 wt %. The performance is seen to vary among these compositions. This data suggests that the performance of the compositions, especially with respect to criteria 1 and 4, is improved by using a higher concentration of the hydrogel precursors. Each of these compositions which had a precursor concentration above about 2.5 wt % met all of the performance criteria.

Compositions 006-008 and 013 were prepared with the same concentration of hydrogel precursors (2.5 wt %), but with a different concentration of gelatin (ranging from 8.1 wt % to 13.8 wt %). The performance is seen to vary among these compositions. Compositions 006 and 008, having a concentration of gelatin of 8.1 wt % and 10.6 wt %, respectively, met all of the performance criteria. This data suggests that the performance of the compositions, especially with respect to criteria 3 and 4, is improved by using a concentration of gelatin that is no more than about 11-13 wt %.

This simulated in situ study to indicates that the performance of the flowable composite compositions in situ can be adjusted by varying the concentration of the hydrogel precursors and/or the concentration of the hydrophilic particulate material. Higher concentrations of hydrogel precursors and moderate concentrations of the hydrophilic particulate material were found to be associated with better performance.

### Example 5: Flowability Study

This example evaluated the effects of entrapped air on the flow performance of a set of model formulations.

Model formulations were prepared using the general procedures described in Example 1, with the exception that the model formulations were not prepared without hydrogel precursors and were not pushed through a needle after the mixing cycles.

In particular, model formulations were prepared from unbuffered water-for-injection (WFI, pH 5.5) and foamed gelatin, with the relative amount of added entrapped air being an additional variable. The model formulations are summarized in Table 6. For each formulation, WFI, trace amount of FD&D Blue #1 (less than about 0.011% w/w), and a selected amount of gelatin, in the form of fragments of foamed gelatin, was added to a first 10 ml syringe. The gelatin was allowed to soak in the WFI for about 2 to 4 minutes. Thereafter, the air was removed from the first syringe, and the first syringe and a second 10 mL syringe were connected using a syringe luer. The gelatin solution was mixed by cycling the solution between the first and the second syringe. One set of formulations (G09-G15) was mixed gently for several cycles to incorporate as little additional air as possible. These formulations are indicated as having no air added, so only the air introduced naturally by the ingredients. The “no air added” formulations ranged from 5.0 wt % to 2.5 wt % of gelatin. The remaining formulations (G16-G19) were mixed with a number of mixing cycles to incorporate (entrap) a selected amount of additional air. The amount of added air in the formulations as prepared ranged from 34% to 72%. The amount of added air in the formulations was calculated as the percent difference between the volume of the formulation after the mixing cycles and the initial volume of the formulation in the first syringe relative to the total volume with the added air, which was adjusted to add a selected volume of air that was subsequently incorporated into the combined composition. Graduation marks on the syringes were used to measure volumes.

TABLE 6

| | Concentration | | Amount of | | | |
|---|---|---|---|---|---|---|
| | of Gelatin, | Air Added | Added | Flow Factor | Flow Factor | Excess Liquid |
| Formulation | wt % | (Y/N) | Air, vol % | (%)_{avg} | (% )_{StdDev} | (Y/N) |
| G09 | 5.0 | N | 0 | 0% | 0% | N |
| G10 | 4.0 | N | 0 | 0% | 0% | Y |
| G11 | 3.5 | N | 0 | 46% | 11% | Y |
| G12 | 4.0 | N | 0 | 60% | 1% | Y |
| G13 | 3.5 | N | 0 | 63% | 1% | Y |
| G14 | 3.0 | N | 0 | 79% | 21% | Y |
| G15 | 2.5 | N | 0 | 100% | 0% | Y |
| G16 | 3.5 | Y | 72 | 48% | 3% | N |
| G17 | 3.5 | Y | 37 | 90% | 9% | N |
| G18 | 4.0 | Y | 71 | 65% | 7% | N |
| G19 | 4.0 | Y | 34 | 71% | 6% | N |

Each prepared formulation was deposited onto a Delrin® substrate which had been marked with a “start line” and an “end line,” 200 cm from the start line. Each formulation was deposited onto a start line with the substrate oriented 0 degrees on the lab bench. The substrate was then angled to 90 degrees on the bench and a timer was started. An image was taken at the 60 second mark. The distance that each deposited formulation travelled during the 60 second was measured. This distance in centimeters was divided by 200 cm to determine the “flow factor.” If the deposited formulation did not flow past the start line, it was measured as 0 cm travelled, therefore a flow factor of 0%. Formulations G9 and G10, having 5.0 wt % and 4.0 wt % gelatin, respectively and no added air, each had a flow factor of 0%. As noted in Table 6, “no air added” formulations G10-G15 had some excess liquid dripping out of the bulk gelatin. This excess liquid was not used for measuring the flow factor of the formulation.

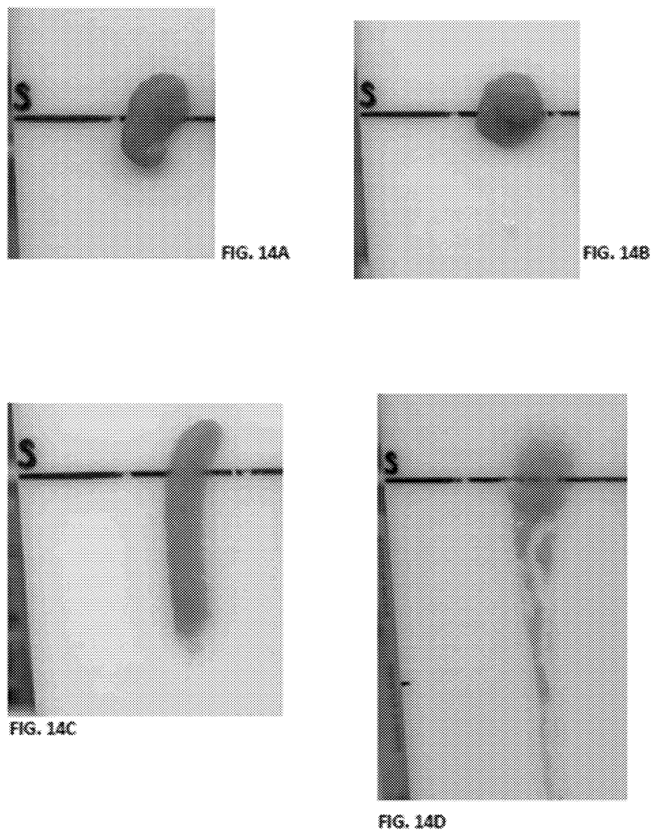

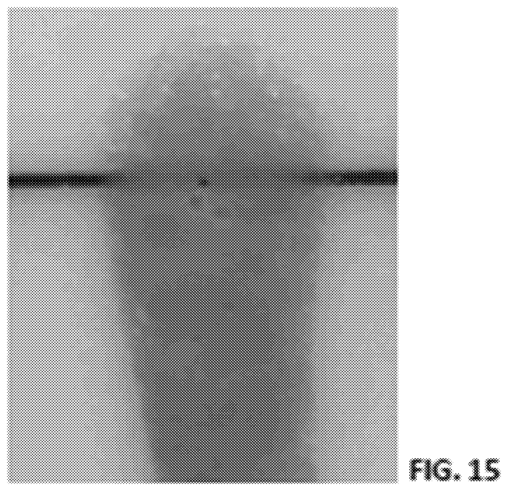

FIG. 14A shows a photograph of formulation G9 at the end of the 60 second flowability test. This formulation showed no flow. FIG. 14B shows a photograph of formulation G10 at the end of the 60 second flowability test. This formulation showed only excess liquid flow. FIG. 14C shows a photograph of formulation G11 at the end of the 60 second flowability test. This formulation showed gelatin flow plus excess liquid flow. FIG. 14D shows a photograph of formulation G15 at the end of the 60 second flowability test. This formulation showed 200 cm of gelatin flow. All formulations without entrapped air either fully flowed down or had excess fluid flow.

Formulations G16-G19 were prepared with from 72, 37, 71, and 34 volume percent of added air, respectively. These formulations showed no signs of excess fluid flow. Furthermore, the amount of added air was shown to impact the flow factor of the formulations as well as the appearance of the formulations. For formulation G16, having 3.5 wt % gelatin and 72 vol % added air, the flow factor was 48%. FIG. 15 shows a photograph of formulation G16 at the end of the 60 second flowability test. Air bubbles are visible. For formulation G17, having 3.5 wt % gelatin and 37 vol % added air, the flow factor was 90%. Similarly, the flow factor for G18, having 4.0 wt % gelatin and 71 vol % added air, was lower than the flow factor for G19, having 4.0 wt % gelatin and less added air.

The results of this study show that the flowability of a gelatin formulation is affected by both the percent gelatin and amount of entrapped air, which suggests that the flowability may be tuned by adjusting these parameters. This study also suggests that incorporating additional air could provide improvements to flowable sealant formulations having hydrogel precursors, such as described in Example 1, with respect to maintaining the composition at the point of delivery. These results demonstrate that foaming can be an effective approach for maintaining the composition at the point of delivery for applications where the location provides challenges for flowing of the composition off of the wound or defect.

### FURTHER INVENTIVE CONCEPTS

X1. A foamed flowable composite composition comprising a blend of:
1) a first component comprising a) a granular hydrophilic material, b) hydrogel precursors comprising an electrophilic-hydrogel precursor having a plurality of electrophilic functional groups and a nucleophilic-hydrogel precursor having a plurality of protonated amine groups, or c) a mixture of granular hydrophilic material and hydrogel precursors;
2) an aqueous solvent; and
3) and entrapped gas,
   wherein the hydrogel precursors are dissolved and the granular hydrophilic material is at least partially hydrated, with the proviso that if the first component comprises hydrogel precursors, the aqueous solvent has a pH of no more than about 6.5.
   X2. The foamed flowable composite composition of inventive concept X1 wherein the hydrogel precursors form a hydrogel in no more than about 60 seconds after contact with bodily fluids.
   X3. The foamed flowable composite composition of inventive concept X1 further comprising a visualization agent, a thickener, and/or a therapeutic agent.
   X4. The foamed flowable composite composition of inventive concept X3 wherein the visualization agent comprises an optical visualization agent, an x-ray visualization agent, an ultrasound imaging agent, or a combination thereof.
   X5. The foamed flowable composite composition of inventive concept X3 wherein the visualization agent comprises a dye.
   X6. The foamed flowable composite composition of inventive concept X3 wherein the visualization agent comprises microbubbles.
   X7. The foamed flowable composite composition of inventive concept X3 wherein the therapeutic agent comprises an anesthetic, an antibiotic, a steroid, a non-steroidal anti-inflammatory drug (NSAID), a chemotherapeutic, or a combination thereof.
   X8. The foamed flowable composite composition of inventive concept X3 wherein the therapeutic agent comprises an antibiotic.
   X9. The foamed flowable composite composition of inventive concept X1 wherein the entrapped gas comprises air, carbon dioxide, nitrogen, or a combination thereof.
   X10. The foamed flowable composite composition of inventive concept X1 wherein the entrapped gas is air.
   X11. The foamed flowable composite composition of inventive concept X1 wherein the foamed flowable composite composition is flowable from a syringe with an 18 gauge needle.
   X12. The foamed flowable composite composition of inventive concept X1 wherein the foamed flowable composite composition is flowable from a cannula.
   X13. The foamed flowable composite composition of inventive concept X1 wherein the aqueous solvent has a pH of no more than about 6.5.
   X14. The foamed flowable composite composition of inventive concept X1 wherein the first component comprises, separately, the granular hydrophilic material, the first hydrogel precursor, and the second hydrogel precursor.
   X15. The foamed flowable composite composition of inventive concept X1 wherein the first component comprises the granular hydrophilic material and a separate dry mixture of the first hydrogel precursor and the second hydrogel precursor.
   X16. The foamed flowable composite composition of inventive concept X1 wherein the first component comprises a dry mixture of the granular hydrophilic material, the first hydrogel precursor, and the second hydrogel precursor.
   A1. A flowable composite composition comprising a mixture of a granular hydrophilic material, hydrogel precursors comprising an electrophilic-hydrogel precursor having a plurality of electrophilic functional groups and a nucleophilic-hydrogel precursor having a plurality of protonated amine groups, and an aqueous solvent at a pH of no more than about 6.5, wherein the hydrogel precursors are dissolved and the granular hydrophilic material is at least partially hydrated.
   A2. The flowable composite composition of inventive concept A1 wherein the aqueous solvent comprises water for injection, unbuffered saline, an alcohol, dimethylcarbonate, or a combination thereof.
   A3. The flowable composite composition of inventive concept A1 wherein the aqueous solvent is unbuffered saline.
   A4. The flowable composite composition of any one of inventive concepts A1-A3 wherein the granular hydrophilic material comprises gelatin, collagen, hyaluronic acid, polyethylene glycol-based hydrogels or other natural or synthetic hydrogels.
   A5. The flowable composite composition of any one of inventive concepts A1-A4 wherein the granular hydrophilic material is biodegradable and comprises crosslinked gelatin.
   A6. The flowable composite composition of any one of inventive concepts A1-A5 wherein the granular hydrophilic material comprises gelatin and the hydrogel precursors comprise a PEG-electrophilic hydrogel precursor having a plurality of arms having terminal reactive electrophilic groups and a PEG-nucleophilic hydrogel precursor having a plurality of arms having terminal protonated amine groups.
   A7. The flowable composite composition of any one of inventive concepts A1-A6 wherein the electrophilic functional groups and the protonated amine groups are in a molar ratio range of about 1:0.5 to 1:1.5.
   A8. The flowable composite composition of any one of inventive concepts A1-A6 wherein the electrophilic functional groups and the protonated amine groups are in a molar ratio range of about 1:0.75 to 1:0.95 or about 1:1.05 to 1:1.25 or about 1:0.95 to 1:1.05.
   A9. The flowable composite composition of any one of inventive concepts A1-A8 wherein the flowable composite composition comprises a suspension.
   A10. The flowable composite composition of any one of inventive concepts A1-A9 wherein the flowable composite composition has from about 2.5 wt % to about 25 wt % solids.
   A11. The flowable composite composition of any one of inventive concepts A1-A10 comprising from about 5 wt % to about 50 wt % of the hydrogel precursors.
   A12. The flowable composite composition of any one of inventive concepts A1-A10 comprising from about 10 wt % to about 40 wt % of the hydrogel precursors.
   A13. The flowable composite composition of any one of inventive concepts A1-A12 wherein the hydrogel precursors form a hydrogel in no more than about 3 minutes after contact with bodily fluids.
   A14. The flowable composite composition of any one of inventive concepts A1-A13 wherein the hydrogel precursors form a hydrogel in no more than about 60 seconds after contact with bodily fluids.
   A15. The flowable composite composition of any one of inventive concepts A1-A14 wherein the hydrogel degrades in no more than about 14 days by bodily processes in vivo.
   A16. The flowable composite composition of any one of inventive concepts A1-A15 wherein the flowable composite composition crosslinks to form a hydrogel composite that is non-swelling.
   A17. The flowable composite composition of any one of inventive concepts A1-A16 wherein the composition has a storage stability against restriction of flowability of at least about 10 minutes at room temperature.
   A18. The flowable composite composition of any one of inventive concepts A1-A17 further comprising an additive comprising a visualization agent, a thickener, a therapeutic agent or a combination thereof.
   A19. The flowable composite composition of inventive concept A18 wherein the flowable composite composition comprises the additive in an amount up to about 5 wt %.
   A20. The flowable composite composition of inventive concept A18 or A19 wherein the visualization agent comprises an optical visualization agent, an x-ray visualization agent, an ultrasound imaging agent, or a combination thereof.
   A21. The flowable composite composition of inventive concept A18 or A19 wherein the visualization agent comprises a dye.
   A22. The flowable composite composition of inventive concept A18 or A19 wherein the visualization agent comprises microbubbles.
   A23. The flowable composite composition of inventive concept A18 or A19 wherein the therapeutic agent comprises an anesthetic, an antibiotic, a steroid, a non-steroidal anti-inflammatory drug (NSAID), a chemotherapeutic, or a combination thereof.
   A24. The flowable composite composition of inventive concept A18 or A19 wherein the therapeutic agent comprises an antibiotic.
   A25. The flowable composite composition of any one of inventive concepts A1-A24 wherein the composition is foamed.
   A26. The flowable composite composition of inventive concept A25 wherein the density is from about 35% to about 95% of a corresponding un-foamed composition.
   A27. The flowable composite composition of inventive concept A25 wherein the entrapped gas in the foam is air.
   A28. The flowable composite composition of any one of inventive concepts A1-A27 wherein the flowable composite composition is flowable from a syringe with an 18 gauge needle.
   A29. The flowable composite composition of any one of inventive concepts A1-A28 wherein the aqueous solvent has a pH of no more than about 6.
   A30. The flowable composite composition of any one of inventive concepts A1-A29 wherein the aqueous solvent has no added buffer.
   A31. The flowable composite composition of any one of inventive concepts A1-30 wherein the flowable composite composition has pH of no more than about 6.
   A32. The flowable composite composition of any one of inventive concepts A1-A31 wherein the electrophilic-hydrogel precursor comprises a multiarm polyethylene glycol with succinimidyl functional groups and the nucleophilic-hydrogel precursor comprises a multiarm polyethylene glycol with protonated amine functional groups.
   A33. The flowable composite composition of any one of inventive concepts A1-A32 wherein the electrophilic-hydrogel precursor and the nucleophilic-hydrogel precursor independently have a molecular weight of about 2K Da to about 50K Da and from 4 to 8 arms.
   A34. The flowable composite composition of any one of inventive concepts A1-A33 wherein the electrophilic-hydrogel precursor comprises a reactive ester.
   A35. The flowable composite composition of any one of inventive concepts A1-A34 wherein the electrophilic-hydrogel precursor has a first hydrophilic polymer core and/or the nucleophilic-hydrogel precursor has second hydrophilic polymer core comprising polyethylene glycol, polyvinyl alcohol, polylactic acid, polyoxazoline, copolymers thereof, or mixtures thereof.
   B1. A method for forming a flowable composite composition useful as a tissue sealant, the method comprising:
dissolving an electrophilic-hydrogel precursor in an aqueous solvent to form an electrophile hydrogel precursor solution;
dissolving a nucleophilic-hydrogel precursor in an aqueous solvent to form a nucleophile hydrogel precursor solution; and
blending insoluble hydrophilic granular material into a hydrogel precursor solution; and
combining the electrophilic precursor solution and the nucleophilic precursor solution if separately formed, prior to or following blending the insoluble hydrophilic granular material to form the flowable composite composition comprising a mixture of the electrophilic hydrogel precursor solution, the nucleophile hydrogel precursor solution and the insoluble hydrophilic granular material.
   B2. The method of inventive concept B1 wherein the insoluble hydrophilic granular material is provided as friable chunks, and wherein the method further comprises contacting the friable chunks with aqueous solvent and pulverizing the chunks in the solvent to form the granular material prior to the mixing.
   B3. The method of inventive concept B1 comprising blending the granular hydrophilic material with aqueous solvent to form an at least partially hydrated granular material prior to blending with the electrophilic hydrogel precursor solution and the nucleophile hydrogel precursor solution.
   B4. The method of inventive concept B3 wherein the at least partially hydrated granular material is first blended with the electrophile hydrogel precursor solution and then blended with the nucleophile hydrogel precursor solution or wherein the at least partially hydrated granular material is first blended with the nucleophile hydrogel precursor solution and then blended with the electrophile hydrogel precursor solution.
   B5. The method of inventive concept B3 wherein the electrophile hydrogel precursor solution is first blended with the nucleophile hydrogel precursor solution to for a hydrogel precursor solution and then blending the hydrogel precursor solution with the at least partially hydrated granular hydrophilic material.
   B6. The method of inventive concept B3 wherein dry powders of the electrophile hydrogel precursor and the nucleophile hydrogel precursor are combined together with aqueous solvent to simultaneously for a hydrogel precursor solution comprising the electrophile precursor solution and the nucleophile precursor solution.
   B7. The method of inventive concept B3 wherein the at least partially hydrated granular hydrophilic material is combined with the electrophilic hydrogel precursor solution and the nucleophile precursor solution, which then are blended together to form a flowable composite solution.
   B8. The method of inventive concept B1 wherein the blending further comprises entrapping biocompatible gas in the flowable composite composition to form a foam.
   B9. The method of inventive concept B8 wherein the blending involves pulverizing hydrophilic material to form the granules and simultaneously entrap air to form a foam.
   B10. The method of inventive concept B8 wherein the entrapping gas comprises whipping the flowable composite composition after the granular material is substantially hydrated.
   B11. The method of inventive concept B8 wherein a specific volume of air is blended into the flowable composite composition.
   B12. The method of inventive concept B11 wherein the added volume of air is from about 0.1 cc to about 2 cc.
   B13. The method of inventive concept B11 wherein resulting density of the flowable composite composition is from about 35% to about 95% of the density of the flowable composite composition without entrapped air, as evaluated by the liquid volume and weight of the components.
   B14. The method of any one of inventive concepts B1-B13 wherein the electrophilic-hydrogel precursor and the nucleophilic-hydrogel precursor are provided as powders.
   B15. The method of inventive concept B8 wherein the flowable composite composition is foamed with an ultrasound contrast agent.
   B16. The method of inventive concept B8 wherein the biocompatible gas comprises CO₂, air, or nitrogen incorporated by bubbling during blending.
   B17. The method of inventive concept B15 wherein the ultrasound contrast agent comprises gas filled microbubbles.
   B18. The method of any one of inventive concepts B1-B17 further comprising incorporating a therapeutic agent into the flowable composite composition.
   B19. The method of inventive concept B18 wherein the therapeutic agent is added as an aqueous solution at a pH of no more than about 6.
   B20. The method of inventive concept B18 wherein the therapeutic agent is an antibiotic.
   B21. The method of any one of inventive concepts B1-B20 further comprising adding a visualization agent.
   B22. The method of inventive concept B21 wherein the visualization agent is a blue dye.
   B23. The method of any one of inventive concepts B1-B22 further comprising delivering the flowable composite composition through an atomizer.
   C1. A method for the delivery of a medical sealant, the method comprising:
placing a quantity of a flowable composite composition on or in or near a defect associated with tissue to facilitate healing wherein the flowable composite composition comprises a mixture of a granular hydrophilic material, an electrophilic-hydrogel precursor, a nucleophilic-hydrogel precursor, and an aqueous solvent at a pH of no more than about 6.5, wherein the hydrogel precursors are dissolved.
   C2. The method of inventive concept C1 wherein the defect is a suture line, a puncture wound, a bullet wound, a cavity, a perforation, a gouge, a biopsy punch hole, a graft interface, an abrasion wound, a tissue harvest site, or a combination thereof.
   C3. The method of inventive concept C1 wherein the defect is a fistula.
   C4. The method of inventive concept C3 wherein the flowable composite composition further comprises an antibiotic.
   C5. The method of inventive concept C1 wherein the flowable composite composition is a foam.
   C6. The method of inventive concept C5 wherein the foamed flowable composite composition has a density from about 35% to about 95% of the density of the flowable composite composition without entrapped air.
   C7. The method of inventive concept C2 wherein the defect has a depth of about 0.1 mm to about 5 cm and/or a surface area of from about 0.1 cm² to about 500 cm².
   C8. The method of inventive concept C1 wherein the defect is associated with a laparoscopic procedure.
   C9. The method of inventive concept C1 wherein the defect is caused by a medical procedure and wherein the placing is performed in conjunction with the medical procedure.
   C10. The method of any one of inventive concepts C1-C9 wherein the medical seal forms a fluid barrier.
   C11. The method of inventive concept C10 wherein the defect has a structure that forms a liquid or gas leak and wherein the fluid barrier comprises a hemostatic barrier, a liquid barrier, a gas barrier, or a combination thereof.
   C12. The method of any one of inventive concepts C1-C11 wherein the defect is a bleeding defect.
   C13. The method of inventive concept C1 wherein the defect is caused by a medical procedure and wherein placing the medical sealant fills gaps where blood or serum could accumulate.
   C14. The method of inventive concept C13 wherein the medical procedure involves surgery forming two adjacent tissue planes and wherein the placing comprises placement between the two tissue planes to adhere the adjacent tissue planes together.
   C15. The method of inventive concept C14 further comprising use of suture, staples, bandages or combinations thereof to secure the edges of the adhered tissue plane.
   C16. The method of inventive concept C1 further comprising applying compression to the flowable composition for no more than about 2 minutes.
   C17. The method of inventive concept C1 wherein the defect is a bleeding defect and wherein the placing results in hemostasis within about 3 minutes.
   C18. The method of inventive concept C1 wherein the defect is a bleeding defect and wherein the placing results in hemostasis within about 1 minute.
   C19. The method of inventive concept C1 wherein the placing comprises injection, spraying, or delivering with a cannula or dropper.
   C20. The method of inventive concept C1 further comprising making the flowable composite composition prior to the placing step, wherein the making step comprises mixing the granular hydrophilic material, the electrophilic-hydrogel precursor, the nucleophilic-hydrogel precursor, and the aqueous solvent in an appropriate sequence to form the flowable composite composition.
   C21. The method of inventive concept C20 wherein the electrophilic-hydrogel precursor and the nucleophilic-hydrogel precursor are provided as powders.
   C22. The method of inventive concept C1 wherein the flowable composite composition crosslinks to form a non-swellable composite material.
   C23. The method of inventive concept C1 wherein the granular hydrophilic material comprises gelatin, collagen, crosslinked hyaluronic acid, polyethylene glycol hydrogels, or other absorbable natural or synthetic hydrogel materials.
   C24. The method of inventive concept C1 wherein the flowable composite composition has from about 2.5 wt % to about 25 wt %. solids.
   C25. The method of inventive concept C1 wherein the defect is an incision separating two tissue layers, wherein the placing is between the two tissue layers to seal the tissues together.
   C26. The method of inventive concept C25 further comprising securing the edge of the joined tissue layers with suture, staples, bandages or a combination thereof.
   C27. The method of inventive concept C1 wherein the flowable composite composition further comprises a therapeutic agent.
   C28. The method of inventive concept C1 further comprising following achievement of hemostasis, removing any uncured flowable composite composition from the surface of a deposit.
   C29. The method of any one of inventive concepts C1-C28 wherein the placing step comprises injecting through an atomizer.
   C30. The method of any one of inventive concepts C1-C29 wherein the delivered volume of flowable composite composition is from about 1 mL to about 50 mL.
   D1. A flowable composite composition comprising a dispersion of a granular hydrophilic material in an aqueous solution comprising an aqueous solvent, a dissolved electrophilic-hydrogel precursor and a dissolved nucleophilic-hydrogel precursor, wherein the aqueous solvent is at a pH of no more than about 6.5 and wherein the granules are not fully hydrated.
   D2. The flowable composite composition of inventive concept D1 wherein the aqueous solvent is unbuffered saline or water for injection.
   D3. The flowable composite composition of inventive concepts D1 or D2 wherein the delivery site has oozing fluid.
   D4. The flowable composite composition of any one of inventive concepts D1-D3 wherein the granular hydrophilic material comprises gelatin, collagen, hyaluronic acid, polyethylene glycol-based hydrogels or other natural or synthetic hydrogels.
   D5. The flowable composite composition of any one of inventive concepts D1-D4 wherein the granular hydrophilic material is biodegradable and comprises crosslinked gelatin.
   D6. The flowable composite composition of any one of inventive concepts D1-D5 wherein the granular hydrophilic material comprises gelatin and the dissolved electrophilic-hydrogel precursors comprise PEG.
   D7. The flowable composite composition of any one of inventive concepts D1-D6 wherein the flowable composite composition comprises an additive in an amount up to about 5 wt %.
   D8. The flowable composite composition of inventive concept D7 wherein the additive comprises s visualization agent, a thickener, a therapeutic agent or a combination thereof.
   D9. The flowable composite composition of any one of inventive concepts D1-D8 wherein the hydrogel precursors form a hydrogel in no more than about 3 minutes after contact with bodily fluids.
   D10. The flowable composite composition of any one of inventive concepts D1-D9 wherein the hydrogel precursors form a hydrogel in no more than about 60 seconds after contact with bodily fluids.
   D11. The flowable composite composition of any one of inventive concepts D1-D10 wherein the hydrogel degrades in no more than about 14 days by bodily processes in vivo.
   D12. The flowable composite composition of any one of inventive concepts D1-D11 wherein the flowable composition is non-swelling.
   D13. The flowable composite composition of any one of inventive concepts D1-D12 wherein the composition has a storage stability against restriction of flowability of at least about 10 minutes at room temperature.
   D14. The flowable composite composition of any one of inventive concepts D1-D13 further comprising a visualization agent and/or a therapeutic agent.
   D15. The flowable composite composition of inventive concept D14 wherein the visualization agent comprises an optical visualization agent, an x-ray visualization agent, an ultrasound imaging agent, or a combination thereof.
   D16. The flowable composite composition of inventive concept D14 or D15 wherein the visualization agent comprises a dye.
   D17. The flowable composite composition of inventive concept D14 or D15 wherein the visualization agent comprises microbubbles.
   D18. The flowable composite composition of inventive concept D14 wherein the therapeutic agent comprises an anesthetic, an antibiotic, a steroid, a non-steroidal anti-inflammatory drug (NSAID), a chemotherapeutic, or a combination thereof.
   D19. The flowable composite composition of inventive concept D14 wherein the therapeutic agent comprises an antibiotic.
   D20. The flowable composite composition of any one of inventive concepts D1-D19 wherein the composition is foamed.
   D21. The flowable composite composition of inventive concept D20 wherein the density is from about 35% to about 95% of a corresponding un-foamed composition.
   D22. The flowable composite composition of inventive concept D20 or D21 wherein the entrapped gas in the foam is air.
   D23. The flowable composite composition of inventive concept D1 wherein the flowable composite composition is flowable from a syringe with an 18 gauge needle.
   D24. The flowable composite composition of any one of inventive concepts D1-D23 wherein the aqueous solvent has no added buffer.
   E1. A method of treating a fistula, the method comprising:
injecting a flowable composite composition into an opening into a fistula, wherein the flowable composite composition comprises a dispersion of a hydrophilic granular material in an aqueous solution comprising an aqueous solvent, a dissolved electrophilic-hydrogel precursor and a dissolved nucleophilic-hydrogel precursor, wherein the aqueous solvent is at a pH of no more than about 6.5.
   E2. The method of inventive concept E1 wherein the flowable composite composition comprises the composition of any one of claims 1-40 or inventive concepts A1-A35 or D1-D24.
   E3. The method of inventive concept E1 or E2 wherein the fistula is associated with the gastrointestinal tract, the urinary tract, or pulmonary location.
   E4. The method of inventive concept E1 or E2 wherein the fistula is an anal fistula.
   F1. A method for forming a foamed hydrogel composite, the method comprising:
directing a flowable composite composition through an atomizer, wherein the flowable composite composition comprises a dispersion of a hydrophilic granular material in an aqueous solution comprising an aqueous solvent, entrapped gas, a dissolved electrophilic-hydrogel precursor and a dissolved nucleophilic-hydrogel precursor.
   F2. The method of inventive concept F1 wherein the directing is performed by injection from a syringe.
   F3. The method of inventive concept F2 wherein the atomizer is attached with a luer connector to the syringe.
   F4. The method of inventive concept F2 wherein a catheter is attached to the syringe with a luer connector and the atomizer is attached to the catheter with a luer connector.
   F5. The method of inventive concept F1 wherein the flowable composite composition is a foam comprising entrapped air.
   F6. The method of inventive concept F5 wherein the foam of the flowable composite composition has a density from about 35% to about 95% of the density of the flowable composite composition without entrapped air.
   F7. The method of any one of inventive concepts F1-F6 wherein the delivered volume of flowable composite composition is from about 1 mL to about 50 mL.
   F8. The method of any one of inventive concepts F1-F7 wherein the flowable composite composition is directed on or in or near a defect associated with tissue to facilitate healing.
   F9. The method of inventive concept F8 wherein the defect is a suture line, a puncture wound, a bullet wound, a cavity, a perforation, a gouge, a biopsy punch hole, a graft interface, an abrasion wound, a tissue harvest site, a fistula, or a combination thereof.
   F10. The method of inventive concept F8 wherein the defect has a depth of about 0.1 mm to about 5 cm and/or a surface area of from about 0.1 cm² to about 500 cm².
   F11. The method of any one of inventive concepts F8-F10 wherein the defect is associated with a laparoscopic procedure.
   F12. The method of any one of inventive concepts F8-F11 wherein the defect is caused by a medical procedure and wherein the directing is performed in conjunction with the medical procedure.
   F13. The method of inventive concept F8 wherein the medical procedure involves surgery forming two adjacent tissue planes and wherein the placing comprises placement between the two tissue planes to adhere the adjacent tissue planes together.
   F14. The method of inventive concept F13 further comprising use of suture, staples, bandages or combinations thereof to secure the edges of the adhered tissue plane.
   F15. The method of any one of inventive concepts F1-F14 wherein following directing of the flowable composite composition results in contact with bodily fluids that induce crosslinking of the hydrogel precursors to form a hydrogel composite that forms a medical seal effective as a fluid barrier.
   F16. The method of inventive concept F15 wherein the fluid barrier comprises a hemostatic barrier, a liquid barrier, a gas barrier, or a combination thereof.
   F17. The method of any one of inventive concepts F1-F16 wherein the flowable composite composition comprises an aqueous solvent at a pH of no more than about 6.5, wherein the hydrogel precursors are dissolved.
   F18. The method of any one of inventive concepts F1-F17 wherein the flowable composite composition crosslinks to form a non-swellable composite material.
   F19. The method of any one of inventive concepts F1-F18 wherein the granular hydrophilic material comprises gelatin, collagen, crosslinked hyaluronic acid, polyethylene glycol hydrogels, or other absorbable natural or synthetic hydrogel materials.
   F20. The method of any one of inventive concepts F1-F19 wherein the flowable composite composition has from about 2.5 wt % to about 25 wt %. solids.
   F21. The method of any one of inventive concepts F1-F20 wherein the flowable composite composition comprises from about 2.5 wt % to about 25 wt % of the hydrogel precursors.
   F22. The method of any one of inventive concepts F1-F21 wherein the hydrogel precursors form a hydrogel in no more than about 3 minutes after contact with bodily fluids.
   F23. The method of any one of inventive concepts F1-F21 wherein the hydrogel precursors form a hydrogel in no more than about 60 seconds after contact with bodily fluids.
   F24. The method of any one of inventive concepts F1-F23 wherein the hydrogel degrades in no more than about 14 days by bodily processes in vivo.
   F25. The method of any one of inventive concepts F1-F24 wherein the flowable composite composition has a storage stability against restriction of flowability of at least about 10 minutes at room temperature.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art. A person of ordinary skill in the art is notified that the assertions above regarding the contemplation of subranges within explicit ranges are sincerely intended to provide explicit written description for the subranges, as clearly suggested, even though not explicitly written and that the subranges are not believed to change the character of the associated invention, although of course the specific values of parameters will certainly quantitively change corresponding results obtained, which could influence patentability even though the basic character of the invention may not be changing, in view of the potential nature of the state of the art known or unknown at filing given that inventiveness may follow from the factual details. A person of ordinary skill in the art is further notified that upper and lower values of explicit ranges and values within explicit ranges are intended to provide explicit written description for endpoints of subranges, furthermore explicit disclosure of upper and/or lower values of explicitly ranges of a certain feature are intended to be disclosed as upper and/or lower values for additional ranges, including subranges.

What is claimed is:

1. A foamed flowable composite composition comprising a blend of:
i) a first component comprising a) a granular hydrophilic material, b) hydrogel precursors comprising an electrophilic-hydrogel precursor having a plurality of electrophilic functional groups and a nucleophilic-hydrogel precursor having a plurality of protonated amine groups, or c) a mixture of granular hydrophilic material and hydrogel precursors;
ii) an aqueous solvent; and
iii) and entrapped gas,
wherein the hydrogel precursors are dissolved and the granular hydrophilic material is at least partially hydrated, with the proviso that if the first component comprises hydrogel precursors, the aqueous solvent has a pH of no more than about 6.5.

2. The foamed flowable composite composition of claim 1 wherein the aqueous solvent comprises water for injection, unbuffered saline, an alcohol, or a combination thereof.

3. The foamed flowable composite composition of claim 1 wherein the aqueous solvent is unbuffered saline.

4. The foamed flowable composite composition of claim 1 wherein the granular hydrophilic material comprises gelatin, collagen, hyaluronic acid, polyethylene glycol-based hydrogels or other natural or synthetic hydrogels.

5. The foamed flowable composite composition of claim 1 wherein the granular hydrophilic material is biodegradable and comprises crosslinked gelatin.

6. The foamed flowable composite composition of claim 1 wherein the granular hydrophilic material comprises gelatin and the hydrogel precursors comprise a PEG-electrophilic hydrogel precursor having a plurality of arms having terminal reactive electrophilic groups and a PEG-nucleophilic hydrogel precursor having a plurality of arms having terminal protonated amine groups.

7. The foamed flowable composite composition of claim 1 wherein the electrophilic functional groups and the protonated amine groups are in a molar ratio range of about 1:0.5 to 1:1.5.

8. The foamed flowable composite composition of claim 1 wherein the electrophilic functional groups and the protonated amine groups are in a molar ratio range of about 1:0.75 to 1:0.95 or about 1:1.05 to 1:1.25 or about 1:0.95 to 1:1.05.

9. The foamed flowable composite composition of claim 1 wherein the foamed flowable composite composition comprises a suspension.

10. The foamed flowable composite composition of claim 1 wherein the foamed flowable composite composition has from about 2.5 wt % to about 25 wt % solids.

11. The foamed flowable composite composition of claim 1 wherein the first component is a mixture that comprises from about 5 wt % to about 50 wt % of the hydrogel precursors.

12. The foamed flowable composite composition of claim 1 wherein the first component is a mixture that comprises from about 10 wt % to about 40 wt % of the hydrogel precursors.

13. The foamed flowable composite composition of claim 1 wherein the first component comprises an additive in an amount up to about 5 wt %.

14. The foamed flowable composite composition of claim 13 wherein the additive comprises a visualization agent, a thickener, a therapeutic agent or a combination thereof.

15. The foamed flowable composite composition of claim 1 wherein the hydrogel precursors form a hydrogel in no more than about 3 minutes after contact with bodily fluids.

16. The foamed flowable composite composition of claim 1 wherein the hydrogel degrades in vivo in no more than about 14 days by bodily processes in vivo.

17. The foamed flowable composite composition of claim 1 wherein the foamed flowable composition is non-swelling.

18. The foamed flowable composite composition of claim 1 wherein the foamed flowable composition has a storage stability against restriction of flowability of at least about 10 minutes at room temperature.

19. The foamed flowable composite composition of claim 1 wherein the density is from about 35% to about 95% of a corresponding un-foamed composition.

20. The foamed flowable composite composition of claim 1 wherein the aqueous solvent has no added buffer.

21. The foamed flowable composite composition of claim 1 wherein the foamed flowable composite composition has an acidic pH.

22. The foamed flowable composite composition of claim 1 wherein the mixture of granular hydrophilic material and hydrogel precursors comprises from about 50 wt % to about 95 wt % of the granular hydrophilic material and from about 5 wt % to about 50 wt % of the hydrogel precursors.

23. The foamed flowable composite composition of claim 1 wherein the mixture of granular hydrophilic material and hydrogel precursors comprises from about 65 wt % to about 95 wt % of the granular hydrophilic material and from about 5 wt % to about 35 wt % of the hydrogel precursors.

24. The foamed flowable composition of claim 1 having from about 10 vol % to about 85 vol % entrapped gas.

25. The foamed flowable composition claim 1 wherein the first compound comprises a multiarm polyethylene glycol with succinimidyl functional groups and the second compound comprises a multiarm polyethylene glycol with protonated amine functional groups.
